(19) United States
(12) Patent Application Publication
(10) Pub. No.: US 2025/0163117 A1
(43) Pub. Date: May 22, 2025

(54) NOVEL INTERLEUKIN-2 POLYPEPTIDES

(71) Applicant: CURE GENETICS, CO., LIMITED, Suzhou (CN)

(72) Inventors: Lei Zhao, Suzhou (CN); Yuan Liu, Suzhou (CN); Rong Wang, Suzhou (CN); Yongji Jiang, Suzhou (CN)

(21) Appl. No.: 18/726,832

(22) PCT Filed: Jan. 6, 2023
(86) PCT No.: PCT/CN2023/070898

§ 371 Date: Jul. 5, 2024

(30) Foreign Application Priority Data
Jan. 7, 2022 (WO) PCT/CN2022/070639
Jul. 7, 2022 (WO) PCT/CN2022/104322

(51) Int. Cl. C07K 14/55; A61K 38/00; A61P 35/00; C12N 15/70

(52) U.S. Cl. CPC C07K 14/55; A61P 35/00; C12N 15/70; A61K 38/00

(57) ABSTRACT

Disclosed herein are novel interleukin 2 (IL-2) polypeptides. The IL-2 polypeptides provided herein can be, for example, truncated, shuffled, and/or circularly permutated variants of wildtype human IL-2. The IL-2 polypeptides provided herein can have, for example, reduced or abolished binding to IL-2Rα. Nucleic acids that encode these IL-2 polypeptides, vectors having such nucleic acids, and cells having such nucleic acids or vectors are also provided.

### 1. RELATED APPLICATIONS

This application claims priority to PCT Patent Application No. PCT/CN2022/070639, filed Jan. 7, 2022, and to PCT Patent Application No. PCT/CN2022/104322, Jul. 7, 2022, both of which are incorporated herein by reference in their entireties.

### 2. REFERENCE TO SEQUENCE LISTING SUBMITTED ELECTRONICALLY

This application incorporates by reference a Sequence Listing with this application, which is entitled “102A005WO03.XML,” created on Jan. 2, 2023, and having a size of 61,416 bytes.

### 3. FIELD

The present invention relates to molecular biology, cell biology, immunology, and cancer biology.

### 4. BACKGROUND

Due to its ability to active and expand cancer-targeting lymphocytes in vivo, interleukin-2 (IL-2) immunotherapy has been an attractive option for cancer treatment.

Recombinant human IL-2 (Proleukin®, Aldesleukin) was approved for metastatic melanoma and metastatic renal cell carcinoma. However, the use of IL-2 in patients is severely limited by its toxic effects and low efficacy.

Therefore, there is an unmet need for IL-2 variants with reduced toxicity compared to wildtype IL-2. The novel truncated, shuffled, and circularly permutated IL-2 polypeptides provided herein address this need and provide related advantages.

### 5. SUMMARY

Provided herein are interleukin-2 (IL-2) polypeptide comprising, from N-terminus to C-terminus, (i) helix 1 (“H1”), helix 3 (“H3”), helix 2 (“H2”), and helix 4 (“H4”); or (ii) H2, H3, H1, and H4; or a circularly permutated variant thereof; wherein H1, H2, H3, and H4 each have the amino acid sequences of SEQ ID NOs:2, 5, 7, and 10, respectively, or a variant thereof having up to five amino acid mutations.

In some embodiments of the IL-2 polypeptides provided herein, the circularly permutated variant has a cutting point at the C-terminus of H1, H2, H3 or H4.

In some embodiments, (1) H1 has the amino acid sequence of SEQ ID NO:2; (2) H2 has the amino acid sequence of SEQ ID NO:5, 13, 14, or 15; (3) H3 has the amino acid sequence of SEQ ID NO:7; or (4) H4 has the amino acid sequence of SEQ ID NO:10, 16, 17, or 18; or any combination thereof.

In some embodiments, IL-2 polypeptides provided herein further comprise: (1) linker 1 (“L1”) located at the N-terminus of H1; (2) linker 2 (“L2”) located at the C-terminus of H1; (3) linker 3 (“L3”) located at the C-terminus of H2; (4) linker 4 (“L4”) located at the C-terminus of H3; or (5) linker 5 (“L5”) located at the C-terminus of H4; or any combination thereof; wherein L1, L2, L3, L4, and L5 each have the amino acid sequences of SEQ ID NOs:1, 3, 6, 8, and 11, respectively, or a variant thereof having up to three amino acid mutations.

In some embodiments, IL-2 polypeptides provided herein comprise (1) L1; (2) L2, except when H1 is located at the C-terminus of the IL-2 polypeptide; (3) L3, except when H2 is located at the C-terminus of the IL-2 polypeptide; (4) L4, except when H3 is located at the C-terminus of the IL-2 polypeptide; and (5) L5, except when H4 is located at the C-terminus of the IL-2 polypeptide.

In some embodiments, (1) L1 has the amino acid sequence of SEQ ID NO:1 or 12; (2) L2 has the amino acid sequence of SEQ ID NO:3; (3) L3 has the amino acid sequence of SEQ ID NO:6; (4) L4 has the amino acid sequence of SEQ ID NO: 8; (5) L5 has the amino acid sequence of SEQ ID NO:11; or any combination thereof.

In some embodiments, IL-2 polypeptides provided herein comprise from N-terminus to C-terminus, H1, H3, H2, and H4. In some embodiments, IL-2 polypeptides provided herein have the amino acid sequence of SEQ ID NO:20, 21, or 22.

In some embodiments, IL-2 polypeptides provided herein comprise from N-terminus to C-terminus, H3, H2, H4, and H1. In some embodiments, IL-2 polypeptides provided herein have the amino acid sequence of SEQ ID NO:23, 24, or 25.

In some embodiments, IL-2 polypeptides provided herein comprise from N-terminus to C-terminus, H2, H4, H1, and H3. In some embodiments, IL-2 polypeptides provided herein have the amino acid sequence of SEQ ID NO:26, 27, or 28.

In some embodiments, IL-2 polypeptides provided herein comprise from N-terminus to C-terminus, H4, H1, H3, and H2. In some embodiments, IL-2 polypeptides provided herein have the amino acid sequence of SEQ ID NO:29, 30, or 31.

In some embodiments, IL-2 polypeptides provided herein comprise from N-terminus to C-terminus, H1, H4, H2, and H3. In some embodiments, IL-2 polypeptides provided herein have the amino acid sequence of SEQ ID NO:32, 33, or 34.

In some embodiments, IL-2 polypeptides provided herein comprise from N-terminus to C-terminus, H4, H2, H3, and H1. In some embodiments, IL-2 polypeptides provided herein have the amino acid sequence of SEQ ID NO:35, 36, or 37.

In some embodiments, IL-2 polypeptides provided herein comprise from N-terminus to C-terminus, H2, H3, H1, and H4. In some embodiments, IL-2 polypeptides provided herein have the amino acid sequence of SEQ ID NO:38, 39, or 40.

In some embodiments, IL-2 polypeptides provided herein comprise from N-terminus to C-terminus, H3, H1, H4, and H2. In some embodiments, IL-2 polypeptides provided herein have the amino acid sequence of SEQ ID NO:41, 42, or 43.

In some embodiments, IL-2 polypeptides provided herein are conjugated to an Fc domain, a Human Serin Albumin (HSA), an anti-HSA binder, a polyethylene glycol (PEG), or a lipid moiety.

In some embodiments, IL-2 polypeptides provided herein are linked to a targeting moiety that binds to a tumor-associate antigen or an antigen in the extracellular matrix in a tumor, an immunotherapeutic agent, an immune checkpoint modulator, or a peptide-MHC complex.

In some embodiments, IL-2 polypeptides provided herein (1) have reduced or abolished binding affinity to IL-2Rα; (2) bind to IL-2Rβ/γ with an affinity equal to or greater than wildtype human IL-2's binding affinity for IL-2Rβ/γ; (3) activate IL-2Rβ/γ to an extent or amount equal to or great than wildtype human IL-2 activates IL-2Rβ/γ; or (4) have negligible immunogenicity in human; or any combination thereof.

In some embodiments, provided herein are nucleic acids having a polynucleotide sequence encoding an IL-2 polypeptide provided herein. In some embodiments, the nucleic acid is an mRNA.

In some embodiments, provided herein are vectors comprising a nucleic acid provided herein.

In some embodiments, provided herein are host cells having a nucleic acid provided herein or a vector provided herein.

In some embodiments, provided herein are pharmaceutical compositions comprising an IL-2 polypeptide provided herein and a pharmaceutically acceptable carrier. In some embodiments, provided herein are pharmaceutical compositions comprising a nucleic acid provided herein and a pharmaceutically acceptable carrier.

In some embodiments, provided herein are kits comprising an IL-2 polypeptide provided herein. In some embodiments, provided herein are kits comprising a nucleic acid provided herein.

In some embodiments, provided herein are in vitro or ex vivo methods of activating an immune effector cell, comprising contacting the immune effector cell with an IL-2 polypeptide provided herein under conditions to activate the immune effector cell. In some embodiments, the immune effector cell is a T cell, a NK cell, a NKT cell, or a myeloid cell. In some embodiments, the immune effector cell is a T cell selected from a CD4+ T cell, a CD8+ T cell, a Th cell, a Tc cell, and a CAR T cell.

In some embodiments, provided herein are methods of enhancing an immune response in a subject in need thereof, comprising administering a therapeutically effective amount of an IL-2 polypeptide, a nucleic acid, or a pharmaceutical composition provided herein to the subject. In some embodiments, provided herein are uses of an IL-2 polypeptide, a nucleic acid, or a pharmaceutical composition provided herein in enhancing an immune response in a subject in need thereof. In some embodiments, the subject has cancer.

In some embodiments, provided herein are methods of treating cancer in a subject in need thereof comprising administering a therapeutically effective amount of an IL-2 polypeptide, a nucleic acid, or a pharmaceutical composition provided to the subject. In some embodiments, provided herein are uses of an IL-2 polypeptide, a nucleic acid, or a pharmaceutical composition provided in treating cancer in a subject in need thereof.

In some embodiments of the methods or uses provided herein, the cancer is a hematological cancer or a solid tumor.

In some embodiments, the IL-2 polypeptide, nucleic acid, or pharmaceutical composition provided is administered intratumorally, intravenously, subcutaneously, intraosseously, orally, transdermally, or sublingually.

In some embodiments of the methods or uses provided herein, the subject exhibits reduced adverse events as compared to a subject treated with human wildtype IL-2.

In some embodiments of the methods or uses provided herein, the IL-2 polypeptide is administered in combination with a second therapeutic agent. In some embodiments, the second therapeutic agent is a chemotherapeutic agent, a hormonal agent, an antitumor agent, an immunostimulatory agent, an immunomodulator, an immunotherapeutic agent or any combination thereof.

### 6. BRIEF DESCRIPTION OF DRAWINGS

FIG. 1 provides an illustration of a wildtype IL-2 (left) and a truncated IL-2 (trIL-2, right) with abolished binding to IL-2Rα (CD25).

FIG. 2 provides a diagram showing shuffling of the four α-helices of trIL-2 to form different circularly permutated trIL-2 molecules.

FIG. 3 provides computational protein structure modelling of circularly permutated trIL-2 (CP-trIL-2). The right panel shows the CP-trIL-2 polypeptide with P65D, F117K, and F124K mutations.

FIG. 4 provides western blot results showing the expression profiles of different CP-trIL-2 polypeptides.

FIG. 5 provides thermo-stability profiles of indicated CP-trIL-2 groups.

FIGS. 6-9 provide western blot analyses of pSTAT5 showing the activation profiles of each CP-trIL-2 group in NK-92 and HH cell lines. As shown, FIG. 6 provides results of the following CP-trIL-2 polypeptides: 011, 012, 013, 021, 022, and 023; FIG. 7 provides results for 031, 032, 033, 041, 042, and 043; FIG. 8 provides results for 051, 052, 053, 061, 062, and 063; and FIG. 9 provides results for 071, 072, 073, 081, 082, and 083.

FIGS. 10A-10B provide western blot analyses of pSTAT5 showing the activation profiles of eight CP-trIL-2 variants in human PBMCs (FIG. 10A) and mouse splenocytes (FIG. 10B).

FIGS. 11A-11C provide flow cytometry results showing the phosphorylation of STAT5 in human PBMCs in response to different doses of 001, 012, and 072 treatment. FIG. 11A provides results of the regulatory T cells (Treg). FIG. 11B provides results of the CD8+ T cells. FIG. 11C provides results of the NK cells.

FIGS. 12A-12C provide flow cytometry results showing the phosphorylation of STAT5 in mouse splenocytes in response to different doses of 001, 012, and 072 treatment. FIG. 12A provides results of the regulatory T cells (Treg). FIG. 12B provides results of the CD8+ T cells.

FIG. 12C provides results of the NK cells.

FIGS. 13A-13C provide proliferation profiles of human PBMC cells treated with 001, 012, and 072, respectively. FIG. 13A shows the expansion curves of the PBMC cells separately treated with 001, 012, and 072 at different concentrations. FIG. 13B shows the time curves of the PBMC cells separately treated with 001, 012, and 072 at 50 nM. FIG. 13C shows the Treg populations in expanded cells separately treated with 001, 012, and 072.

FIG. 14A-14B provide in vivo toxicity profiles of 001, 012, and 072. FIG. 14A shows the body weight curves of the mice separately treated with 001, 012, and 072 at different concentrations. FIG. 14B shows the wet-pulmonary weight of the mice separately treated with 001, 012, and 072 at different concentrations.

FIGS. 15A-15E provide in vivo proliferation profiles of various immune cells in response to treatment by 001, 012, and 072 at different concentrations. FIG. 15A provides the percentages of Treg cells. FIG. 15B provides the percentages of CD8+ T cells. FIG. 15C provides the percentages of NK cells. FIG. 15D provides the ratio of CD8+ T cells to Treg cells. FIG. 15E provides the ratio of NK cells to Treg cells.

FIGS. 16A-16B provide in vivo anti-tumor efficacy of rhIL-2 and 012. FIG. 16A shows the change of tumor volumes (mm³) days after start of treatment. FIG. 16B provides the final tumor volumes (mm³) of each group on Day 14, and the tumor growth inhibition (TGI) rate.

FIG. 17 provides results from surface plasmon resonance (SPR) showing the in vitro binding affinities of 001 and 012 to IL-2Rα, IL-2Rβ, common γ receptor, IL-2Rβγ, and IL-2Rαβγ, respectively.

### 7. DETAILED DESCRIPTION

Provided herein are circularly permutated IL-2 polypeptides and methods of uses thereof. IL-2 is a pluripotent cytokine produced primarily by activated CD4+ T cells and plays a crucial role in orchestrating the proliferation, survival, and function of immune effector cells (Teff), regulatory T cells (Treg) and natural killer (NK) cells to maintain immune homeostasis (Bluestone, N. Engl. J. Med., (2011) 365:2129-31; Boyman et al., Nat. Rev. Immunol. (2012) 12:180-90; Liao et al., Curr. Opin. Immunol. (2011) 23:598-604).

Wildtype human IL-2 is a four α-helical bundle type I cytokine with 133 amino acids. It was first identified as a T cell growth factor (Morgan et al., Science 193: 1007 (1976); Smith, Science 240, 1169-76 (1988); Bazan, Science 257, 410-13 (1992)), and subsequently shown to have broad actions. IL-2 mediates its action by binding to IL-2 receptors (IL-2R), which consist of up to three individual subunits (α subunit (IL-2Rα, CD25), R subunit (IL-2Rβ, CD122), γ subunit (common gamma chain, γ_{c}, or CD132)), the different association of which can produce receptor forms that differ in their affinity to IL-2 (Wang et al., Science 2005, 310, 1159-63). IL-2 binds to a heterotrimeric complex of the associated IL-2Rα, β, and γ, expressed on Treg cells, with a high affinity (K_{d}=10⁻¹¹M); it binds to a heterodimeric complex of IL-2 receptor consisting of the β and γ subunits, expressed on memory T cells and NK cells, with an intermediate affinity (K_{d}=10⁻⁹M); and it binds to the monomeric IL-2Rα subunit with a low affinity (K_{d}=10⁻⁸M). The binding of IL-2 with IL-2Rα itself has no signal transducing activity in immune cells. However, severe toxicity (e.g., capillary leakage syndrome (CLS)) was reported to be mediated by the binding between IL-2 and IL-2Rα expressed on non-immune cells, such as endothelial cells (Boyman et al., Science 2006, 311, 1924-7; Krieg et al., PNAS 2010, 107, 11906-11).

As provided in detail below, the novel polypeptides provided herein are based on circular permutation of truncated wildtype IL-2 (trIL-2), which are hereinafter referred to as “CP trIL-2.” Compared to the wildtype IL-2 and other IL-2 mutants in the art, the CP-trIL-2 polypeptides provided herein have at least the following advantages: abolished binding to IL-2Rα, which results in significant reduction in toxicity; improve the stability and/or efficacy; and/or low immunogenicity.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention. All publications, patent applications, patents, and other references mentioned herein are incorporated by reference in their entirety.

### 7.1 Definitions

Unless otherwise defined, all technical and scientific terms used herein have the same meaning as commonly understood by one of ordinary skill in the art to which this invention belongs. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry and hybridization described herein are those well-known and commonly used in the art.

The term “a” or “an” entity refers to one or more of that entity; for example, “a polypeptide,” is understood to represent one or more polypeptides.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The terms “polypeptide,” “peptide,” “protein,” and their grammatical equivalents as used interchangeably herein refer to polymers of amino acids of any length linked to one another through an amide bond, which can be linear or branched. It can include unnatural or modified amino acids or be interrupted by non-amino acids. A polypeptide, peptide, or protein can also be modified with, for example, disulfide bond formation, glycosylation, lipidation, acetylation, phosphorylation, or any other manipulation or modification. A linear polypeptide has two ends, one with a free amino group, referred to as the N-terminus, and one with a free carboxyl group, referred to as the C-terminus. A polypeptide can have one or more motifs, such as alpha helixes, arranged from its N-terminus to C-terminus. As used herein and understood in the art, a polypeptide having more than one motifs (e.g., helix 1, helix 2, helix 3, and helix 4) from N-terminus to C-terminus means that the primary sequences of these motifs are arranged from N-terminus to C-terminus of the polypeptide. For example, a polypeptide having, from N-terminus to C-terminus, helix 1, helix 2, helix 3, and helix 4 meaning that these helixes are arranged in that specific order from N-terminus to C-terminus. The motifs can be linked by one or more linker sequences.

The terms “polynucleotide,” “nucleic acid,” and their grammatical equivalents as used interchangeably herein mean polymers of nucleotides of any length and include DNA molecule (e.g., a cDNA or genomic DNA) and RNA (e.g., mRNA). The nucleotides can be deoxyribonucleotides, ribonucleotides, modified nucleotides or bases, and/or their analogs, or any substrate that can be incorporated into a polymer by DNA or RNA polymerase. The DNA or RNA molecules can include portions that are not naturally occurring, such as modified bases, modified backbone, deoxyribonucleotides in an RNA, etc. The nucleic acid molecule can be single-stranded or double-stranded.

A polypeptide, protein, nucleic acid, polynucleotide, vector, cell, or composition which is “isolated” is a polypeptide, protein, nucleic acid, polynucleotide, vector, cell, or composition which is in a form not found in nature. Isolated polypeptides, proteins, polynucleotides, vectors, cells, or compositions include those that are substantially free from at least one other component with which they are associated or found together in nature. Isolated polypeptides, proteins, polynucleotides, vectors, cells, or compositions include those which have been purified to a degree that they are no longer in a form in which they are found in nature. In some embodiments, a polypeptide, protein, nucleic acid, polynucleotide, vector, cell, or composition which is isolated is substantially pure.

The term “variant” and its grammatical equivalents as used herein in relation to a protein or a polypeptide with particular structure and functional features (the “reference protein” or “reference polypeptide”) refers to a different protein or polypeptide having a different amino acid sequence compared to the reference protein or reference polypeptide but maintains basic structure/functional features of the reference protein or polypeptide. The changes to an amino acid sequence can also be, for example, amino acid additions, deletions, or substitutions. The changes to an amino acid sequence can be conservative amino acid substitutions. In some embodiments, variants of a reference protein or polypeptide can contain minor or inconsequential changes to the reference protein or polypeptide. For example, minor or inconsequential changes can be amino acid changes (including substitutions, deletions, and insertions) that have little or no impact on the structure or biological activity of the polypeptide. Minor or inconsequential changes can also include additions of non-functional peptide sequence such as tag sequences to the reference protein/polypeptide.

The terms “identical,” percent “identity,” and their grammatical equivalents as used herein in the context of two or more polynucleotides or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software that can be used to obtain alignments of amino acid or nucleotide sequences are well-known in the art. These include, but are not limited to, BLAST, ALIGN, Megalign, BestFit, GCG Wisconsin Package, and variants thereof. In some embodiments, two polynucleotides or polypeptides provided herein are substantially identical, meaning they have at least 70%, at least 75%, at least 80%, at least 85%, at least 90%, and in some embodiments at least 95%, at least 96%, at least 97%, at least 98%, or at least 99% nucleotide or amino acid residue identity, when compared and aligned for maximum correspondence, as measured using a sequence comparison algorithm or by visual inspection. In some embodiments, identity exists over a region of the amino acid sequences that is at least about 10 residues, at least about 20 residues, at least about 40-60 residues, at least about 60-80 residues in length or any integral value there between. In some embodiments, identity exists over a longer region than 60-80 residues, such as at least about 80-100 residues, and in some embodiments the sequences are substantially identical over the full length of the sequences being compared. In some embodiments, identity exists over a region of the nucleotide sequences that is at least about 10 bases, at least about 20 bases, at least about 40-60 bases, at least about 60-80 bases in length or any integral value there between. In some embodiments, identity exists over a longer region than 60-80 bases, such as at least about 80-1000 bases or more, and in some embodiments the sequences are substantially identical over the full length of the sequences being compared, such as a polynucleotide sequence encoding a protein of interest.

The term “binding” or “binding affinity” as used herein generally refers to the strength of the sum total of noncovalent interactions between a binding moiety (e.g., an IL-2 polypeptide) and a target molecule (e.g., CD25). The binding of a binding moiety and a target molecule is a reversible process, and the affinity of the binding is typically reported as an equilibrium dissociation constant (KD). KD is the ratio of a dissociation rate (koff or kd) to the association rate (kon or ka). The lower the KD of a binding pair, the higher the affinity. A variety of methods of measuring binding affinity are known in the art, such as isothermal titration calorimetry (ITC), surface plasmon resonance (SPR), MicroScale Thermophoresis (MST), and Bio-Layer Interferometry (BLI), any of which can be used for purposes of the present disclosure. For example, the affinity of an IL-2 polypeptide to various forms of the IL-2 receptor can be determined by SPR, using standard instrumentation such as a BIAcore instrument (GE HEALTHCARE) and receptor subunits. Alternatively, binding affinity of IL-2 polypeptides to different forms of the IL-2 receptor can be evaluated using cell lines known to express one or the other form of the receptor. In some embodiments, the “KD” or “KD value” can be measured by assays known in the art, for example by a binding assay. The KD can be measured in a radiolabeled antigen binding assay (RIA) (Chen, et al., (1999) J. Mol Biol 293:865-881), using BLI or SPR by Biacore.

The term “vector,” and its grammatical equivalents as used herein refer to a vehicle that is used to carry genetic material (e.g., a polynucleotide sequence), which can be introduced into a host cell, where it can be replicated and/or expressed. Vectors applicable for use include, for example, expression vectors, plasmids, phage vectors, viral vectors, episomes and artificial chromosomes, which can include selection sequences or markers operable for stable integration into a host cell's chromosome. Additionally, the vectors can include one or more selectable marker genes and appropriate expression control sequences. Selectable marker genes that can be included, for example, provide resistance to antibiotics or toxins, complement auxotrophic deficiencies, or supply critical nutrients not in the culture media. Expression control sequences can include constitutive and inducible promoters, transcription enhancers, transcription terminators, and the like which are well known in the art. When two or more polynucleotides are to be co-expressed, both polynucleotides can be inserted, for example, into a single expression vector or in separate expression vectors. For single vector expression, the encoding polynucleotides can be operationally linked to one common expression control sequence or linked to different expression control sequences, such as one inducible promoter and one constitutive promoter. The introduction of polynucleotides into a host cell can be confirmed using methods well known in the art. It is understood by those skilled in the art that the polynucleotides are expressed in a sufficient amount to produce a desired product (e.g., an IL-2 polypeptide provided herein), and it is further understood that expression levels can be optimized to obtain sufficient expression using methods well known in the art.

As used herein, the term “encode” and its grammatical equivalents refer to the inherent property of specific sequences of nucleotides in a polynucleotide or a nucleic acid, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein. Unless otherwise specified, a “nucleotide sequence encoding an amino acid sequence” or a “polynucleotide sequence encoding an amino acid sequence” are used interchangeably and include all nucleotide sequences that are degenerate versions of each other and that encode the same amino acid sequence. Nucleotide sequences that encode proteins and RNA can include introns.

The term “treat” and its grammatical equivalents as used herein in connection with a disease or a condition, or a subject having a disease or a condition refer to an action that suppresses, eliminates, reduces, and/or ameliorates a symptom, the severity of the symptom, and/or the frequency of the symptom associated with the disease or disorder being treated. For example, when used in reference to a cancer or tumor, the term “treat” and its grammatical equivalents refer to an action that reduces the severity of the cancer or tumor, or retards or slows the progression of the cancer or tumor, including (a) inhibiting the growth, or arresting development of the cancer or tumor, (b) causing regression of the cancer or tumor, or (c) delaying, ameliorating or minimizing one or more symptoms associated with the presence of the cancer or tumor.

The term “administer” and its grammatical equivalents as used herein refer to the act of delivering, or causing to be delivered, a therapeutic or a pharmaceutical composition to the body of a subject by a method described herein or otherwise known in the art. The therapeutic can be a compound, a polypeptide, an antibody, a cell, or a population of cells. Administering a therapeutic or a pharmaceutical composition includes prescribing a therapeutic or a pharmaceutical composition to be delivered into the body of a subject. Exemplary forms of administration include oral dosage forms, such as tablets, capsules, syrups, suspensions; injectable dosage forms, such as intravenous (IV), intramuscular (IM), or intraperitoneal (IP); transdermal dosage forms, including creams, jellies, powders, or patches; buccal dosage forms; inhalation powders, sprays, suspensions, and rectal suppositories.

The terms “effective amount,” “therapeutically effective amount,” and their grammatical equivalents as used herein refer to the administration of an agent to a subject, either alone or as a part of a pharmaceutical composition and either in a single dose or as part of a series of doses, in an amount that is capable of having any detectable, positive effect on any symptom, aspect, or characteristics of a disease, disorder or condition when administered to the subject. The therapeutically effective amount can be ascertained by measuring relevant physiological effects. The exact amount required vary from subject to subject, depending on the age, weight, and general condition of the subject, the severity of the condition being treated, the judgment of the clinician, and the like. An appropriate “effective amount” in any individual case can be determined by one of ordinary skill in the art using routine experimentation.

The term “pharmaceutically acceptable carrier” or “pharmaceutically acceptable excipient” refers to a material that is suitable for drug administration to an individual along with an active agent without causing undesirable biological effects or interacting in a deleterious manner with any of the other components of the pharmaceutical composition.

The term “subject” as used herein refers to any animal (e.g., a mammal), including, but not limited to, humans, non-human primates, canines, felines, rodents, and the like, which is to be the recipient of a particular treatment. A subject can be a human. A subject can have a particular disease or condition.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Exemplary genes and polypeptides are described herein with reference to GenBank numbers, GI numbers and/or SEQ ID NOS. It is understood that one skilled in the art can readily identify homologous sequences by reference to sequence sources, including but not limited to GenBank (ncbi.nlm.nih.gov/genbank/) and EMBL (embl.org/).

### 7.2 IL-2 Polypeptides

Provided herein are novel IL-2 polypeptides that are truncated, shuffled, and/or circularly permutated variants of the wildtype protein. The term “IL-2 polypeptide” and its grammatical equivalents as used herein refer to a polypeptide modified or derived from the wildtype IL-2 molecule. The IL-2 polypeptides can have different amino acid sequences but share certain structural features and functional properties with the wildtype IL-2. The changes to an amino acid sequence can be, for example, truncation, shuffling of functional domains, and/or circular permutation. The changes to an amino acid sequence can also be, for example, amino acid additions, deletions, or substitutions.

In some embodiments, an IL-2 polypeptide can be charactered in having amino acid mutations, such as additions, deletions, or substitutions compared to the wildtype IL-2. In some embodiments, an IL-2 polypeptide can be a truncated variant, or a subsequence, of the wildtype IL-2. For example, an IL-2 polypeptide can lack an N-terminal, C-terminal or internal fragment of wildtype IL-2. In some embodiments, an IL-2 polypeptide can have additional amino acid sequences not encompassed by the wildtype IL-2.

In some embodiments, an IL-2 polypeptide can be a shuffled variant of wildtype IL-2. For example, certain fragments or functional domains of wildtype IL-2 can be reordered or rearranged. The human wildtype IL-2 has 4 α-helixes regions. In some embodiments, an IL-2 polypeptide can have the 4 α-helixes reordered or rearranged and retain the basic structure and binding to receptors.

In some embodiments, an IL-2 polypeptide can be circularly permutated. As is known in the art, “circular permutation” refers to the process of fusing the N- and C-termini of a linear protein or polypeptide, directly or through a linker (using, e.g., recombinant methodologies) to form a circular molecule, and then cutting (or linearizing) the circular molecule at location different from the fusion point to form a new linear protein. Circular permutation preserves the sequence, structure, and function features of a protein or a polypeptide, while generating new C- and N-termini at different locations that results in desired properties.

One of ordinary skill in the art would appreciate that IL-2 polypeptides provided herein can include more than one type of modification mentioned above or otherwise known in the art. For example, an IL-2 polypeptide can be a truncated variant of wildtype IL-2 that contains amino acid substitutions. For another example, an IL-2 polypeptide can be a shuffled or circularly permutated variant of wildtype IL-2 that contains additional amino acid mutations such as additions, deletions or substitutions. In some embodiments, an IL-2 polypeptide can be a truncated variant of wildtype IL-2 that is further shuffled, and optionally further circularly permutated. The truncated, shuffled, and circularly permutated variant can optionally contain additional amino acid mutations such as additions, deletions and/or substitutions.

In some embodiments, the IL-2 polypeptides provided herein: (1) have reduced or abolished binding affinity to IL-2Rα compared to wildtype human IL-2; (2) have reduced or abolished binding affinity to IL-2Rαβγ compared to wildtype human IL-2; (3) bind to IL-2Rβγ with an affinity comparable to, equal to or greater than wildtype human IL-2's binding affinity for IL-2Rβγ; (4) activate IL-2Rβγ to an extent or amount comparable to, equal to or great than wildtype human IL-2 activates IL-2Rβγ; (5) do not preferentially bind to IL-2Rαβγ over IL-2Rβγ; (6) have comparable, equal, or greater potency in promoting the activation and/or expansion of immune effector cells (e.g., CD8+ T cells or NK cells) compared to wildtype IL-2; (7) have reduced or abolished function in promoting the activation and/or proliferation of regulatory T cells compared to wildtype IL-2; (8) can increase the ratio of immune effector cells (e.g., CD8+ T cells or NK cells) to regulatory cells (e.g., Treg cells); (9) have reduced toxicity in vivo; or (10) have negligible immunogenicity in human; or any combination of (1) to (10). In some embodiments, the IL-2 polypeptide provided herein have all functional properties mentioned above.

The IL-2 polypeptides provided herein have reduced binding to IL-2Rα compared to wildtype human IL-2. In some embodiments, the IL-2 polypeptides provided herein exhibit at least five-fold decrease in binding and/or potency in activating IL-2Rα as compared to wildtype human IL-2. In some embodiments, the IL-2 polypeptides exhibit at least 10-fold, 20-fold, 30-fold, 40-fold, 50-fold, 60-fold, 70-fold, 80-fold, 90-fold, or 100-fold decrease in binding and/or potency in activating IL-2Rα as compared to wildtype human IL-2. In some embodiments, the IL-2 polypeptides provided herein do not detectably bind IL-2Rα. In some embodiments, the IL-2 polypeptides provided herein do not detectably activate IL-2Rα.

In some embodiments, the IL-2 polypeptides provided herein have reduced or abolished binding affinity to IL-2Rαβγ compared to wildtype human IL-2. The IL-2 polypeptides provided herein can exhibit at least 10-fold, 20-fold, 30-fold, 40-fold, 50-fold, 60-fold, 70-fold, 80-fold, 90-fold, or 100-fold decrease in binding and/or potency in activating IL-2Rαβγ as compared to wildtype human IL-2. In some embodiments, the IL-2 polypeptides provided herein bind to IL-2Rαβγ with a KD that is at least 10-fold, 20-fold, 30-fold, 40-fold, 50-fold, 60-fold, 70-fold, 80-fold, 90-fold, or 100-fold higher compared to wildtype human IL-2. In some embodiments, the IL-2 polypeptides provided herein bind to IL-2Rαβγ with a K_{D} that is about 10-fold higher compared to wildtype human IL-2. In some embodiments, the IL-2 polypeptides provided herein do not preferentially bind to IL-2Rαβγ over IL-2Rβγ. In some embodiments, the IL-2 polypeptides provided herein bind to IL-2Rαβγ with an affinity that is comparable to its binding affinity to IL-2Rβγ. In some embodiments, the IL-2 polypeptides provided herein bind to IL-2Rαβγ with a K_{D} that is no less than 10%, no less than 20%, no less than 50%, or no less than 80% of the K_{D} with which it binds to IL-2Rβγ. In some embodiments, the IL-2 polypeptides provided herein bind to IL-2Rαβγ with a K_{D} that is no less than 50% of the K_{D} with which it binds to IL-2Rβγ.

In some embodiments, IL-2 polypeptides provided herein retain the ability of the wildtype protein to bind to and/or activate the IL-2Rβγ dimer. In some embodiments, IL-2 polypeptides provided herein bind to the IL-2Rβγ dimer with an affinity that is comparable to that of the wildtype IL-2. In some embodiments, the IL-2 polypeptides provided herein bind to human IL-2Rβγ dimer with a K_{D} between 1×10⁻¹¹ to 1×10⁻⁸ M. In some embodiments, the IL-2 polypeptides provided herein bind to human IL-2Rβγ dimer with a K_{D} between 1×10⁻¹¹ to 5×10⁻⁹ M, between 1×10⁻¹¹ to 1×10⁻⁹ M, between 1×10⁻¹¹ to 5×10⁻¹⁰ M, between 1×10⁻¹¹ to 1×10⁻¹⁰ M, between 1×10⁻¹⁰ to 1×10⁻⁸ M, between 1×10⁻¹⁰ to 5×10⁻⁹ M, or between 1×10⁻¹⁰ to 1×10⁻⁹ M. In some embodiments, the IL-2 polypeptides provided herein bind to human IL-2Rβγ dimer with a K_{D} that is about 1×10⁻¹¹ M, about 5×10⁻¹¹ M, about 1×10⁻¹⁰ M, about 5×10⁻¹⁰ M, about 1×10⁻⁹ M, about 5×10⁻⁹ M, or about 1×10⁻⁸ M. In some embodiments, the IL-2 polypeptides provided herein bind to human IL-2Rβγ dimer with a K_{D} that is about 1×10⁻¹⁰ M. In some embodiments, the IL-2 polypeptides provided herein bind to human IL-2Rβγ dimer with a K_{D} that is about 5×10⁻¹⁰ M. In some embodiments, the IL-2 polypeptides provided herein bind to human IL-2Rβγ dimer with a K_{D} that is about 1×10⁻⁹ M.

In some embodiments, IL-2 polypeptides provided herein activate IL-2Rβγ to an extent or amount comparable to, equal to or great than wildtype human IL-2 activates IL-2Rβγ. In some embodiments, IL-2 polypeptides provided herein retain the ability of the wildtype protein to activate the IL-2Rβγ dimer. In some embodiments, IL-2 polypeptides provided herein activate the IL-2Rβγ dimer with an EC50 that is comparable to that of the wildtype IL-2. In some embodiments, IL-2 polypeptides provided herein activate the IL-2Rβγ dimer with an EC50 that is no more than 20%, no more than 50%, no more than 100%, no more than 200%, no more than 300%, no more than 400%, or no more than 500% higher than the EC50 of the wildtype IL-2. In some embodiments, IL-2 polypeptides provided herein activate the IL-2Rβγ dimer with an EC50 that is no more than 100% higher than the EC50 of the wildtype IL-2.

Distinct from the wildtype IL-2, the IL-2 polypeptides provided herein selectively activate effector immune cells but not the regulatory cells. In some embodiments, IL-2 polypeptides provided herein have comparable, equal, or greater potency in promoting the activation and/or expansion of immune effector cells (e.g., CD8+ T cells or NK cells) compared to wildtype IL-2. In some embodiments, IL-2 polypeptides provided herein have reduced or abolished function in promoting the activation and/or proliferation of regulatory T cells compared to wildtype IL-2. In some embodiments, the EC50 of the IL-2 polypeptides provided herein in activating the CD8+ T cells or NK cells is comparable to that of the wildtype IL-2. In some embodiments, the IL-2 polypeptides provided herein activates the CD8+ T cells or NK cells in vitro with an EC5 that is about 1 nM, about 5 nM, about 10 nM, about 15 nM, about 20 nM, about 25 nM, about 30 M, about 35 nM, about 40 M, about 45 nM, about 50 nM, about 55 nM, about 60 nM, about 65 nM, about 70 nM, about 75 nM, about 80 M, about 85 nM, about 90 nM, or about 100 nM. In some embodiments, the IL-2 polypeptides provided herein activate the CD8+ T cells or NK cells in vitro with an EC5 that is about 5 nM. In some embodiments, the IL-2 polypeptides provided herein activate the CD8+ T cells or NK cells in vitro with an EC5 that is about 10 nM. In some embodiments, the IL-2 polypeptides provided herein activate the CD8+ T cells or NK cells in vitro with an EC5 that is about 20 nM. In some embodiments, the IL-2 polypeptides provided herein activate the CD8+ T cells or NK cells in vitro with an EC5 that is about 30 nM. In some embodiments, the IL-2 polypeptides provided herein activate the CD8+ T cells or NK cells in vitro with an EC5 that is about 50 nM.

In some embodiments, the EC50 of the IL-2 polypeptides provided herein in activating the regulatory T cells is at least 5-fold, at least 10-fold, at least 20-fold, at least 30-fold, at least 40-fold, at least 50-fold, at least 60-fold, at least 70-fold, at least 80-fold, at least 90-fold, or at least 100-fold higher comparable to that of the wildtype IL-2.

In some embodiments, the IL-2 polypeptides provided herein can increase the ratio of immune effector cells (e.g., CD8+ T cells or NK cells) to regulatory cells (e.g., Treg cells), or E:R ratio. In some embodiments, the IL-2 polypeptides provided herein can increase the E:R ratio in vivo. In some embodiments, the IL-2 polypeptides provided herein can increase the E:R ratio in vitro.

In some embodiments, the IL-2 polypeptides provided herein have reduced toxicity in vivo compared to wildtype IL-2. In some embodiments, the IL-2 polypeptides provided herein cause less or reduced side effect or adverse event in a subject as compared to wildtype IL-2. In some embodiments, the side effect or adverse event is reduced body weight. In some embodiments, side effect or adverse event is pulmonary edema.

In some embodiments, the IL-2 polypeptides provided herein do not have increased level of immunogenicity in human compared to wildtype human IL-2. In some embodiments, the IL-2 polypeptides provided herein have negligible level of immunogenicity in human.

Assays to measure the above-mentioned binding property, activity, or immunogenicity of an IL-2 polypeptide are well known in the art, some of which are described in the experimental section below. For example, a variety of methods of measuring binding affinity are known in the art, such as Western Blot, ELISA, Flow Cytometry, isothermal titration calorimetry (ITC), surface plasmon resonance (SPR), MicroScale Thermophoresis (MST), and Bio-Layer Interferometry (BLI). For example, a variety of methods can be used to measure the function of an IL-2 polypeptide in activating IL-2Rβ/γ or IL-2Rα, such as the STAT5 activation assay using cell lines expressing all IL-2Rα/β/γ (e.g., NK-92) or only IL-2Rβ/γ (e.g., HH cells). For example, a variety of assays can be used to measure the immunogenicity (or confirm the lack thereof) of an IL-2 polypeptide, such as by injection of the IL-2 polypeptide to non-human primates or humanized mouses. The specific type of T cell can be selectively stimulated. Besides, assays to measure the anti-tumor efficacy, the pharmacokinetics, pharmacodynamics, and/or toxicity of an IL-2 polypeptide are also well known in the art.

The wildtype human IL-2 can have the amino acid sequence of SEQ ID NO:19. Unprocessed human IL-2 additionally has an N-terminal 20-amino acid signal peptide, which is absent in the mature IL-2 molecule.

Various designations can be used to indicate the same mutation. The numbering of wildtype IL-2 is used herein to identify modifications of specific amino acid residues. For example, a mutation from cysteine at position 125 of the wildtype human IL-2 (SEQ ID NO:19) to serine can be indicated as C125S. For IL-2 polypeptides with different numbering (e.g., a circularly permutated variant or a shuffled variant), modifications can be designated with either its own numbering or the numbering of the wildtype. For example, if C64 in an IL-2 polypeptides corresponds to C125 of wildtype IL-2, modification of C64 of the specific polypeptides can be alternatively designated as modification of C125 according to the numbering of wildtype IL-2.

Wildtype IL-2 (SEQ ID NO: 19) has 4 α-helixes, which bind to IL-2Rβ and IL-2Rβγ, 2 loops that bind to IL-2Rα, and 4 linkers connecting the helixes and loops. The 133 amino acids of wildtype IL-2 can be divided into the following regions: Linker 1 (aa 1-5)—Helix 1 (aa 6-29)—Linker 2 (aa 30-34)—Loop 1 (aa 35-51)—Helix 2 (aa 52-71)—Linker 3 (aa 72-79)—Helix 3 (aa 80-98)—Linker 4 (aa 99-102)—Loop 2 (aa 103-112)—Helix 4 (aa 113-133).

TABLE 1
Sequences of wildtype IL-2

| | Resi- | |
|---|---|---|
| | dues | |
| Regions | (aa) | Sequences |
| Linker 1 | 1-5 | APTSS (SEQ ID NO: 1) |
| Helix 1 | 6-29 | STKKTQLQLEHLLLDLQMILNGIN |
| | | (SEQ ID NO: 2) |
| Linker 2 | 30-34 | NYKNP (SEQ ID NO: 3) |
| Loop 1 | 35-51 | KLTRMLTFKFYMPKKAT (SEQ ID |
| | | NO: 4) |
| Helix 2 | 52-71 | ELKHLQCLEEELKPLEEVLN (SEQ ID |
| | | NO: 5) |
| Linker 3 | 72-79 | LAQSKNFH (SEQ ID NO: 6) |
| Helix 3 | 80-98 | LRPRDLISNINVIVLELKG (SEQ ID |
| | | NO: 7) |
| Linker 4 | 99-102 | SETT (SEQ ID NO: 8) |
| Loop 2 | 103-112 | FMCEYADETA (SEQ ID NO: 9) |
| Helix 4 | 113-133 | TIVEFLNRWITFCQSIISTLT (SEQ |
| | | ID NO: 10) |

In some embodiments, the IL-2 polypeptides provided herein lack Loop 1. In some embodiments, the IL-2 polypeptides provided herein lack Loop 2. In some embodiments, the IL-2 polypeptides provided herein lack both Loop 1 and Loop 2.

In some embodiments, the IL-2 polypeptides provided herein have rearranged Helixes 1-4. In some embodiments, the IL-2 polypeptides provided herein comprise, from N-terminus to C-terminus, Helix 1, Helix 3, Helix 2, and Helix 4. In some embodiments, the IL-2 polypeptides provided herein comprise, from N-terminus to C-terminus, Helix 2, Helix 3, Helix 1, and Helix 4.

In some embodiments, provided herein are circularly permutated variants of IL-2 polypeptides provided herein. In some embodiments, provided herein are circularly permutated variants of wildtype IL-2. In some embodiments, provided herein are circularly permutated variants of IL-2 polypeptides disclosed herein. In some embodiments, provided herein are circularly permutated variants of a truncated IL-2. In some embodiments, provided herein are circularly permutated variants of a shuffled and truncated IL-2. In some embodiments, the cutting point of the circularly permutated variants is at the C-terminus of a helix region, namely, the C-terminus of H1, H3, H2, or H4. In some embodiments, the cutting point of the circularly permutated variants can be at any point of the IL-2 polypeptide as long as the variants maintain the basic structure and function to bind and activate IL-2Rβ/γ.

In some embodiments, provided herein are IL-2 polypeptides comprising, from N-terminus to C-terminus, (i) helix 1 (“H1”), helix 3 (“H3”), helix 2 (“H2”), and helix 4 (“H4”), or (ii) H2, H3, H1, and H4; or a circularly permutated variant thereof; wherein H1, H2, H3, and H4 corresponds to the Helix 1, Helix 2, Helix 3, and Helix 4 of wildtype IL-2, or a variant thereof. In some embodiments, H1, H2, H3, and H4 each have the amino acid sequences of SEQ ID NOs:2, 5, 7, and 10, respectively, or a variant thereof having up to five amino acid mutations.

In some embodiments, the IL-2 polypeptides provided herein comprise, from N-terminus to C-terminus, H1, H3, H2, and H4, or a circularly permutated variant thereof. In some embodiments, the IL-2 polypeptides comprise, from N-terminus to C-terminus, H1, H3, H2, and H4. In some embodiments, the cutting point of the circularly permutated variants is at the C-terminus of a helix region, namely, the C-terminus of H1, H3, H2, or H4. In some embodiments, the cutting point of the circularly permutated variants can be at any point of the IL-2 polypeptide as long as the variants maintain the basic structure and function to bind and activate IL-2Rβ/γ. In some embodiments, the circularly permutated IL-2 variants comprise, from N-terminus to C-terminus, H3, H2, H4, and H1. In some embodiments, the circularly permutated variants comprise, from N-terminus to C-terminus, H2, H4, H1, and H3. In some embodiments, the circularly permutated variants comprise, from N-terminus to C-terminus, H4, H1, H3, and H2.

In some embodiments, the IL-2 polypeptides provided herein comprise, from N-terminus to C-terminus, H2, H3, H1, and H4, or a circularly permutated variant thereof. In some embodiments, the IL-2 polypeptides comprise, from N-terminus to C-terminus, H2, H3, H1, and H4. In some embodiments, the cutting point of the circularly permutated variants is at the C-terminus of a helix region, namely, the C-terminus of H2, H3, H1, or H4. In some embodiments, the cutting point of the circularly permutated variants can be at any point of the IL-2 polypeptide as long as the variants maintain the basic structure and function to bind and activate IL-2Rβ/γ. In some embodiments, the circularly permutated variants comprise, from N-terminus to C-terminus, H3, H1, H4, and H2. In some embodiments, the circularly permutated variants comprise, from N-terminus to C-terminus, H1, H4, H2, and H3. In some embodiments, the circularly permutated variants comprise, from N-terminus to C-terminus, H4, H2, H3, and H1.

In some embodiments, the helix regions H1, H2, H3, and H4 of the IL-2 polypeptides provided herein each have the amino acid sequences of SEQ ID NOs:2, 5, 7, and 10, respectively, or a variant thereof having up to five amino acid mutations. The up to five amino acid mutations can be amino acid additions, deletions, and/or substitutions. The mutations can be conservation substitutions. In some embodiments, the IL-2 polypeptides maintain the basic structure of the helix regions and the binding to IL-2Rβ and IL-2Rβγ.

In some embodiments, the helix region H1 of the IL-2 polypeptides provided herein can have the amino acid sequence of STKKTQLQLEHLLLDLQMILNGIN (SEQ ID NO:2), or a variant thereof having up to five amino acid mutations. In some embodiments, H1 has the amino acid sequence of SEQ ID NO:2. In some embodiments, H1 can have an amino acid sequence that has up to three amino acid mutations compared to SEQ ID NO:2. In some embodiments, H1 can have an amino acid sequence that has two amino acid mutations compared to SEQ ID NO:2. In some embodiments, H1 can have an amino acid sequence that has one amino acid mutation compared to SEQ ID NO:2. In some embodiments, the H1 variant have amino acid mutations at E10, H11, L13, L14, or Q17; or any combination thereof of SEQ ID NO:2 (numbered according to residues of SEQ ID NO:2). The up to five amino acid mutations can be amino acid additions, deletions, and/or substitutions. In some embodiments, the mutations can be amino acid substitutions. In some embodiments, the mutations can be conservation substitutions.

In some embodiments, the helix region H2 of the IL-2 polypeptides provided herein can have the amino acid sequence of ELKHLQCLEEELKPLEEVLN (SEQ ID NO:5), or a variant thereof having up to five amino acid mutations. In some embodiments, H2 has the amino acid sequence of SEQ ID NO:5. In some embodiments, H2 can have an amino acid sequence that has up to three amino acid mutations compared to SEQ ID NO:5. In some embodiments, H2 has an amino acid sequence that has two amino acid mutations compared to SEQ ID NO:5. In some embodiments, H2 has an amino acid sequence that has one amino acid mutation compared to SEQ ID NO:5. In some embodiments, the H2 variant have amino acid mutations at C7, E10, P14, or E17; or any combination thereof of SEQ ID NO:5 (numbered according to residues of SEQ ID NO:5). The up to five amino acid mutations can be amino acid additions, deletions, and/or substitutions. In some embodiments, the mutations can be amino acid substitutions. In some embodiments, the mutations can be conservation substitutions. In some embodiments, H2 has a C7S substitution, with the amino acid sequence being ELKHLQSLEEELKPLEEVLN (SEQ ID NO:13). In some embodiments, H2 has a P14D substitution, with the amino acid sequence being ELKHLQCLEEELKDLEEVLN (SEQ ID NO:14). In some embodiments, H2 has both C7S and P14D substitutions, with the amino acid sequence being ELKHLQSLEEELKDLEEVLN (SEQ ID NO:15).

In some embodiments, the helix region H3 of the IL-2 polypeptides provided herein can have the amino acid sequence of LRPRDLISNINVIVLELKG (SEQ ID NO:7), or a variant thereof having up to five amino acid mutations. In some embodiments, H3 has the amino acid sequence of SEQ ID NO:7. In some embodiments, H3 can have an amino acid sequence that has up to three amino acid mutations compared to SEQ ID NO:7. In some embodiments, H3 can have an amino acid sequence that has two amino acid mutations compared to SEQ ID NO:7. In some embodiments, H3 can have an amino acid sequence that has one amino acid mutation compared to SEQ ID NO:7. In some embodiments, the H3 variant have amino acid mutations at S8, V12, L15, or E16; or any combination thereof of SEQ ID NO:7 (numbered according to residues of SEQ ID NO:7). The up to five amino acid mutations can be amino acid additions, deletions, and/or substitutions. In some embodiments, the mutations can be amino acid substitutions. In some embodiments, the mutations can be conservation substitutions.

In some embodiments, the helix region H4 of the IL-2 polypeptides provided herein can have the amino acid sequence of TIVEFLNRWITFCQSIISTLT (SEQ ID NO: 10), or a variant thereof having up to five amino acid mutations. In some embodiments, H4 has the amino acid sequence of SEQ ID NO: 10. In some embodiments, H4 can have an amino acid sequence that has up to three amino acid mutations compared to SEQ ID NO:10. In some embodiments, H4 can have an amino acid sequence that has two amino acid mutations compared to SEQ ID NO:10. In some embodiments, H4 can have an amino acid sequence that has one amino acid mutation compared to SEQ ID NO:10.

In some embodiments, the H4 variant have amino acid mutations at F5, N7, T11, F12, Q14, or S18; or any combination thereof of SEQ ID NO:10 (numbered according to residues of SEQ ID NO:10). The up to five amino acid mutations can be amino acid additions, deletions, and/or substitutions. In some embodiments, the mutations can be amino acid substitutions. In some embodiments, the mutations can be conservation substitutions. In some embodiments, H4 has a F5K substitution, with the amino acid sequence being TIVEKLNRWITFCQSIISTLT (SEQ ID NO: 16). In some embodiments, H4 has a F12K substitution, with the amino acid sequence being TIVEFLNRWITKCQSIISTLT (SEQ ID NO:17). In some embodiments, H4 has both F5K and F12K substitutions, with the amino acid sequence being TIVEKLNRWITKCQSIISTLT (SEQ ID NO:18).

In some embodiments of the IL-2 polypeptides provided herein: (1) H1 has the amino acid sequence of SEQ ID NO:2; (2) H2 has the amino acid sequence of SEQ ID NO:5, 13, 14, or 15; (3) H3 has the amino acid sequence of SEQ ID NO:7; or (4) H4 has the amino acid sequence of SEQ ID NO:10, 16, 17, or 18; or any combination thereof. In some embodiments, the IL-2 polypeptides provided herein have H1, H2, H3, and H4, having the amino acid sequences of (1) SEQ ID NOs:2, 5, 7, and 10, respectively; (2) SEQ ID NOs:2, 5, 7, and 16, respectively; (3) SEQ ID NOs:2, 5, 7, and 17, respectively; (4) SEQ ID NOs:2, 5, 7, and 18, respectively; (5) SEQ ID NOs:2, 13, 7, and 10, respectively; (6) SEQ ID NOs:2, 13, 7, and 16, respectively; (7) SEQ ID NOs:2, 13, 7, and 17, respectively; (8) SEQ ID NOs:2, 13, 7, and 18, respectively; (9) SEQ ID NOs:2, 14, 7, and 10, respectively; (10) SEQ ID NOs:2, 14, 7, and 16, respectively; (11) SEQ ID NOs:2, 14, 7, and 17, respectively; (12) SEQ ID NOs:2, 14, 7, and 18, respectively; (13) SEQ ID NOs:2, 15, 7, and 10, respectively; (14) SEQ ID NOs:2, 15, 7, and 16, respectively; (15) SEQ ID NOs:2, 15, 7, and 17, respectively; or (16) SEQ ID NOs:2, 15, 7, and 18, respectively.

As a person of ordinary skill in the art would appreciate, the helix regions of the IL-2 polypeptides provided herein can have further mutations or modifications to the specified amino acid sequences (e.g., SEQ ID NOs:2, 5, 7, and 10 for H1, H2, H3, and H4, respectively), so long as the mutations or modifications do not affect the basic structures and receptor binding function of the helix regions.

In some embodiments, the helix regions of the IL-2 polypeptides provided herein are connected by linkers. The linker can be a polypeptide sequence that connects the helix regions and/or other functional domains of the IL-2 polypeptides. A linker preserves some minimum distance or other spatial relationship between two sequences, and generally has no specific biological activity. The constituent amino acids of a linker can be selected based on some properties of the linker or of the resulting molecule such as the flexibility, hydrophilicity, net charge, or proteolytic susceptibility or lack thereof, and lack of immunogenicity. Some exemplary linkers that can be included in the IL-2 polypeptides disclosed herein are provided below. A person of ordinary skill in the art would understand that modifications can be made to these exemplary linkers and other peptide linkers known in the art can also be used.

In some embodiments, the IL-2 polypeptides provided herein further comprise (1) linker 1 (“L1”) located at the N-terminus of H1; (2) linker 2 (“L2”) located at (a) the C-terminus of H1; (3) linker 3 (“L3”) located at the C-terminus of H2; (4) linker 4 (“L4”) located at the C-terminus of H3; or (5) linker 5 (“L5”) located at C-terminus of H4; or any combination thereof; wherein L1, L2, L3, and L4 correspond to Linker 1, Linker 2, Linker 3, and Linker 4 of the wildtype IL-2, or a variant thereof. L5 can be an artificial linker. In some embodiments, either L2, L3, L4, or L5 is not included if the connecting helix is the ending helical region at C-terminus. For example, L2 is not needed if H1 is the ending helical region at C-terminus. L3 is not needed if H2 is the ending helical region at C-terminus. L4 is not needed if H3 is the ending helical region at C-terminus. L5 is not needed if H4 is the ending helical region at C-terminus. As such, in some embodiments, IL-2 polypeptides provided herein comprise (1) L1; (2) L2, except when H1 is located at the C-terminus of the IL-2 polypeptide; (3) L3, except when H2 is located at the C-terminus of the IL-2 polypeptide; (4) L4, except when H3 is located at the C-terminus of the IL-2 polypeptide; and (5) L5, except when H4 is located at the C-terminus of the IL-2 polypeptide.

In some embodiments, L1, L2, L3, and L4 each have the amino acid sequences of SEQ ID NOs:1, 3, 6, and 8, respectively, or a variant thereof having up to three amino acid mutations. In some embodiments, L5 can have the amino acid sequence of (GGGGS)n, n=1, 2, 3, 4, or 5 (SEQ ID NO:44). In some embodiments, the linker has the amino acid sequence of (EAAAK)n, n=1, 2, 3, 4, or 5 (SEQ ID NO:45). In some embodiments, the linker has the amino acid sequence of (PA)nP, n=1, 2, 3, 4, or 5 (SEQ ID NO:46). In some embodiments, the linker has the amino acid sequence of GGGGSGGGGS (SEQ ID NO:11).

In some embodiments of the IL-2 polypeptides provided herein, (1) L1 has the amino acid sequence of SEQ ID NO:1 or 12; (2) L2 has the amino acid sequence of SEQ ID NO:3; (3) L3 has the amino acid sequence of SEQ ID NO:6; (4) L4 has the amino acid sequence of SEQ ID NO:8; (5) L5 has the amino acid sequence of SEQ ID NO: 11; or any combination thereof.

In some embodiments, the linker L1 of the IL-2 polypeptides provided herein can have the amino acid sequence of SEQ ID NO:1, or a variant thereof having up to three amino acid mutations. In some embodiments, L1 has the amino acid sequence of SEQ ID NO:1. In some embodiments, L1 can have an amino acid sequence that has two amino acid mutations compared to SEQ ID NO:1. In some embodiments, L1 can have an amino acid sequence that has one amino acid mutation compared to SEQ ID NO:1. In some embodiments, L1 has the amino acid sequence of PTSS (SEQ ID NO:12).

In some embodiments, the linker L2 of the IL-2 polypeptides provided herein can have the amino acid sequence of SEQ ID NO:3, or a variant thereof having up to three amino acid mutations. In some embodiments, L2 has the amino acid sequence of SEQ ID NO:3. In some embodiments, L2 can have an amino acid sequence that has two amino acid mutations compared to SEQ ID NO:3. In some embodiments, L2 can have an amino acid sequence that has one amino acid mutation compared to SEQ ID NO:3.

In some embodiments, the linker L3 of the IL-2 polypeptides provided herein can have the amino acid sequence of SEQ ID NO:6, or a variant thereof having up to three amino acid mutations. In some embodiments, L3 has the amino acid sequence of SEQ ID NO:6. In some embodiments, L3 can have an amino acid sequence that has two amino acid mutations compared to SEQ ID NO:6. In some embodiments, L3 can have an amino acid sequence that has one amino acid mutation compared to SEQ ID NO:6.

In some embodiments, the linker L4 of the IL-2 polypeptides provided herein can have the amino acid sequence of SEQ ID NO:8, or a variant thereof having up to three amino acid mutations. In some embodiments, L4 has the amino acid sequence of SEQ ID NO:8. In some embodiments, L4 can have an amino acid sequence that has two amino acid mutations compared to SEQ ID NO:8. In some embodiments, L4 can have an amino acid sequence that has one amino acid mutation compared to SEQ ID NO:8.

Additional mutations can be introduced to IL-2 polypeptides provided herein (e.g., the circularly permutated, shuffled, and truncated IL-2 variants disclosed herein) for various purposes, such as to increase expression, improve stability, enhance activity, etc, which can be confirmed using the functional assays known in the art. Any combination of substitution, deletion, insertion, and modification can be made to arrive at the final construct, provided that the final construct possesses the desired characteristics, e.g., reduced binding to IL-2Ra. Amino acid substitutions include replacement by non-naturally occurring amino acids or by naturally occurring amino acid derivatives of the twenty standard amino acids (e.g., 4-hydroxyproline, 3-methylhistidine, ornithine, homoserine, 5-hydroxylysine). Amino acid mutations can be generated using genetic or chemical methods well known in the art. Genetic methods can include site-directed mutagenesis, PCR, gene synthesis, and the like. It is contemplated that methods of altering the side chain group of an amino acid by methods other than genetic engineering, e.g., chemical modification, can also be useful.

For example, in some embodiments, IL-2 polypeptides provided herein lack the first alanine residue of wildtype IL-2, referred to as de-alanine-1. In some embodiments, IL-2 polypeptides provided herein comprise a mutation at C58 according to the numbering of the wildtype IL-2 (or C7 of Helix 2, SEQ ID NO:5). In some embodiments, the mutation is C58S substitution. In some embodiments, IL-2 polypeptides provided herein comprise a mutation of C125 according to the numbering of the wildtype IL-2 (or C13 of Helix 4, SEQ ID NO: 10). In some embodiments, the mutation is C125S substitution.

The IL-2 polypeptides provided herein can have additional mutations at the helix regions to further stabilize the helix. In some embodiments, the hydrophobic residues of the IL-2 polypeptides can be mutated to decrease the hydrophobicity of surface. In some embodiments, IL-2 polypeptides provided herein comprise a mutation at F117 according to the numbering of the wildtype IL-2 (or F5 of Helix 4, SEQ ID NO:10). In some embodiments, IL-2 polypeptides provided herein comprise a mutation at F124 according to the numbering of wildtype IL-2 (or F12 of Helix 4, SEQ ID NO:10). In some embodiments, IL-2 polypeptides provided herein comprise mutations at both F117 and F124 according to the numbering of the wildtype IL-2. The mutation can be amino acid substitution. In some embodiments, the mutation can be basic mutation, such as F117K, F124K, or both F117K and F124K.

In some embodiments, the IL-2 polypeptides can further include one or more conservative modifications. Such conservative modifications include amino acid substitutions, additions, and deletions. Modifications can be introduced by standard techniques known in the art, such as site-directed mutagenesis and PCR-mediated mutagenesis. In some embodiments, the conservative modifications do not significantly affect or alter the desired functional properties of the polypeptide Conservative amino acid substitutions include those in which the amino acid residue is replaced with an amino acid residue having a similar side chain. Families of amino acid residues having similar side chains have been defined in the art, including: amino acids with basic side chains (e.g., lysine, arginine, histidine); acidic side chains (e.g., aspartic acid, glutamic acid); uncharged polar side chains (e.g., glycine, asparagine, glutamine, serine, threonine, tyrosine, cysteine, tryptophan); nonpolar side chains (e.g., alanine, valine, leucine, isoleucine, proline, phenylalanine, methionine); beta-branched side chains (e.g., threonine, valine, isoleucine); and aromatic side chains (e.g., tyrosine, phenylalanine, tryptophan, histidine) includes one or more conservative modifications.

Some IL-2 polypeptides are provided below, which are intended to exemplify the IL-2 polypeptides provided herein, and not to be limiting.

TABLE 2
Exemplary IL-2 polypeptides

| CP-trIL-2 | Amino Acid Sequences |
|---|---|
| 001 (Wildtype) | APTSSSTKKTQLQLEHLLLDLQMILNGINNYKNPKLTRMLTFKFYMP |
| | KKATELKHLQCLEEELKPLEEVLNLAQSKNFHLRPRDLISNINVIVL |
| | ELKGSETTFMCEYADETATIVEFLNRWITFCQSIISTLT (SEQ ID |
| | NO: 19) |
| 011 | PTSSSTKKTQLQLEHLLLDLQMILNGINNYKNPLRPRDLISNINVIV |
| | LELKGSETTELKHLQSLEEELKPLEEVLNLAQSKNFHTIVEFLNRWI |
| | TFSQSIISTLT (SEQ ID NO: 20) |
| 012 | PTSSSTKKTQLQLEHLLLDLQMILNGINNYKNPLRPRDLISNINVIV |
| | LELKGSETTELKHLQSLEEELKDLEEVLNLAQSKNFHTIVEFLNRWI |
| | TFSQSIISTLT (SEQ ID NO: 21) |
| 013 | PTSSSTKKTQLQLEHLLLDLQMILNGINNYKNPLRPRDLISNINVIV |
| | LELKGSETTELKHLQSLEEELKDLEEVLNLAQSKNFHTIVEKLNRWI |
| | TKSQSIISTLT (SEQ ID NO: 22) |
| 021 | LRPRDLISNINVIVLELKGSETTELKHLQSLEEELKPLEEVLNLAQS |
| | KNFHTIVEFLNRWITESQSIISTLTGGGGSGGGGSPTSSSTKKTQLQ |
| | LEHLLLDLQMILNGIN (SEQ ID NO: 23) |
| 022 | LRPRDLISNINVIVLELKGSETTELKHLQSLEEELKDLEEVLNLAQS |
| | KNFHTIVEFLNRWITFSQSIISTLTGGGGSGGGGSPTSSSTKKTQLQ |
| | LEHLLLDLQMILNGIN (SEQ ID NO: 24) |
| 023 | LRPRDLISNINVIVLELKGSETTELKHLQSLEEELKDLEEVLNLAQS |
| | KNFHTIVEKLNRWITKSQSIISTLTGGGGSGGGGSPTSSSTKKTQLQ |
| | LEHLLLDLQMILNGIN (SEQ ID NO: 25) |
| 031 | ELKHLQSLEEELKPLEEVLNLAQSKNFHTIVEFLNRWITFSQSIIST |
| | LTGGGGSGGGGSPTSSSTKKTQLQLEHLLLDLQMILNGINNYKNPLR |
| | PRDLISNINVIVLELKG (SEQ ID NO: 26) |
| 032 | ELKHLQSLEEELKDLEEVLNLAQSKNFHTIVEFLNRWITFSQSIIST |
| | LTGGGGSGGGGSPTSSSTKKTQLQLEHLLLDLQMILNGINNYKNPLR |
| | PRDLISNINVIVLELKG (SEQ ID NO: 27) |
| 033 | ELKHLQSLEEELKDLEEVLNLAQSKNFHTIVEKLNRWITKSQSIIST |
| | LTGGGGSGGGGSPTSSSTKKTQLQLEHLLLDLQMILNGINNYKNPLR |
| | PRDLISNINVIVLELKG (SEQ ID NO: 28) |
| 041 | TIVEFLNRWITFSQSIISTLTGGGGSGGGGSPTSSSTKKTQLQLEHL |
| | LLDLQMILNGINNYKNPLRPRDLISNINVIVLELKGSETTELKHLQS |
| | LEEELKPLEEVLN (SEQ ID NO: 29) |
| 042 | TIVEFLNRWITFSQSIISTLTGGGGSGGGGSPTSSSTKKTQLQLEHL |
| | LLDLQMILNGINNYKNPLRPRDLISNINVIVLELKGSETTELKHLQS |
| | LEEELKDLEEVLN (SEQ ID NO: 30) |
| 043 | TIVEKLNRWITKSQSIISTLTGGGGSGGGGSPTSSSTKKTQLQLEHL |
| | LLDLQMILNGINNYKNPLRPRDLISNINVIVLELKGSETTELKHLQS |
| | LEEELKDLEEVLN (SEQ ID NO: 31) |
| 051 | PTSSSTKKTQLQLEHLLLDLQMILNGINNYKNPTIVEFLNRWITFSQ |
| | SIISTLTGGGGSGGGGSELKHLQSLEEELKPLEEVLNLAQSKNFHLR |
| | PRDLISNINVIVLELKG (SEQ ID NO: 32) |
| 052 | PTSSSTKKTQLQLEHLLLDLQMILNGINNYKNPTIVEFLNRWITFSQ |
| | SIISTLTGGGGSGGGGSELKHLQSLEEELKDLEEVLNLAQSKNFHLR |
| | PRDLISNINVIVLELKG (SEQ ID NO: 33) |
| 053 | PTSSSTKKTQLQLEHLLLDLQMILNGINNYKNPTIVEKLNRWITKSQ |
| | SIISTLTGGGGSGGGGSELKHLQSLEEELKDLEEVLNLAQSKNFHLR |
| | PRDLISNINVIVLELKG (SEQ ID NO: 34) |
| 061 | TIVEFLNRWITFSQSIISTLTGGGGSGGGGSELKHLQSLEEELKPLE |
| | EVLNLAQSKNFHLRPRDLISNINVIVLELKGSETTPTSSSTKKTQLQ |
| | LEHLLLDLQMILNGIN (SEQ ID NO: 35) |
| 062 | TIVEFLNRWITFSQSIISTLTGGGGGGGGSELKHLQSLEEELKDLE |
| | EVLNLAQSKNFHLRPRDLISNINVIVLELKGSETTPTSSSTKKTQLQ |
| | LEHLLLDLQMILNGIN (SEQ ID NO: 36) |
| 063 | TIVEKLNRWITKSQSIISTLTGGGGSGGGGSELKHLQSLEEELKDLE |
| | EVLNLAQSKNFHLRPRDLISNINVIVLELKGSETTPTSSSTKKTQLQ |
| | LEHLLLDLQMILNGIN (SEQ ID NO: 37) |
| 071 | ELKHLQSLEEELKPLEEVLNLAQSKNFHLRPRDLISNINVIVLELKG |
| | SETTPTSSSTKKTQLQLEHLLLDLQMILNGINNYKNPTIVEFLNRWI |
| | TFSQSIISTLT (SEQ ID NO: 38) |
| 072 | ELKHLQSLEEELKDLEEVLNLAQSKNFHLRPRDLISNINVIVLELKG |
| | SETTPTSSSTKKTQLQLEHLLLDLQMILNGINNYKNPTIVEFLNRWI |
| | TFSQSIISTLT (SEQ ID NO: 39) |
| 073 | ELKHLQSLEEELKDLEEVLNLAQSKNFHLRPRDLISNINVIVLELKG |
| | SETTPTSSSTKKTQLQLEHLLLDLQMILNGINNYKNPTIVEKLNRWI |
| | TKSQSIISTLT (SEQ ID NO: 40) |
| 081 | LRPRDLISNINVIVLELKGSETTPTSSSTKKTQLQLEHLLLDLQMIL |
| | NGINNYKNPTIVEFLNRWITFSQSIISTLTGGGGSGGGGSELKHLQS |
| | LEEELKPLEEVLN (SEQ ID NO: 41) |
| 082 | LRPRDLISNINVIVLELKGSETTPTSSSTKKTQLQLEHLLLDLQMIL |
| | NGINNYKNPTIVEFLNRWITFSQSIISTLTGGGGSGGGGSELKHLQS |
| | LEEELKDLEEVLN (SEQ ID NO: 42) |
| 083 | LRPRDLISNINVIVLELKGSETTPTSSSTKKTQLQLEHLLLDLQMIL |
| | NGINNYKNPTIVEKLNRWITKSQSIISTLTGGGGGGGGSELKHLQS |
| | LEEELKDLEEVLN (SEQ ID NO: 43) |

In some embodiments, the IL-2 polypeptides provided herein comprising from N-terminus to C-terminus, H1, H3, H2, and H4, can further comprise linkers connecting the helix regions. In some embodiments, the IL-2 polypeptides comprise from N-terminus to C-terminus, L1, H1, L2, H3, L4, H2, L3, and H4. In some embodiments, the IL-2 polypeptides provided herein can have an amino acid sequence that is at least 85%, at least 90%, at least 95% at least 96%, at least 97% at least 98%, or at least 99% identical to SEQ ID NO:20. In some embodiments, the IL-2 polypeptides provided herein can have an amino acid sequence that is at least 85% identical to SEQ ID NO:20. In some embodiments, the IL-2 polypeptides provided herein can have an amino acid sequence that is at least 90% identical to SEQ ID NO:20. In some embodiments, the IL-2 polypeptides provided herein can have an amino acid sequence that is at least 95% identical to SEQ ID NO:20. In some embodiments, the IL-2 polypeptides provided herein can have an amino acid sequence that is at least 99% identical to SEQ ID NO:20. In some embodiments, the IL-2 polypeptides provided herein can have an amino acid sequence that is SEQ ID NO:20, 21, or 22. In some embodiments, the IL-2 polypeptides provided herein can have the amino acid sequence of SEQ ID NO:20. In some embodiments, the IL-2 polypeptides provided herein can have the amino acid sequence of SEQ ID NO:21. In some embodiments, the IL-2 polypeptides provided herein can have the amino acid sequence of SEQ ID NO:22.

In some embodiments, the IL-2 polypeptides provided herein comprising from N-terminus to C-terminus, H3, H2, H4, and H1, can further comprise linkers connecting the helix regions. In some embodiments, the IL-2 polypeptides comprise from N-terminus to C-terminus, H3, L4, H2, L3, H4, L5, L1, and H1. In some embodiments, the IL-2 polypeptides provided herein can have an amino acid sequence that is at least 85%, at least 90%, at least 95%, at least 96%, at least 97%, at least 98%, or at least 99% identical to SEQ ID NO:23. In some embodiments, the IL-2 polypeptides provided herein can have an amino acid sequence that is at least 85% identical to SEQ ID NO:23. In some embodiments, the IL-2 polypeptides provided herein can have an amino acid sequence that is at least 90% identical to SEQ ID NO:23. In some embodiments, the IL-2 polypeptides provided herein can have an amino acid sequence that is at least 95% identical to SEQ ID NO:23. In some embodiments, the IL-2 polypeptides provided herein can have an amino acid sequence that is at least 99% identical to SEQ ID NO:23. In some embodiments, the IL-2 polypeptides provided herein can have an amino acid sequence that is SEQ ID NO:23, 24, or 25. In some embodiments, the IL-2 polypeptides provided herein can have the amino acid sequence of SEQ ID NO:23. In some embodiments, the IL-2 polypeptides provided herein can have the amino acid sequence of SEQ ID NO:24. In some embodiments, the IL-2 polypeptides provided herein can have the amino acid sequence of SEQ ID NO:25.

In some embodiments, the IL-2 polypeptides provided herein comprising from N-terminus to C-terminus, H2, H4, H1, and H3, can further comprise linkers connecting the helix regions. In some embodiments, the IL-2 polypeptides comprise from N-terminus to C-terminus, H2, L3, H4, L5, L1, H1, L2, and H3. In some embodiments, the IL-2 polypeptides provided herein can have an amino acid sequence that is at least 85%, at least 90%, at least 95%, at least 96%, at least 97%, at least 98%, or at least 99% identical to SEQ ID NO:26. In some embodiments, the IL-2 polypeptides provided herein can have an amino acid sequence that is at least 85% identical to SEQ ID NO:26. In some embodiments, the IL-2 polypeptides provided herein can have an amino acid sequence that is at least 90% identical to SEQ ID NO:26. In some embodiments, the IL-2 polypeptides provided herein can have an amino acid sequence that is at least 95% identical to SEQ ID NO:26. In some embodiments, the IL-2 polypeptides provided herein can have an amino acid sequence that is at least 99% identical to SEQ ID NO:26. In some embodiments, the IL-2 polypeptides provided herein can have an amino acid sequence that is SEQ ID NO:26, 27, or 28. In some embodiments, the IL-2 polypeptides provided herein can have the amino acid sequence of SEQ ID NO:26. In some embodiments, the IL-2 polypeptides provided herein can have the amino acid sequence of SEQ ID NO:27. In some embodiments, the IL-2 polypeptides provided herein can have the amino acid sequence of SEQ ID NO:28.

In some embodiments, the IL-2 polypeptides provided herein comprising from N-terminus to C-terminus, H4, H1, H3, and H2, can further comprise linkers connecting the helix regions. In some embodiments, the IL-2 polypeptides comprise from N-terminus to C-terminus, H4, L5, L1, H1, L2, H3, L4, and H2. In some embodiments, the IL-2 polypeptides provided herein can have an amino acid sequence that is at least 85%, at least 90%, at least 95%, at least 96%, at least 97%, at least 98%, or at least 99% identical to SEQ ID NO:29. In some embodiments, the IL-2 polypeptides provided herein can have an amino acid sequence that is at least 85% identical to SEQ ID NO:29. In some embodiments, the IL-2 polypeptides provided herein can have an amino acid sequence that is at least 90% identical to SEQ ID NO:29. In some embodiments, the IL-2 polypeptides provided herein can have an amino acid sequence that is at least 95% identical to SEQ ID NO:29. In some embodiments, the IL-2 polypeptides provided herein can have an amino acid sequence that is at least 99% identical to SEQ ID NO:29. In some embodiments, the IL-2 polypeptides provided herein can have an amino acid sequence that is SEQ ID NO:29, 30, or 31. In some embodiments, the IL-2 polypeptides provided herein can have the amino acid sequence of SEQ ID NO:29. In some embodiments, the IL-2 polypeptides provided herein can have the amino acid sequence of SEQ ID NO:30. In some embodiments, the IL-2 polypeptides provided herein can have the amino acid sequence of SEQ ID NO:31.

In some embodiments, the IL-2 polypeptides provided herein comprising from N-terminus to C-terminus, H1, H4, H2, and H3, can further comprise linkers connecting the helix regions. In some embodiments, the IL-2 polypeptides comprise from N-terminus to C-terminus, L1, H1, L2, H4, L5, H2, L3, and H3. In some embodiments, the IL-2 polypeptides provided herein can have an amino acid sequence that is at least 85%, at least 90%, at least 95%, at least 96%, at least 97%, at least 98%, or at least 99% identical to SEQ ID NO:32. In some embodiments, the IL-2 polypeptides provided herein can have an amino acid sequence that is at least 85% identical to SEQ ID NO:32. In some embodiments, the IL-2 polypeptides provided herein can have an amino acid sequence that is at least 90% identical to SEQ ID NO:32. In some embodiments, the IL-2 polypeptides provided herein can have an amino acid sequence that is at least 95% identical to SEQ ID NO:32. In some embodiments, the IL-2 polypeptides provided herein can have an amino acid sequence that is at least 99% identical to SEQ ID NO:32. In some embodiments, the IL-2 polypeptides provided herein can have an amino acid sequence that is SEQ ID NO:32, 33, or 34. In some embodiments, the IL-2 polypeptides provided herein can have the amino acid sequence of SEQ ID NO:32. In some embodiments, the IL-2 polypeptides provided herein can have the amino acid sequence of SEQ ID NO:33. In some embodiments, the IL-2 polypeptides provided herein can have the amino acid sequence of SEQ ID NO:34.

In some embodiments, the IL-2 polypeptides provided herein comprising from N-terminus to C-terminus, H4, H2, H3, and H1, can further comprise linkers connecting the helix regions. In some embodiments, the IL-2 polypeptides comprise from N-terminus to C-terminus, H4, L5, H2, L3, H3, L4, L1, and H1. In some embodiments, the IL-2 polypeptides provided herein can have an amino acid sequence that is at least 85%, at least 90%, at least 95%, at least 96%, at least 97%, at least 98%, or at least 99% identical to SEQ ID NO:35. In some embodiments, the IL-2 polypeptides provided herein can have an amino acid sequence that is at least 85% identical to SEQ ID NO:35. In some embodiments, the IL-2 polypeptides provided herein can have an amino acid sequence that is at least 90% identical to SEQ ID NO:35. In some embodiments, the IL-2 polypeptides provided herein can have an amino acid sequence that is at least 95% identical to SEQ ID NO:35. In some embodiments, the IL-2 polypeptides provided herein can have an amino acid sequence that is at least 99% identical to SEQ ID NO:35. In some embodiments, the IL-2 polypeptides provided herein can have an amino acid sequence that is SEQ ID NO:35, 36, or 37. In some embodiments, the IL-2 polypeptides provided herein can have the amino acid sequence of SEQ ID NO:35. In some embodiments, the IL-2 polypeptides provided herein can have the amino acid sequence of SEQ ID NO:36. In some embodiments, the IL-2 polypeptides provided herein can have the amino acid sequence of SEQ ID NO:37.

In some embodiments, the IL-2 polypeptides provided herein comprising from N-terminus to C-terminus, H2, H3, H1, and H4, can further comprise linkers connecting the helix regions. In some embodiments, the IL-2 polypeptides comprise from N-terminus to C-terminus, H2, L3, H3, L4, L1, H1, L2, and H4. In some embodiments, the IL-2 polypeptides provided herein can have an amino acid sequence that is at least 85%, at least 90%, at least 95%, at least 96%, at least 97%, at least 98%, or at least 99% identical to SEQ ID NO:38. In some embodiments, the IL-2 polypeptides provided herein can have an amino acid sequence that is at least 85% identical to SEQ ID NO:38. In some embodiments, the IL-2 polypeptides provided herein can have an amino acid sequence that is at least 90% identical to SEQ ID NO:38. In some embodiments, the IL-2 polypeptides provided herein can have an amino acid sequence that is at least 95% identical to SEQ ID NO:38. In some embodiments, the IL-2 polypeptides provided herein can have an amino acid sequence that is at least 99% identical to SEQ ID NO:38. In some embodiments, the IL-2 polypeptides provided herein can have an amino acid sequence that is SEQ ID NO:38, 39, or 40. In some embodiments, the IL-2 polypeptides provided herein can have the amino acid sequence of SEQ ID NO:38. In some embodiments, the IL-2 polypeptides provided herein can have the amino acid sequence of SEQ ID NO:39. In some embodiments, the IL-2 polypeptides provided herein can have the amino acid sequence of SEQ ID NO:40.

In some embodiments, the IL-2 polypeptides provided herein comprising from N-terminus to C-terminus, H3, H1, H4, and H2, can further comprise linkers connecting the helix regions. In some embodiments, the IL-2 polypeptides comprise from N-terminus to C-terminus, H3, L4, L1, H1, L2, H4, L5, and H2. In some embodiments, the IL-2 polypeptides provided herein can have an amino acid sequence that is at least 85%, at least 90%, at least 95%, at least 96%, at least 97%, at least 98%, or at least 99% identical to SEQ ID NO:41. In some embodiments, the IL-2 polypeptides provided herein can have an amino acid sequence that is at least 85% identical to SEQ ID NO:41. In some embodiments, the IL-2 polypeptides provided herein can have an amino acid sequence that is at least 90% identical to SEQ ID NO:41. In some embodiments, the IL-2 polypeptides provided herein can have an amino acid sequence that is at least 95% identical to SEQ ID NO:41. In some embodiments, the IL-2 polypeptides provided herein can have an amino acid sequence that is at least 99% identical to SEQ ID NO:41. In some embodiments, the IL-2 polypeptides provided herein can have an amino acid sequence that is SEQ ID NO:41, 42, or 43. In some embodiments, the IL-2 polypeptides provided herein can have the amino acid sequence of SEQ ID NO:41. In some embodiments, the IL-2 polypeptides provided herein can have the amino acid sequence of SEQ ID NO:42. In some embodiments, the IL-2 polypeptides provided herein can have the amino acid sequence of SEQ ID NO:43.

In some embodiments, the IL-2 polypeptides provided herein can include modifications such as phosphorylation, glycosylation, ubiquitination, nitrosylation, methylation, acetylation, lipidation, and combinations thereof.

In some embodiments, the IL-2 polypeptides provided herein can be conjugated or linked to a detectable tag or a detectable marker (e.g., a radionuclide, a fluorescent dye, or an MRI-detectable label). In some embodiments, the detectable tag can be an affinity tag. The term “affinity tag” as used herein relates to a moiety attached to a polypeptide, which allows the polypeptide to be purified from a biochemical mixture. Affinity tags can consist of amino acid sequences or can include amino acid sequences to which chemical groups are attached by post-translational modifications. Non-limiting examples of affinity tags include His-tag, CBP-tag (CBP: calmodulin binding protein), CYD-tag (CYD: covalent yet dissociable NorpD peptide), Strep-tag, StrepII-tag, FLAG-tag, HPC-tag (HPC: heavy chain of protein C), GST-tag (GST: glutathione S transferase), Avi-tag, biotinylated tag, Myc-tag, a myc-myc-hexahistidine (mmh) tag 3xFLAG tag, a SUMO tag, and MBP-tag (MBP: maltose-binding protein). Further examples of affinity tags are known in the art.

In some embodiments, the detectable tag can be conjugated or linked to the N- and/or C-terminus of the IL-2 polypeptides. The detectable tag and the affinity tag can also be separated by one or more amino acids. In some embodiments, the detectable tag can be conjugated or linked to the IL-2 polypeptide via a cleavable element, which can be peptide sequences that are susceptible to cleavage by chemical agents or enzyme means, such as proteases. Proteases can be sequence-specific (e.g., thrombin) or have limited sequence specificity (e.g., trypsin). Cleavable elements I and II can also be included in the amino acid sequence of a detection tag or polypeptide, particularly where the last amino acid of the detection tag or polypeptide is K or R.

In some embodiments, the IL-2 polypeptides provided herein can be linked to IL-2 receptor alpha, optionally by a disulfide bond.

In some embodiments, the IL-2 polypeptides provided herein can be conjugated or linked to at least one polymer (e.g., polyethylene glycol (PEG)), sugar moiety (e.g., N-acetyl galactosamine (GalNAc), triantennary GalNAc), antibody or a fragment thereof (e.g., Fc domain), drug molecule (e.g., anti-cancer or anti-turn or drugs).

In some embodiments, the IL-2 polypeptides provided herein can be fused to albumin (e.g., human serum albumin (HSA)) or fragments or variants of albumin, or a molecule capable of binding HSA (an “anti-HSA binder”). In some embodiments, the anti-HSA binder can be an antibody or antibody fragment. In some embodiments, the anti-HSA binder can be a Fab, an scFv, or a VHH. Human serum albumin possesses many desirable characteristics. HSA is found throughout the body and has favorable pharmacokinetic properties. It is cleared very slowly by the liver displaying in vivo half-lives up to several weeks. HSA lacks enzymatic activity and antigenicity thereby eliminating potentially undesirable side effects. Thus, HSA can act as a carrier for the IL-2 polypeptides provided herein, and stabilize the protein and/or its activity, in vitro and/or in vivo.

In some embodiments, the IL-2 polypeptides provided herein can be conjugated to one or more polyethylene glycol (PEG). PEGylation is a method well known to those skilled in the art wherein a polypeptide is modified such that one or more polyethylene glycol (PEG) molecules are covalently attached to the side chain of one or more amino acids or derivatives thereof, which can improve the pharmacodynamic properties of the molecule, for example extending its half-life in vivo. A PEG-IL-2 polypeptides conjugate is formed by first activating the PEG moiety so that it will react with, and couple to, the IL-2 polypeptides provided herein. Methods that can be used to covalently attach the PEG molecules to polypeptides are well known in the art.

In some embodiments, the IL-2 polypeptides provided herein can be conjugated to a lipid moiety. For lipidation, a lipid chain can be a C12 to C20 lipid chain. In some embodiments, a lipid chain can be a C16 chain (e.g., a palmitic acid) or C18 chain (e.g., a stearic acid). The IL-2 polypeptides can be lipidated by any conventional or acceptable method known in the art to introduce lipids to peptides. This can be achieved by attaching one or more lipid amities to the peptides. There are several ways for introducing lipids. The lipids can be attached via an oligopeptide spacer at either the N- or C-terminus of the peptides between the peptide and the lipid moiety. The oligopeptide can comprise any number of amino acid residues and the lipid moiety can be attached to any of the amino acid residues in the oligopeptide. The lipid moiety can be bulky and can be added to the N-terminal end of the oligopeptide such that it is separated from the amino acids of the peptide to prevent any possible interference with functional portions, for example, of the amino acids in the cyclic peptides. A suitable spacer can be selected for the particular application used. The IL-2 polypeptides provided herein incorporating lipidation can contain one or more lipid moieties, for example, two lipid moieties per peptide.

In some embodiments, the IL-2 polypeptides provided herein can be fused to a moiety for targeting tumor cells. In some embodiments, such moiety can bind to a tumor-associated antigen (e.g., PSMA, MUC16); bind to a tumor microenvironment antigen (ECM); binds to a cell surface molecule of tumor-reactive lymphocytes (e.g., anti-PDl, anti-LAG3, etc.); binds to a checkpoint inhibitor; or binds to a peptide-MHC complex. For example, tumor-associated antigens that can be targeted include, but are not limited to, alphafetoprotein (AFP), α-actinin-4, A3, antigen specific for A33 antibody, ART-4, B7, Ba 733, BAGE, BrE3-antigen, CAI25, CAMEL, CAP-1, carbonic anhydrase IX, CASP-8/m, CCCL19, CCCL21, CD1, CDla, CD2, CD3, CD4, CD5, CD8, CD11A, CD14, CD15, CD 16, CD 18, CD 19, CD20, CD21, CD22, CD23, CD25, CD29, CD30, CD32b, CD33, CD37, CD38, CD40, CD40L, CD44, CD45, CD46, CD52, CD54, CD55, CD59, CD64, CD66a-e, CD67, CD70, CD70L, CD74, CD79a, CD80, CD83, CD95, CD126, CD132, CD133, CD138, CD147, CD 154, CDC27, CDK-4/m, CDKN2A, CTLA-4, CXCR4, CXCR7, CXCL12, HIFla, colon-specific antigen-p (CSAp), CEA (CEACAM5), CEACAM6, c-Met, DAM, EGFR, EGFRvIII, EGP-1 (TROP-2), EGP-2, ELF2-M, Ep-CAM, fibroblast growth factor (FGF), Fit-1, Flt-3, folate receptor, G250 antigen, GAGE, gplOO, GRO-b, HLA-DR, HM1.24, human chorionic gonadotropin (HCG) and its subunits, HER2/neu, HMGB-1, hypoxia inducible factor (H1^{F}-1), HSP70-2M, HST-2, la, IGF-1R, BFN-g, IFN-α, IFN-b, IFN-1, IL4R, IL-6R, IL-13R, IL-15R, IL-17R, IL-18R, IL-2, IL-6, IL-8, IL-12, IL-15, DL-17, IL-18, IL-23, IL-25, insulin-like growth factor-1 (IGF-1), KC4-antigen, KS-1-antigen, KS1-4, Le-Y, LDR/FUT, macrophage migration inhibitory factor (MIF), MAGE, MAGE-3, MART-1, MART-2, NY-ESO-1, TRAG-3, mCRP, MCP-1, MIP-1A, MIP-IB, MIF, MUC1, MUC2, MUC3, MUC4, MUC5ac, MUC13, MUC16, MUM-1/2, MUM-3, NCA66, NCA95, NCA90, PAM4 antigen, pancreatic cancer mucin, PD-1 receptor, placental growth factor, p53, PLAGL2, prostatic acid phosphatase, PSA, PRAME, PSMA, PIGF, ILGF, ILGF-1R, IL-6, IL-25, RS5, RANTES, T101, SAGE, SI 00, survivin, survivin-2B, TAC, TAG-72, tenascin, TRAIL receptors, TNF-α, Tn antigen, Thomson-Friedenreich antigens, tumor necrosis antigens, VEGFR, ED-B fibronectin, WT-1,17-1 A-antigen, complement factors C3, C3a, C3b, C5a, C5, an angiogenesis marker, bcl-2, bcl-6, and Kras. In some embodiments, the IL-2 polypeptides provided herein can be linked to an antigen in the extracellular matrix in a tumor. In some embodiments, the IL-2 polypeptides provided herein can be linked to an immunotherapeutic agent. In some embodiments, the IL-2 polypeptides provided herein can be linked to an immune checkpoint modulator. In some embodiments, the IL-2 polypeptides provided herein can be linked to a peptide-MHC complex.

In some embodiments, the IL-2 polypeptides provided herein can be linked to an Fc domain. In some embodiments, the Fc domain contains the heavy-chain constant region 2 (CH2) and the heavy-chain constant region 3 (CH3) of an immunoglobulin, excluding the heavy-chain and light-chain variable regions, the heavy-chain constant region 1 (CHT) and the light-chain constant region 1 (CLT) of the immunoglobulin. The Fc domain can further include a hinge region in the heavy-chain constant region. Also, the Fc domain can be extended to contain a portion or the whole of the heavy-chain constant region 1 (CHT) and/or the light-chain constant region 1 (CL1), except for the heavy-chain and light-chain variable regions, as long as it has a physiological function substantially equal to or better than the native form. Further, it can be a fragment having a deletion in a relatively long portion of the amino acid sequence of CH2 and/or CH3.

In some embodiments, the Fc domain can comprise (1) a CHT domain, a CH2 domain, a CH3 domain, and a CH4 domain, (2) a CHT domain and a CH2 domain, (3) a CHT domain and a CH3 domain, (4) a CH2 domain and a CH3 domain, or (5) a combination of one or more domains and an immunoglobulin hinge region (or a portion of the hinge region).

In some embodiments, the Fc domain can originate from humans or animals. In some embodiments, the Fc domain can be derived from IgG, IgA, IgD, IgE and IgM, combinations thereof, or hybrids thereof. In some embodiments, the Fc domain can be derived from IgG1, IgG2, IgG3, or IgG4 subclasses.

In some embodiments, the IL-2 polypeptides exist partially, predominantly, or entirely in a dimeric form. Suitable methods for determining oligomeric states of a protein are well-known in the art. For example, biophysical analysis for studying the aggregation properties of proteins include without limitations analytical ultracentrifugation (AUC) (e.g., sedimentation velocity AUC (SV-AUC)), size exclusion chromatography (SEC), SEC-MALS (SEC with combined with multi-angle light scattering), dynamic light scattering (DLS), sodium dodecyl sulfate-polyacrylamide gel electrophoresis (SDS-Page), native SDS-PAGE, field flow fractionation (FFF), electron microscopy (EM), cryogenic electron microscopy (cryo-EM) Nuclear magnetic resonance (NMR), and X-ray crystallography.

In some embodiments, the IL-2 polypeptides comprise at least one intermolecular disulfide bond that stabilizes the IL-2 polypeptide in the dimeric form. In some embodiments, the disulfide bond can be formed between two cysteine residues respectively located in the two IL-2 polypeptide monomers.

In some embodiments, the IL-2 polypeptides can be stabilized in a dimeric form by forming IL-2 polypeptide-dimerization domain conjugates. Such conjugates can be formed by fusing IL-2 polypeptide monomers to a dimerization domain, wherein each of the two dimerization domain monomers is linked to one IL-2 polypeptide monomer directly or indirectly via a linker. The dimerization domain can be a homodimer or heterodimer capable of stabilizing the IL-2 polypeptides at least partially (e.g., partially, predominantly, entirely) in a dimeric form. Such dimerization domain can include a dimeric immunoglobulin Fc region, a GST protein, and the like.

Alternatively, the IL-2 polypeptides can be stabilized in a dimeric form via an in vitro modification, for example, through crosslinking with a crosslinker. Crosslinkers are reagents having reactive ends to specific functional groups (e.g., primary amines or sulfhydryls) on proteins or other molecules. Crosslinkers can join two or more molecules by a covalent bond. Crosslinkers include but are not limited to amine-to-amine crosslinkers (e.g., disuccinimidyl suberate(DSS)), amine-to-sulfhydryl crosslinkers (e.g., N-g-maleimidobutyryl-oxysuccinimide ester (GMBS)), carboxyl-to-amine crosslinkers (e.g., dicyclo-hexylcarbodiimide (DCC)), sulfhydryl-to-carbohydrate crosslinkers (e.g., N-b-maleimidopropionic acid hydrazide (BMPH)), sulfhydryl-to-sulfhydryl crosslinkers (e.g., 1,4-bismaleimidobutane (BMB)), photoreactive crosslinkers (e.g., N-5-azido-2-nitrobenzoyloxysuccinimide (ANB-NOS)), chemoselective ligation crosslinkers (e.g., NHS-PEG4-Azide).

In some embodiments, the IL-2 polypeptides provided herein are isolated. In some embodiments, the IL-2 polypeptides provided herein are substantially pure.

### 7.3 Nucleic Acids, Vectors, Cells, and Methods of Production

Also provided herein are nucleic acids having a polynucleotide sequence that encodes an IL-2 polypeptide described above. In some embodiments, nucleic acids provided herein can encompasses a polynucleotide sequence which includes only coding sequences for the IL-2 polypeptides. In some embodiments, nucleic acids provided herein can encompasses a polynucleotide sequence which includes the coding sequences for the IL-2 polypeptides as well as additional coding and/or non-coding sequences. The nucleic acids provided herein can be in the form of RNA or in the form of DNA. DNA can be cDNA, genomic DNA, or synthetic DNA, and can be double-stranded or single-stranded. Single stranded DNA can be the coding strand or non-coding (anti-sense) strand. In some embodiments, the nucleic acids of the disclosure can be mRNA. In some embodiments, provided herein are mRNAs having a polynucleotide sequence encoding the IL-2 polypeptides provided herein.

In some embodiments, the nucleic acids provided herein have a polynucleotide sequence encoding an IL-2 polypeptide having rearranged Helixes 1-4 of wildtype IL-2. In some embodiments, the nucleic acids provided herein have a polynucleotide sequence encoding an IL-2 polypeptide that comprises, from N-terminus to C-terminus, Helix 1, Helix 3, Helix 2, and Helix 4. In some embodiments, the IL-2 polypeptide comprises, from N-terminus to C-terminus, Helix 2, Helix 3, Helix 1, and Helix 4.

In some embodiments, the nucleic acids provided herein have a polynucleotide sequence encoding a shuffled IL-2 polypeptide disclosed herein. In some embodiments, the nucleic acids provided herein have a polynucleotide sequence encoding a circularly permutated IL-2 polypeptide disclosed herein. In some embodiments, the nucleic acids provided herein have a polynucleotide sequence encoding a circularly permutated variant of a truncated IL-2. In some embodiments, the nucleic acids provided herein have a polynucleotide sequence encoding a circularly permutated variant of a shuffled and truncated IL-2.

In some embodiments, the nucleic acids provided herein have a polynucleotide sequence encoding an IL-2 polypeptide comprising, from N-terminus to C-terminus, (i) H1, H3, H2 and H4, or (ii) H2, H3, H1, and H4; or a circularly permutated variant thereof; wherein H1, H2, H3, and H4 corresponds to the Helix 1, Helix 2, Helix 3, and Helix 4 of wildtype IL-2, or a variant thereof. In some embodiments, H1, H2, H3, and H4 each have the amino acid sequences of SEQ ID NOs:2, 5, 7, and 10, respectively, or a variant thereof having up to five amino acid mutations.

In some embodiments, the nucleic acids provided herein have a polynucleotide sequence encoding an IL-2 polypeptide designated as 011, 012, 013, 021, 022, 023, 031, 032, 033, 041, 042, 043, 051, 052, 053, 061, 062, 063, 071, 072, 073, 081, 082, or 083. In some embodiments, the IL-2 polypeptide has the amino acid sequence selected from the group consisting of SEQ ID NOs:20-43.

The present disclosure also provides variants of the nucleic acids described herein, wherein the variants encode, for example, fragments, analogs, and/or derivatives of an IL-2 polypeptide disclosed herein. In some embodiments, the present disclosure provides a nucleic acid having a polynucleotide sequence at least about 80% identical, at least about 85% identical, at least about 90% identical, at least about 95% identical, at least about 96% identical, at least about 97% identical, at least about 98% identical, or at least about 99% identical to a polynucleotide sequence encoding an IL-2 polypeptide described herein.

As used herein, the phrase “a nucleic acid having a polynucleotide sequence at least about 95% identical to a polynucleotide sequence” means that the nucleotide sequence of the polynucleotide sequence is identical to a reference sequence except that the polynucleotide sequence can include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence can be deleted or substituted with another nucleotide, or a number of nucleotides up to 5% of the total nucleotides in the reference sequence can be inserted into the reference sequence. These mutations of the reference sequence can occur at the 5′ or 3′ terminal positions of the reference nucleotide sequence or anywhere between those terminal positions, interspersed either individually among nucleotides in the reference sequence or in one or more contiguous groups within the reference sequence.

In some embodiments, the nucleic acids provided herein have a polynucleotide sequence encoding an IL-2 polypeptide having at least 85%, at least 90%, at least 95%, at least 98%, or 100% sequence identity to an amino acid sequence selected from the group consisting of SEQ ID NOs:20-43. In some embodiments, the polynucleotide sequences provided herein encode an IL-2 polypeptide having the amino acid sequence of SEQ ID NO:20. In some embodiments, the polynucleotide sequences provided herein encode an IL-2 polypeptide having the amino acid sequence of SEQ ID NO:21. In some embodiments, the polynucleotide sequences provided herein encode an IL-2 polypeptide having the amino acid sequence of SEQ ID NO:22. In some embodiments, the polynucleotide sequences provided herein encode an IL-2 polypeptide having the amino acid sequence of SEQ ID NO:23. In some embodiments, the polynucleotide sequences provided herein encode an IL-2 polypeptide having the amino acid sequence of SEQ ID NO:24. In some embodiments, the polynucleotide sequences provided herein encode an IL-2 polypeptide having the amino acid sequence of SEQ ID NO:25. In some embodiments, the polynucleotide sequences provided herein encode an IL-2 polypeptide having the amino acid sequence of SEQ ID NO:26. In some embodiments, the polynucleotide sequences provided herein encode an IL-2 polypeptide having the amino acid sequence of SEQ ID NO:27. In some embodiments, the polynucleotide sequences provided herein encode an IL-2 polypeptide having the amino acid sequence of SEQ ID NO:28. In some embodiments, the polynucleotide sequences provided herein encode an IL-2 polypeptide having the amino acid sequence of SEQ ID NO:29. In some embodiments, the polynucleotide sequences provided herein encode an IL-2 polypeptide having the amino acid sequence of SEQ ID NO:30. In some embodiments, the polynucleotide sequences provided herein encode an IL-2 polypeptide having the amino acid sequence of SEQ ID NO:31. In some embodiments, the polynucleotide sequences provided herein encode an IL-2 polypeptide having the amino acid sequence of SEQ ID NO:32. In some embodiments, the polynucleotide sequences provided herein encode an IL-2 polypeptide having the amino acid sequence of SEQ ID NO:33. In some embodiments, the polynucleotide sequences provided herein encode an IL-2 polypeptide having the amino acid sequence of SEQ ID NO:34. In some embodiments, the polynucleotide sequences provided herein encode an IL-2 polypeptide having the amino acid sequence of SEQ ID NO:35. In some embodiments, the polynucleotide sequences provided herein encode an IL-2 polypeptide having the amino acid sequence of SEQ ID NO:36. In some embodiments, the polynucleotide sequences provided herein encode an IL-2 polypeptide having the amino acid sequence of SEQ ID NO:37. In some embodiments, the polynucleotide sequences provided herein encode an IL-2 polypeptide having the amino acid sequence of SEQ ID NO:38. In some embodiments, the polynucleotide sequences provided herein encode an IL-2 polypeptide having the amino acid sequence of SEQ ID NO:39. In some embodiments, the polynucleotide sequences provided herein encode an IL-2 polypeptide having the amino acid sequence of SEQ ID NO:40. In some embodiments, the polynucleotide sequences provided herein encode an IL-2 polypeptide having the amino acid sequence of SEQ ID NO:41. In some embodiments, the polynucleotide sequences provided herein encode an IL-2 polypeptide having the amino acid sequence of SEQ ID NO:42. In some embodiments, the polynucleotide sequences provided herein encode an IL-2 polypeptide having the amino acid sequence of SEQ ID NO:43.

The nucleic acid variants provided herein can contain alterations in the coding regions, non-coding regions, or both. In some embodiments, nucleic acid variants provided herein can contain alterations which produce silent substitutions, additions, or deletions, but do not alter the properties or activities of the encoded polypeptide. In some embodiments, nucleic acid variants comprising silent substitutions that result in no change to the amino acid sequence of the polypeptide (due to the degeneracy of the genetic code). Nucleic acid variants can be produced for a variety of reasons, for example, to optimize codon expression for a particular host (e.g., change codons in the human mRNA to those preferred by a bacterial host such as E. coli). In some embodiments, a nucleic acid variant comprises at least one silent mutation in a non-coding or a coding region of the sequence.

In some embodiments, a nucleic acid variant is produced to modulate or alter expression (or expression levels) of the encoded polypeptide. In some embodiments, a nucleic acid variant is produced to increase expression of the encoded polypeptide. In some embodiments, a nucleic acid variant is produced to decrease expression of the encoded polypeptide.

In some embodiments, the nucleic acids provided herein can have a codon-optimized sequence. For example, the polynucleotide sequence encoding the IL-2 polypeptide can be codon-optimized for expression in a eukaryote or eukaryotic cell. In some embodiments, the codon-optimized variant is codon-optimized for operability in a eukaryotic cell or organism, e.g., a yeast cell, or a mammalian cell or organism, including a mouse cell, a rat cell, and a human cell or non-human eukaryote organism.

Generally, codon optimization refers to a process of modifying a nucleic acid sequence to enhance expression in the host cells by substituting at least one codon of the native sequence with codons that are more frequently or most frequently used in the genes of that host cell while maintaining the native amino acid sequence. Various species exhibit a particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.oijp/codon/, and these tables can be adapted in a number of ways. See Nakamura et al., Nucl. Acids Res. 28:292 (2000). Computer algorithms for codon optimizing a particular sequence for expression in a particular host cell are also available, such as Gene Forge (Aptagen; Jacobus, Pa.). In some embodiments, one or more codons (e.g., 1, 2, 3, 4, 5, 10, 15, 20, 25, 50, or more, or all codons) in a sequence encoding a DNA/RNA-targeting IL-2 polypeptide corresponds to the most frequently used codon for a particular amino acid. As to codon usage in yeast, reference is made to the online Yeast Genome database available at http://www.yeastgenome.org/community/codonusage.shtml, or Codon selection in yeast, Bennetzen and Hall, J Biol Chem. 1982 Mar. 25; 257(6):3026-31.

In some embodiments, a polynucleotide sequence comprises the coding sequence for a polypeptide (e.g., an IL-2 polypeptide) fused in the same reading frame to a polynucleotide sequence which aids in expression and secretion of a polypeptide from a host cell (e.g., a leader sequence which functions as a secretory sequence for controlling transport of a polypeptide). The polypeptide can have the leader sequence cleaved by the host cell to form a “mature” form of the polypeptide.

In some embodiments, a polynucleotide sequence comprises the coding sequence for a polypeptide (e.g., an IL-2 polypeptide) fused in the same reading frame to a marker or tag sequence. For example, in some embodiments, a marker sequence is a hexa-histidine tag (HIS-tag) that allows for efficient purification of the polypeptide fused to the marker. In some embodiments, a marker sequence is a hemagglutinin (HA) tag derived from the influenza hemagglutinin protein when a mammalian host (e.g., COS-7 cells) is used. In some embodiments, the marker sequence is a FLAG™ tag. In some embodiments, a marker can be used in conjunction with other markers or tags.

In some embodiments, a polynucleotide is isolated. In some embodiments, a polynucleotide is substantially pure.

Nucleic acids provided herein can be prepared, manipulated, and/or expressed using any of the well-established techniques known and available in the art. Many vectors can be used.

Vectors and cells comprising the nucleic acids provided herein are also provided herein. In some embodiments, provided herein are vectors comprising a nucleic acid provided herein. The vectors can be expression vectors. In some embodiments, vectors provided herein comprise a polynucleotide sequence encoding an IL-2 polypeptide disclosed herein.

Examples of vectors are plasmid, autonomously replicating sequences, and transposable elements. Exemplary transposon systems such as Sleeping Beauty and PiggyBac can be used, which can be stably integrated into the genome (e.g., Ivics et al., Cell, 91 (4): 501-510 (1997); Cadiñanos et al., (2007) Nucleic Acids Research. 35 (12): e87). Additional exemplary vectors include, without limitation, plasmids, phagemids, cosmids, artificial chromosomes such as yeast artificial chromosome (YAC), bacterial artificial chromosome (BAC), or P1-derived artificial chromosome (PAC), bacteriophages such as lambda phage or M13 phage, and animal viruses. Examples of categories of animal viruses useful as vectors include, without limitation, retrovirus (including lentivirus), adenovirus, adeno-associated virus, herpesvirus (e.g., herpes simplex virus), poxvirus, baculovirus, papillomavirus, and papovavirus (e.g., SV40). Examples of expression vectors are pClneo vectors (Promega) for expression in mammalian cells; pLenti4/V5-DEST™ pLenti6/V5-DEST™, and pLenti6.2/V5-GW/lacZ (Invitrogen) for lentivirus-mediated gene transfer and expression in mammalian cells.

In some embodiments, the vector is an episomal vector or a vector that is maintained extrachromosomally. As used herein, the term “episomal” refers to a vector that is able to replicate without integration into host's chromosomal DNA and without gradual loss from a dividing host cell also meaning that said vector replicates extrachromosomally or episomally. The vector is engineered to harbor the sequence coding for the origin of DNA replication or “ori” from a lymphotrophic herpes virus or a gamma herpesvirus, an adenovirus, SV40, a bovine papilloma virus, or a yeast, specifically a replication origin of a lymphotrophic herpes virus or a gamma herpesvirus corresponding to oriP of EBV. In some embodiments, the lymphotrophic herpes virus can be Epstein Barr virus (EBV), Kaposi's sarcoma herpes virus (KSHV), Herpes virus saimiri (HS), or Marek's disease virus (MDV). Epstein Barr virus (EBV) and Kaposi's sarcoma herpes virus (KSHV) are also examples of a gamma herpesvirus. Typically, the host cell comprises the viral replication transactivator protein that activates the replication.

Useful expression vectors for bacterial hosts include known bacterial plasmids, such as plasmids from E. coli, including pCR1, pBR322, pMB9 and their derivatives, and wider host range plasmids, such as M13 and other filamentous single-stranded DNA phages.

In some embodiments, provided herein are recombinant expression vectors, which can be used to amplify and express a polynucleotide encoding an IL-2 polypeptide described herein. For example, a recombinant expression vector can be a replicable DNA construct that includes synthetic or cDNA-derived DNA fragments encoding an IL-2 polypeptide disclosed herein, operatively linked to suitable transcriptional and/or translational regulatory elements derived from mammalian, microbial, viral or insect genes. In some embodiments, a viral vector is used. DNA regions are “operatively linked” when they are functionally related to each other. For example, a promoter is operatively linked to a coding sequence if it controls the transcription of the sequence; or a ribosome binding site is operatively linked to a coding sequence if it is positioned to permit translation. In some embodiments, structural elements intended for use in certain expression systems include a leader sequence enabling extracellular secretion of translated protein by a host cell. In some embodiments, in situations where recombinant protein is expressed without a leader or transport sequence, a polypeptide can include an N-terminal methionine residue.

“Expression control sequences,” “control elements,” or “regulatory sequences” present in an expression vector are those non-translated regions of the vector-origin of replication, selection cassettes, promoters, enhancers, translation initiation signals (Shine Dalgarno sequence or Kozak sequence) introns, a polyadenylation sequence, 5′ and 3′ untranslated regions-which interact with host cellular proteins to carry out transcription and translation. Such elements can vary in their strength and specificity. Depending on the vector system and host utilized, any number of suitable transcription and translation elements, including ubiquitous promoters and inducible promoters can be used.

Illustrative ubiquitous expression control sequences that can be used in present disclosure include, but are not limited to, a cytomegalovirus (CMV) immediate early promoter, a viral simian virus 40 (SV40) promoter (e.g., early or late), a Moloney murine leukemia virus (MoMLV) LTR promoter, a Rous sarcoma virus (RSV) LTR, a herpes simplex virus (HSV) (thymidine kinase) promoter, H5, P7.5, and P11 promoters from vaccinia virus, an elongation factor 1-alpha (EFla) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), 0-kinesin (O-KIN), the human ROSA 26 locus (Irions et al., Nature Biotechnology 25, 1477-1482 (2007)), a Ubiquitin C promoter (UBC), a phosphoglycerate kinase-1 (PGK) promoter, a cytomegalovirus enhancer/chicken (3-actin (CAG) promoter, and a β-actin promoter.

Illustrative examples of inducible promoters/systems include, but are not limited to, steroid-inducible promoters such as promoters for genes encoding glucocorticoid or estrogen receptors (inducible by treatment with the corresponding hormone), metallothionine promoter (inducible by treatment with various heavy metals), MX-1 promoter (inducible by interferon), the “GeneSwitch” mifepristone-regulatable system (Sirin et al., 2003, Gene, 323:67), the cumate inducible gene switch (WO 2002/088346), tetracycline-dependent regulatory systems, etc. The IL-2 polypeptides described herein can be produced by any method known in the art, including chemical synthesis and recombinant expression techniques. The practice of the invention employs, unless otherwise indicated, conventional techniques in molecular biology, microbiology, genetic analysis, recombinant DNA, organic chemistry, biochemistry, PCR, oligonucleotide synthesis and modification, nucleic acid hybridization, and related fields within the skill of the art. These techniques are described in the references cited herein and are fully explained in the literature. See, e.g, Maniatis et al. (1982) MOLECULAR CLONING: A LABORATORY MANUAL, Cold Spring Harbor Laboratory Press; Sambrook et al. (1989), MOLECULAR CLONING: A LABORATORY MANUAL, Second Edition, Cold Spring Harbor Laboratory Press; Sambrook et al. (2001) MOLECULAR CLONING: A LABORATORY MANUAL, Cold Spring Harbor Laboratory Press, Cold Spring Harbor, NY; Ausubel et al., CURRENT PROTOCOLS IN MOLECULAR BIOLOGY, John Wiley & Sons (1987 and annual updates); CURRENT PROTOCOLS IN IMMUNOLOGY, John Wiley & Sons (1987 and annual updates) Gait (ed.) (1984) OLIGONUCLEOTIDE SYNTHESIS: A PRACTICAL APPROACH, IRL Press; Eckstein (ed.) (1991) OLIGONUCLEOTIDES AND ANALOGUES: A PRACTICAL APPROACH, IRL Press; Birren et al. (eds.) (1999) GENOME ANALYSIS: A LABORATORY MANUAL, Cold Spring Harbor Laboratory Press; Borrebaeck (ed.) (1995); each of which is incorporated herein by reference in its entirety.

The IL-2 polypeptides described herein can be produced and isolated using methods known in the art. Peptides can be synthesized, in whole or in part, using chemical methods (see, e.g., Caruthers (1980). Nucleic Acids Res. Symp. Ser. 215; Horn (1980); and Banga, A. K., THERAPEUTIC PEPTIDES AND PROTEINS, FORMULATION, PROCESSING AND DELIVERY SYSTEMS (1995) Technomic Publishing Co., Lancaster, PA). Peptide synthesis can be performed using various solid phase techniques (see, e.g., Roberge, Science 269:202 (1995); Merrifield, Methods. Enzymol. 289:3 (1997)) and automated synthesis can be achieved, e.g., using the ABI 431A Peptide Synthesizer (Perkin Elmer) in accordance with the manufacturer's instructions. Peptides can also be synthesized using combinatorial methodologies. Synthetic residues and polypeptides can be synthesized using a variety of procedures and methodologies known in the art (see, e.g., ORGANIC SYNTHESES COLLECTIVE VOLUMES, Gilman, et al. (Eds) John Wiley & Sons, Inc., NY).

Modified peptides can be produced by chemical modification methods (see, for example, Belousov, Nucleic Acids Res. 25:3440 (1997); Frenkel, Free Radic. Biol. Med. 19:373 (1995); and Blommers, Biochemistry 33:7886 (1994)). Peptide sequence variations, derivatives, substitutions and modifications can also be made using methods such as oligonucleotide-mediated (site-directed) mutagenesis, alanine scanning, and PCR based mutagenesis. Site-directed mutagenesis (Carter et al., Nucl. Acids Res., 13:4331 (1986); Zoller et al., Nucl. Acids Res. 10:6487 (1987)), cassette mutagenesis (Wells et al., Gene 34:315 (1985)), restriction selection mutagenesis (Wells et al., Philos. Trans. R. Soc. London SerA 317:415 (1986)) and other techniques can be performed on cloned DNA to produce invention peptide sequences, variants, fusions and chimeras, and variations, derivatives, substitutions and modifications thereof.

A wide variety of expression host/vector combinations can be employed. Some are described herein. Suitable host cells for expression include prokaryotes, yeast cells, insect cells, or higher eukaryotic cells under the control of appropriate promoters. Appropriate cloning and expression vectors for use with bacterial, fungal, yeast, and mammalian cellular hosts, as well as methods of protein production are well-known in the art.

Examples of suitable mammalian host cell lines include, but are not limited to, COS-7 (monkey kidney-derived), L-929 (murine fibroblast-derived), C127 (murine mammary tumor-derived), 3T3 (murine fibroblast-derived), CHO (Chinese hamster ovary-derived), HeLa (human cervical cancer-derived), BHK (hamster kidney fibroblast-derived), HEK-293 (human embryonic kidney-derived) cell lines and variants thereof. Mammalian expression vectors can comprise non-transcribed elements such as an origin of replication, a suitable promoter and enhancer linked to the gene to be expressed, and other 5′ or 3′ flanking non-transcribed sequences, and 5′ or 3′ non-translated sequences, such as necessary ribosome binding sites, a polyadenylation site, splice donor and acceptor sites, and transcriptional termination sequences. Expression of recombinant proteins in insect cell culture systems (e.g., baculovirus) also offers a robust method for producing correctly folded and biologically functional proteins. Baculovirus systems for production of heterologous proteins in insect cells are well-known to those of skill in the art.

### 7.4 Compositions and Kits

Provided herein are also compositions, e.g., pharmaceutical compositions, containing IL-2 polypeptides provided herein. Provided herein are also compositions, e.g., pharmaceutical compositions, containing a nucleic acid having a polynucleotide sequence encoding an IL-2 polypeptide provided herein. In some embodiments, the nucleic acid is an mRNA. In some embodiments, pharmaceutical compositions provided herein further comprise a pharmaceutically acceptable carrier. Such compositions can include one or a combination of (e.g., two or more different) IL-2 polypeptides provided herein. In some embodiments, the pharmaceutical compositions provided herein are useful in immunotherapy. In some embodiments, the pharmaceutical compositions are useful in immuno-oncology. In some embodiments, the pharmaceutical compositions are useful in inhibiting tumor growth in a subject (e.g., a human patient). In some embodiments, the pharmaceutical compositions are useful in treating cancer in a subject (e.g., a human patient).

In some embodiments, the pharmaceutical compositions provided herein comprise IL-2 polypeptides disclosed herein. The IL-2 polypeptides can be present at various concentrations. In some embodiments, the pharmaceutical compositions provided herein comprise IL-2 polypeptides provided herein at 1-1000 mg/ml. In some embodiments, the pharmaceutical compositions comprise IL-2 polypeptides provided herein at 10-500 mg/ml, 10-400 mg/ml, 10-300 mg/ml, 10-200 mg/ml, 10-100 mg/ml, 20-100 mg/ml, or 50-100 mg/ml. In some embodiments, the pharmaceutical compositions provided herein comprise IL-2 polypeptides at about 10 mg/ml, about 20 mg/ml, about 30 mg/ml, about 40 mg/ml, about 50 mg/ml, about 60 mg/ml, about 70 mg/ml, about 80 mg/ml, about 90 mg/ml, about 100 mg/ml, about 120 mg/ml, about 150 mg/ml, about 180 mg/ml, about 200 mg/ml, about 300 mg/ml, about 500 mg/ml, about 800 mg/ml, or about 1000 mg/ml.

Provided herein are also kits for preparation of pharmaceutical compositions having the IL-2 polypeptides disclosed herein. Provided herein are also kits for preparation of pharmaceutical compositions having the nucleic acids (e.g., mRNAs) having polynucleotide sequences encoding IL-2 polypeptides disclosed herein. In some embodiments, the kit comprises the IL-2 polypeptides or the nucleic acid (e.g., mRNAs) disclosed herein and a pharmaceutically acceptable carrier in one or more containers. In another embodiment, the kits can comprise IL-2 polypeptides or the nucleic acid (e.g., mRNAs) disclosed herein for administration to a subject. In specific embodiments, the kits comprise instructions regarding the preparation and/or administration of the IL-2 polypeptides or the nucleic acid (e.g., mRNAs) disclosed herein.

In some embodiments, pharmaceutical compositions comprising IL-2 polypeptides provided herein are suitable for local administration. In some embodiments, pharmaceutical compositions comprising nucleic acids (e.g., mRNAs) having polynucleotide sequences encoding the IL-2 polypeptides provided herein are suitable for local administration. In some embodiments, local administration comprises intratumoral injection, peritumoral injection, juxtatumoral injection, intralesional injection and/or injection into a tumor draining lymph node, or essentially any tumor-targeted injection where the antitumor agent is expected to leak into primary lymph nodes adjacent to targeted solid tumor.

Pharmaceutical compositions of the IL-2 polypeptides or nucleic acids (e.g., mRNAs) provided herein can be prepared by mixing the IL-2 polypeptides or nucleic acids (e.g., mRNAs) having the desired degree of purity with optional physiologically acceptable carriers, excipients or stabilizers (REMINGTON'S PHARMACEUTICAL SCIENCES, 16th edition, Osol, A. Ed. (1980)), in the form of lyophilized formulations or aqueous solutions. Pharmaceutically acceptable carriers that can be used in compositions provided herein include any and all solvents, dispersion media, coatings, antibacterial and antifungal agents, isotonic and absorption delaying agents, and the like that are physiologically compatible. In some embodiments, the carrier is suitable for intravenous, intramuscular, subcutaneous, parenteral, spinal or epidermal administration (e.g., by injection or infusion). Depending on the route of administration, the active ingredient (i.e., IL-2 polypeptides provided herein), can be coated in a material to protect the active ingredient from the action of acids and other natural conditions that can inactivate the active ingredient.

Provided herein are also pharmaceutical compositions that improve the stability of the IL-2 polypeptides or nucleic acids (e.g., mRNAs) to allow for their long-term storage. In some embodiments, the pharmaceutical composition disclosed herein comprises: (a) IL-2 polypeptides disclosed herein; (b) a buffering agent; (c) a stabilizing agent; (d) a salt; (e) a bulking agent; and/or (f) a surfactant. In some embodiments, the pharmaceutical composition or formulation is stable for at least 1 month, at least 2 months, at least 3 months, at least 6 months, at least 1 year, at least 2 years, at least 3 years, at least 5 years or more. In some embodiments, the pharmaceutical composition or formulation is stable when stored at 4° C., 25° C., or 40° C. In some embodiments, the pharmaceutical composition disclosed herein comprises: (a) nucleic acids (e.g., mRNAs) having polynucleotide sequences encoding the IL-2 polypeptides provided herein; (b) a buffering agent; (c) a stabilizing agent; (d) a salt; (e) a bulking agent; and/or (f) a surfactant. In some embodiments, the pharmaceutical composition or formulation is stable for at least 1 month, at least 2 months, at least 3 months, at least 6 months, at least 1 year, at least 2 years, at least 3 years, at least 5 years or more. In some embodiments, the pharmaceutical composition or formulation is stable when stored at 4° C., 25° C., or 40° C.

The pharmaceutical compositions disclosed herein can further comprise one or more of a buffer system, a preservative, a tonicity agent, a chelating agent, a stabilizer and/or a surfactant, as well as various combinations thereof. The use of preservatives, isotonic agents, chelating agents, stabilizers and surfactants in pharmaceutical compositions is well-known to the skilled person. Reference can be made to Remington: The Science and Practice of Pharmacy, 19^{th} edition, 1995. Pharmaceutical compositions disclosed herein can also include a pharmaceutically acceptable antioxidant. These compositions can also contain adjuvants such as preservatives, wetting agents, emulsifying agents and dispersing agents.

In some embodiments, the pharmaceutical composition is an aqueous formulation. Such a formulation is typically a solution or a suspension, but can also include colloids, dispersions, emulsions, and multi-phase materials. The term “aqueous formulation” is defined as a formulation comprising at least 50% w/w water. Likewise, the term “aqueous solution” is defined as a solution comprising at least 50% w/w water, and the term “aqueous suspension” is defined as a suspension comprising at least 50% w/w water.

In some embodiments, the pharmaceutical compositions disclosed herein are freeze-dried, to which the physician or the patient adds solvents and/or diluents prior to use.

Examples of suitable aqueous and nonaqueous carriers that can be employed in the pharmaceutical compositions or formulations described herein include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active ingredient, use thereof in the pharmaceutical compositions described herein is contemplated. Acceptable carriers, excipients, or stabilizers are nontoxic to recipients at the dosages and concentrations employed and include preservatives (e.g., octadecyl dimethyl benzyl ammonium chloride; hexamethonium chloride; benzalkonium chloride, benzethonium chloride; phenol, butyl or benzyl alcohol; alkyl parabens such as methyl or propylparaben; catechol; resorcinol; cyclohexanol; 3-pentanol; and m-cresol); low molecular weight (less than about 10 residues) proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN, PLEIRONIC, or polyethylene glycol (PEG).

Pharmaceutical compositions or formulations typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, the compositions can include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

In some embodiments, provided herein are pharmaceutical compositions containing a nucleic acid (e.g., mRNA) having a polynucleotide sequence encoding an IL-2 polypeptide provided herein. In some embodiments, the nucleic acid (e.g., mRNA) is encapsulated in liposomes.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated herein. In the case of sterile powders for the preparation of sterile injectable solutions, some methods of preparation are vacuum drying and freeze-drying (lyophilization) that yield a powder of the active ingredient plus any additional desired ingredient from a previously sterile-filtered solution thereof.

The pharmaceutical compositions disclosed herein can be prepared with carriers that protect the active ingredient against rapid release, such as a controlled release formulation, including implants, transdermal patches, and microencapsulated delivery systems. Many methods for the preparation of such formulations are patented or generally known to those skilled in the art. See. e.g., Sustained and Controlled Release Drug Delivery Systems, J. R. Robinson, ed., Marcel Dekker, Inc., New York, 1978. Biodegradable, biocompatible polymers can be used, such as ethylene vinyl acetate, polyanhydrides, polyglycolic acid, collagen, polyorthoesters, and poly lactic acid. Examples of sustained-release preparations include semipermeable matrices of solid hydrophobic polymers containing the IL-2 polypeptides, which matrices are in the form of shaped articles, e.g., films, or microcapsule. Examples of sustained-releasable matrices include polyesters, hydrogels (for example, poly(2-hydroxyethyl-methacrylate), or poly(vinylalcohol)), polylactides (U.S. Pat. No. 3,773,919), copolymers of L-glutamic acid and gamma-ethyl-L-glutamate, non-degradable ethylene-vinyl acetate, degradable lactic acid-glycolic acid copolymers such as the LUPRON DEPOT (injectable microspheres composed of lactic acid-glycolic acid copolymer and leuprolide acetate), and poly-d(-)-3-hydroxybutyric acid (PHB). While polymers such as ethylene-vinyl acetate and lactic acid-glycolic acid enable release of molecules for over 100 days, certain hydrogels release proteins for shorter time periods.

The amount of active ingredient which can be combined with a carrier material to produce a single dosage form will vary depending upon the subject being treated and the particular mode of administration. The amount of active ingredient which can be combined with a carrier material to produce a single dosage form will generally be that amount of the composition which produces a therapeutic effect. Generally, out of one hundred percent, this amount will range from about 0.01 percent to about ninety-nine percent of active ingredient, preferably from about 0.1 percent to about 70 percent, most preferably from about 1 percent to about 30 percent of active ingredient in combination with a pharmaceutically acceptable carrier.

The compositions provided herein can also contain more than one active ingredient as necessary for the particular indication being treated, preferably those with complementary activities that do not adversely affect each other. For instance, the formulation can further comprise another IL-2 polypeptide or IL-2 polypeptide-encoding nucleic acid (e.g., mRNA), cytotoxic agent, or a chemotherapeutic agent. Such molecules are suitably present in combination in amounts that are effective for the purpose intended.

In some embodiments, the pharmaceutical compositions provided herein can include at least an additional therapeutic agent. For example, the pharmaceutical compositions can further include an anti-cancer or anti-tumor agent, including chemotherapeutic agents and immunotherapeutic agents.

A “chemotherapeutic agent” is a chemical compound useful in the treatment of cancer. Examples of chemotherapeutic agents include alkylating agents such as thiotepa and cyclophosphamide (CYTOXAN™); alkyl sulfonates such as busulfan, improsulfan and piposulfan; aziridines such as benzodopa, carboquone, methyldopa, and uredopa; ethylenimines and methylamelamines including altretamine, triethylenemelamine, trietylenephosphoramide, triethylenethiophosphaoramide and trimethylolomelamine; acetogenins (especially bullatacin and bullatacinone); a camptothecin (including the synthetic analogue topotecan); bryostatin; cally statin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189, and CBI-TMI); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as the enediyne antibiotics (e.g., calicheamicin, see, e.g., Agnew Chem. Inti. Ed. Engl. 33:183-186 (1994); dynemicin, including dynemicin A; an esperamicin; as well as neocarzinostatin chromophore and related chromoprotein enediyne antibiotic chromomophores), aclacinomysins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; podophyllinic acid; 2-ethylhydrazide; procarbazine; PSK®; razoxane; rhizoxin; sizofuran; spirogermanium; tenuazonic acid; triaziquone; 2,2,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel (TAXOL®, Bristol-Myers Squibb Oncology, Princeton, N.J.) and doxetaxel (TAXOTERE®, Rhone-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluorom ethyl ornithine (DMFO); retinoic acid; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY 117018, onapristone, and toremifene (Fareston); and anti-androgens such as flutamide, nilutamide, bicalutamide, leuprolide, xeloda, gemcitabine, KRAS mutation covalent inhibitors, and goserelin; and pharmaceutically acceptable salts, acids or derivatives of any of the above. Additional examples include irinotecan, oxaliplatinum, and other standard colon cancer regimens.

An “immunotherapeutic agent” is a biological agent useful in the treatment of cancer. Examples of immunotherapeutic agents include atezolizumab, avelumab, blinatumomab, daratumumab, cemiplimab, durvalumab, elotuzumab, laherparepvec, ipilimumab, nivolumab, obinutuzumab, ofatumumab, pembrolizumab, cetuximab, talimogene, and the like.

Alternatively, the immunotherapeutic agent can be an immune checkpoint modulator, such as an antibody specific for CTLA-4, PD-1, PD-L1, PD-L2, killer immunoglobulin receptor (KIR), LAG3, B7-H3, B7-H4, TIM3, A2aR, CD40L, CD27, OX40, 4-IBB, TCR, BTLA, ICOS, CD28, CD80, CD86, ICOS-L, B7-H4, HVEM, 4-1BBL, OX40L, CD70, CD40, GALS or the like.

In some embodiments, IDO inhibitors can also be combined with the above treatments to further enhance the anti-tumor activity of the treatment of the IL-2 polypeptides or IL-2 polypeptides-encoding nucleic acids (e.g., mRNAs).

Provided herein are also kits comprising one or more components, such as the above-described IL-2 polypeptides, the nucleic acids encoding the IL-2 polypeptides, the vector including the polynucleotide, the host cell, or a combination thereof. The kits provided herein can be provided at any suitable temperature. For example, for storage of kits containing protein components or complexes thereof in a liquid, it is preferred that they are provided and maintained below 0° C., preferably at or below −20° C., or otherwise in a frozen state.

A kit can contain the components in an amount sufficient for single use. In some applications, one or more components can be provided in pre-measured single-use amounts in individual, typically disposable, tubes, or equivalent containers. The amount of a component supplied in the kit can be any appropriate amount and can depend on the target market to which the product is directed. The container(s) in which the components are supplied can be any conventional container that is capable of holding the supplied form, for instance, microfuge tubes, microtiter plates, ampoules, bottles, or integral testing devices, such as fluidic devices, cartridges, lateral flow, or other similar devices.

The kits can also include packaging materials for holding the container or combination of containers. Typical packaging materials for such kits and systems include solid matrices (e.g., glass, plastic, paper, foil, micro-particles and the like) that hold the components in any of a variety of configurations (e.g., in a vial, microtiter plate well, microarray, and the like). The kits can further include instructions recorded in a tangible form for the use of the components.

In some embodiments, provided herein are pharmaceutical kits of parts comprising an IL-2 polypeptide described herein and an anti-cancer or anti-tumor agent, such as an immune checkpoint modulator (e.g., an anti-CTLA-4 and/or anti-PD-1 antibody). The kit can further comprise instructions for use in the treatment of a tumor or cancer. In some embodiments, the IL-2 polypeptide and/or the anti-cancer or anti-tumor agent can be co-packaged in a unit dosage form.

In some embodiments, provided herein are pharmaceutical kits of parts comprising an IL-2 polypeptide-encoding nucleic acid (e.g., mRNA) described herein and an anti-cancer or anti-tumor agent, such as an immune checkpoint modulator (e.g., an anti-CTLA-4 and/or anti-PD-1 antibody). The kit can further comprise instructions for use in the treatment of a tumor or cancer. In some embodiments, the IL-2 polypeptide-encoding nucleic acid (e.g., mRNA) and/or the anti-cancer or anti-tumor agent can be co-packaged in a unit dosage form.

### 7.5 Methods of Uses

The IL-2 polypeptides, IL-2 polypeptide-encoding nucleic acids (e.g., mRNAs), compositions and methods described herein have numerous in vitro and in vivo utilities involving, for example, activation of immune effector cells and/or enhancement of immune response. In some embodiments, the IL-2 polypeptides or IL-2 polypeptide-encoding nucleic acids (e.g., mRNAs) described herein can be administered to cells in culture, in vitro or ex vivo, or to human subjects, e.g., in vivo, to enhance immunity in a variety of diseases. Accordingly, provided herein are methods of modifying an immune response in a subject comprising administering to the subject an IL-2 polypeptide described herein such that the immune response in the subject is modified. Provided herein are also methods of modifying an immune response in a subject comprising administering to the subject an IL-2 polypeptide-encoding nucleic acid (e.g., mRNA) described herein such that the immune response in the subject is modified. In some embodiments, the response is enhanced, stimulated or upregulated.

In some embodiments, provided herein are methods of inducing or stimulating immune effector cell activation comprising contacting an immune effector cell with an effective amount of an IL-2 polypeptide or an IL-2 polypeptide-encoding nucleic acid (e.g., mRNA) described herein under conditions to activate the immune effector cell. In some embodiments, provided herein are methods of inducing or stimulating immune cell proliferation comprising contacting an immune cell with an effective amount of an IL-2 polypeptide or an IL-2 polypeptide-encoding nucleic acid (e.g., mRNA) described herein under conditions to induce or stimulate the proliferation of the immune effector cell. Immune effector cells include T cells, B cell, natural killer (NK) cells, NKT cells, macrophages, granulocytes, neutrophils, eosinophils, mast cells, and basophils. In some embodiments, the immune effector cell is a T cell, an NK cell, an NKT cell, a myeloid cell, a macrophage, a neutrophil, or a granulocyte. In some embodiments, the immune effector cell is a T cell. In some embodiments, the immune effector cell is a NK cell. In some embodiments, the immune effector cell is a macrophage. In some embodiments, the immune effect cells can be, for example, T cells such as CD4+ T cells, CD8+ T cells, T helper (Th) cells (e.g., Th1 cells), T cytotoxic (Tc) cells or TILs. The immune effector cells can also be NK cells, NKT cells, or myeloid cells. The immune effector cells can also be genetically engineered. For example, the immune effector cell can be T cells expressing a chimeric antigen receptor (CAR), or CAR T cells. The CAR T cells can target tumor antigens known in the art.

Any suitable indicator of activation of the immune effector cell known in the art can be used to measure the activation. Non-limiting examples of such suitable indicators include increased T cell proliferation and/or increase cytokine production in the presence of the IL-2 polypeptides or the IL-2 polypeptide-encoding nucleic acid (e.g., mRNA).

Subjects suitable for the present methods include human patients in whom enhancement of an immune response would be desirable. The methods are particularly suitable for treating human patients having a disorder that can be treated by augmenting an immune response (e.g., a T-cell mediated immune response, e.g., an antigen specific T cell response). In some embodiments, the subject can be a human or non-human mammal that exhibits one or more symptoms or indications of cancer or tumor, and/or who has been diagnosed with tumor or cancer, including a solid tumor and who needs treatment for the same. In some embodiments, the subject can have primary, established, or recurrent tumor lesions. In some embodiments, the subject can be human subjects that have and/or need treatment for a solid tumor. In some embodiments, the subject can have primary or metastatic tumors (advanced malignancies). In some embodiments, the subject can have a solid tumor that is resistant to or refractory to or is inadequately controlled by prior therapy (e.g., surgery or treatment with an anti-cancer agent such as carboplatin or docetaxel). In some embodiments, the subject can have a tumor lesion that has been treated with one or more lines of prior therapy (e.g., surgically removed), but which has subsequently recurred. In some embodiments, the subject can have a tumor or cancer who are not candidates for curative surgery or curative radiation, or for whom conventional anti-cancer therapy is inadvisable, for example, due to toxic side effects. In other embodiments, the subject can have a tumor lesion for which surgical removal is planned. In other embodiments, the subject can have a high risk of recurrence is high due to a prior history of recurrence after surgery.

As used interchangeably herein, the term “cancer,” “tumor,” and “malignancy” all relate equivalently to hyperplasia of a tissue or organ. If the tissue is a part of the lymphatic or immune system, malignant cells can include non-solid tumors of circulating cells. Malignancies of other tissues or organs can produce solid tumors. The methods described herein can be used in the treatment of lymphatic cells, circulating immune cells, and solid tumors.

In some embodiments, provided herein are methods of uses of the IL-2 polypeptides, nucleic acids encoding such IL-2 polypeptides, vectors comprising such nucleic acids, or pharmaceutical compositions having such IL-2 polypeptides or nucleic acids disclosed herein in treating cancer. In some embodiments, the methods include administering a therapeutically effective amount of the IL-2 polypeptides disclosed herein to a subject in need thereof. In some embodiments, provided herein are uses of the IL-2 polypeptides disclosed herein in the treatment of tumor or cancer. In some embodiments, provided herein are uses of the IL-2 polypeptides disclosed herein for the preparation of a medicament for the treatment of tumor or cancer.

In some embodiments, the methods include administering a therapeutically effective amount of the IL-2 polypeptides-encoding nucleic acids (e.g., mRNAs) disclosed herein to a subject in need thereof. In some embodiments, provided herein are uses of the IL-2 polypeptides-encoding nucleic acids (e.g., mRNAs) disclosed herein in the treatment of tumor or cancer. In some embodiments, provided herein are uses of the IL-2 polypeptides-encoding nucleic acids (e.g., mRNAs) disclosed herein for the preparation of a medicament for the treatment of tumor or cancer.

In some embodiments, provided herein are methods of treating tumor or cancer in a subject in need thereof, comprising administering to the subject a therapeutically effective amount of the pharmaceutical composition disclosed herein. In some embodiments, provided herein are uses of the pharmaceutical composition disclosed herein in treatment of tumor or cancer. In some embodiments, provided herein are uses of the pharmaceutical composition provided herein for the preparation of a medicament for the treatment of tumor or cancer.

Dosage regimens can be adjusted to provide the optimum desired response (e.g., a therapeutic response). For example, a single bolus can be administered, several divided doses can be administered over time, or the dose can be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active ingredient calculated to produce the desired therapeutic effect in association with the required pharmaceutical carrier. The specification for the dosage unit forms can be dictated by and directly dependent on (a) the unique characteristics of the active compound and the particular therapeutic effect to be achieved, and (b) the limitations inherent in the art of compounding such an active ingredient for the treatment of sensitivity in individuals.

For administration of the IL-2 polypeptides, the dosage ranges from about 0.0001 to 100 mg/kg, and more usually 0.01 to 5 mg/kg, of the host body weight. For example, dosages can be 0.3 mg/kg body weight, 1 mg/kg body weight, 3 mg/kg body weight, 5 mg/kg body weight or 10 mg/kg body weight or within the range of 1-10 mg/kg. An exemplary treatment regime entails administration daily, twice per week, once per week, once every two weeks, once every three weeks, once every four weeks, once a month, once every 3 months or once every three to 6 months. For example, dosage regimens for an IL-2 polypeptide described herein can be 0.2-10 mg/kg body weight via intravenous administration.

Alternatively, the IL-2 polypeptide or the IL-2 polypeptides-encoding nucleic acids (e.g., mRNAs) can be administered as a sustained release formulation, in which case less frequent administration is required. Dosage and frequency vary depending on the half-life of the IL-2 polypeptide or the IL-2 polypeptide-encoding nucleic acid (e.g., mRNA) in the patient. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patient can be administered a prophylactic regime.

Actual dosage levels of the active ingredients (i.e., the IL-2 polypeptides or the IL-2 polypeptides-encoding nucleic acids (e.g., mRNAs) disclosed herein) in the pharmaceutical compositions described herein can be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions described herein, the route of administration, the time of administration, the rate of excretion, the duration of the treatment, other drugs, compounds and/or materials used in combination with the particular compositions employed, the age, sex, weight, condition, general health and prior medical history of the patient being treated, and like factors well known in the medical arts.

The IL-2 polypeptides, IL-2 polypeptides-encoding nucleic acids (e.g., mRNAs), or pharmaceutical compositions provided herein can be administered to a subject by any methods known in the art. As will be appreciated by the skilled artisan, the route and/or mode of administration will vary depending upon the desired results. Preferred routes of administration include intravenous, intramuscular, intradermal, intraperitoneal, subcutaneous, spinal or other parenteral routes of administration, for example by injection or infusion. Other routes of administration include, for example, pleural administration, intranodal administration, intratumoral administration, intrathecal administration, intrapleural administration, intraperitoneal administration, intracranial administration, spinal or other parenteral routes of administration, for example by injection or infusion, or direct administration to the thymus. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrastemal injection and infusion. In some embodiments, subcutaneous administration is adopted. In some embodiments, intravenous administration is adopted. In some embodiments, oral administration is adopted. In one embodiment, the IL-2 polypeptides or the IL-2 polypeptides-encoding nucleic acids (e.g., mRNAs) provided herein can be delivered regionally to a tumor using well known methods, including but not limited to, hepatic or aortic pump; limb, lung or liver perfusion; in the portal vein; through a venous shunt; in a cavity or in a vein that is nearby a tumor, and the like. In another embodiment, the IL-2 polypeptides or the IL-2 polypeptides-encoding nucleic acids (e.g., mRNAs) provided herein can be administered systemically. In a preferred embodiment, the IL-2 polypeptides or the IL-2 polypeptides-encoding nucleic acids (e.g., mRNAs) are administered regionally at the site of a tumor. The IL-2 polypeptides or the IL-2 polypeptides-encoding nucleic acids (e.g., mRNAs) can also be administered intratumorally, for example, by direct injection of the cells at the site of a tumor and/or into the tumor vasculature. For example, in the case of malignant pleural disease, mesothelioma or lung cancer, administration is preferably by intrapleural administration (see Adusumilli et al., Science Translational Medicine 6(261):261ra151 (2014)). One skilled in the art can select a suitable mode of administration based on the type of cancer and/or location of a tumor to be treated. The IL-2 polypeptides or the IL-2 polypeptides-encoding nucleic acids (e.g., mRNAs) can be introduced by injection or catheter. In one embodiment, the IL-2 polypeptides or the IL-2 polypeptides-encoding nucleic acids (e.g., mRNAs) are pleurally administered to the subject in need, for example, using an intrapleural catheter.

IL-2 polypeptides, IL-2 polypeptides-encoding nucleic acids (e.g., mRNAs), or pharmaceutical compositions provided herein can be administered with medical devices known in the art. For example, in some embodiments, a needleless hypodermic injection device can be used, such as the devices disclosed in U.S. Pat. Nos. 5,399,163; 5,383,851; 5,312,335; 5,064,413; 4,941,880; 4,790,824; or 4,596,556. Examples of well-known implants and modules for use described herein include: U.S. Pat. No. 4,487,603, which discloses an implantable micro-infusion pump for dispensing medication at a controlled rate; U.S. Pat. No. 4,486,194, which discloses a therapeutic device for administering medicaments through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. These patents are incorporated herein by reference. Many other such implants, delivery systems, and modules are known to those skilled in the art.

Cancers or tumors to be treated using the IL-2 polypeptides, the IL-2 polypeptides-encoding nucleic acids (e.g., mRNAs), or pharmaceutical compositions provided herein comprise those typically responsive to immunotherapy and those that are not typically responsive to immunotherapy. In some embodiments, the cancer has a high degree of microsatellite instability. In some embodiments, the cancer is a metastatic cancer, refractory cancer, or recurrent cancer.

Cancers that can be treated include tumors that are not vascularized or are not substantially vascularized, as well as vascularized tumors. Cancers can comprise non-solid tumors (such as hematologic tumors, e.g., leukemias and lymphomas) or comprise solid tumors. The types of cancers to be treated with the IL-2 polypeptides, the IL-2 polypeptides-encoding nucleic acids (e.g., mRNAs), and pharmaceutical compositions disclosed herein include, but are not limited to, carcinoma, blastoma and sarcoma, and certain leukemias or malignant lymphoid tumors, benign and malignant tumors and malignancies, e.g., sarcomas, carcinomas, and melanomas. Also included are adult tumors/cancers and pediatric tumors/cancers.

In some embodiments, cancers or tumors that can be treated with the IL-2 polypeptides, the IL-2 polypeptides-encoding nucleic acids (e.g., mRNAs), or pharmaceutical compositions disclosed herein are hematological cancers. In some embodiments, the hematological cancer can be leukemia, lymphoma, multiple myeloma (MM), or myelodysplastic syndrome (MDS). In some embodiments, the hematological cancer can be acute leukemia, acute myeloid leukemia (AML), B-acute lymphoid leukemia (B-ALL), T-acute lymphoid leukemia (T-ALL), B cell precursor acute lymphoblastic leukemia (BCP-ALL), blastic plasmacytoid dendritic cell neoplasm (BPDCN), acute lymphocytic leukemia, acute myeloblastic leukemia, acute promyelocytic leukemia, acute myelomonocytic leukemia, acute monocytic leukemia, acute erythroleukemia, chronic leukemia, chronic myeloid leukemia (CML), chronic myelocytic leukemia, chronic lymphocytic leukemia, chronic myelomonocytic leukemia (CMMLL), natural killer cell leukemia (NK leukemia), Hodgkin's disease, non-Hodgkin's disease, Waldenstrom's macroglobulinemia, lymphocytic lymphoma, primary CNS lymphoma, T-cell lymphoma, natural killer cell lymphoma (NK lymphoma), cutaneous T-Cell lymphoma (CTCL), or peripheral T-cell lymphoma (PTCL). In some embodiments, IL-2 polypeptides, the IL-2 polypeptides-encoding nucleic acids (e.g., mRNAs), or pharmaceutical compositions disclosed herein can be used to treat MDS. In some embodiments, IL-2 polypeptides, the IL-2 polypeptides-encoding nucleic acids (e.g., mRNAs), or pharmaceutical compositions disclosed herein can be used to treat leukemia. In some embodiments, the leukemia is AML.

In some embodiments, cancers or tumors that can be treated with the IL-2 polypeptides, the IL-2 polypeptides-encoding nucleic acids (e.g., mRNAs), or pharmaceutical compositions disclosed herein are solid tumors. In some embodiments, the cancer or tumor can be carcinomas, sarcoma, melanoma (e.g., cutaneous or intraocular malignant melanoma), glioma, glioblastoma, brain and spinal cord tumors, germ cell tumors, neuroendocrine tumors, carcinoid tumors, gastric cancer, esophageal cancer, liver cancer, lung cancer (e.g., small cell lung cancer, or non-small cell lung cancer), head and neck cancer, skin cancer, nasopharyngeal cancer, kidney cancer, colorectal cancer, breast cancer, pancreatic cancer, testicular cancer, cervical cancer, ovarian cancer, uterine cancer, prostate cancer (for example, hormone refractory prostate adenocarcinoma), bladder cancer, colon cancer, endocrine cancer, basal cell cancer, squamous cell cancer, dermatofibrosarcoma protuberans, mesothelioma, Merkel cell carcinoma, bone cancer, intestinal cancer, renal cancer (for example, clear cell carcinoma), throat cancer, rectal cancer, cancer of the anal region, brain cancer, stomach cancer, carcinoma of the fallopian tubes, carcinoma of the endometrium, carcinoma of the cervix, carcinoma of the vagina, carcinoma of the vulva, cancer of the small intestine, cancer of the thyroid gland, cancer of the parathyroid gland, cancer of the adrenal gland, sarcoma of soft tissue, cancer of the urethra, cancer of the penis, solid tumors of childhood, cancer of ureter, carcinoma of the renal pelvis, neoplasm of the central nervous system (CNS), spinal axis tumor, brain stem glioma, pituitary adenoma, Kaposi's sarcoma, epidermoid cancer, fibrosarcoma, myxosarcoma, liposarcoma, chondrosarcoma, osteogenic sarcoma, synovial sarcoma, chordoma, angiosarcoma, endotheliosarcoma, lymphangiosarcoma, lymphangioendotheliosarcoma, synovioma, Ewing's tumor, leiomyosarcoma, rhabdomyosarcoma, squamous cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, hepatoma, bile duct carcinoma, choriocarcinoma, seminoma, embryonal carcinoma, Wilm's tumor, epithelial carcinoma, glioma, astrocytoma, medulloblastoma, craniopharyngioma, ependymoma, pinealoma, hemangioblastoma, acoustic neuroma, oligodenroglioma, schwannoma, meningioma, neuroblastoma, or retinoblastoma.

In cancer treatment, eliminating cancer or tumor cells in a subject can occur, but any clinical improvement constitutes a benefit. An anti-tumor effect can be manifested by a decrease in tumor volume, a decrease in the number of tumor cells, a decrease in the number of metastases, an increase in life expectancy, or amelioration of various physiological symptoms associated with the cancerous condition. An anti-tumor effect can also be manifested by the ability of the IL-2 polypeptides, the IL-2 polypeptides-encoding nucleic acids (e.g., mRNAs), or pharmaceutical compositions provided herein in prevention of the occurrence of tumor in the first place. In some embodiments, an “anti-tumor effect” can be manifested by the reduction in cancer-induced immunosuppression. Clinical improvement comprises decreased risk or rate of progression or reduction in pathological consequences of the cancer or tumor. It is also understood that a method of treating cancer can include any effect that ameliorates a sign or symptom associated with cancer. Such signs or symptoms include, but are not limited to, reducing tumor burden, including inhibiting growth of a tumor, slowing the growth rate of a tumor, reducing the size of a tumor, reducing the number of tumors, eliminating a tumor, all of which can be measured using routine tumor imaging techniques well known in the art. Other signs or symptoms associated with cancer include, but are not limited to, fatigue, pain, weight loss, and other signs or symptoms associated with various cancers.

In some embodiments, the methods or uses provided herein can reduce tumor burden. Thus, administration of the IL-2 polypeptides, the IL-2 polypeptides-encoding nucleic acids (e.g., mRNAs), or pharmaceutical compositions disclosed herein can reduce the number of tumor cells, reduce tumor size, and/or eradicate the tumor in the subject. Methods for monitoring patient response to administration of a pharmaceutical composition disclosed herein are known in the art and can be employed in accordance with methods disclosed herein.

Treatment of a subject having cancer with an IL-2 polypeptides or the IL-2 polypeptides-encoding nucleic acids (e.g., mRNAs) provided herein, can result in, e.g., stable disease, partial response, increased overall survival, increased disease free survival, or enhanced progression free survival.

In some embodiments, the IL-2 polypeptides or the IL-2 polypeptides-encoding nucleic acids (e.g., mRNAs) provided herein are not significantly toxic. For example, IL-2 polypeptides or the IL-2 polypeptides-encoding nucleic acids (e.g., mRNAs) are not significantly toxic to an organ of a human, e.g., one or more of the liver, kidney, brain, lungs, and heart, as determined, e.g., in clinical trials. In some embodiments, the IL-2 polypeptides or the IL-2 polypeptides-encoding nucleic acids (e.g., mRNAs) do not significantly trigger an undesirable immune response, e.g., autoimmunity or inflammation. In some embodiments, treatment of a subject with IL-2 polypeptides or the IL-2 polypeptides-encoding nucleic acids (e.g., mRNAs) described herein does not cause significant inflammatory reactions, e.g., immune-mediated pneumonitis, immune-mediated colitis, immune mediated hepatitis, immune-mediated nephritis or renal dysfunction, immune-mediated hypophysitis, immune-mediated hypothyroidism and hyperthyroidism, or other immune-mediated adverse reactions.

In some embodiments, an anti-tumor effect is observed in a subject having a tumor or cancer who has been administered with IL-2 polypeptides described herein as a single therapy, namely, not in combination with another therapeutic. In some embodiments, tumor burden is reduced in a subject having a tumor or cancer who has been administered with IL-2 polypeptides described herein as a single therapy.

In some embodiments, an anti-tumor effect is observed in a subject having a tumor or cancer who has been administered with the IL-2 polypeptides-encoding nucleic acids (e.g., mRNAs) described herein as a single therapy, namely, not in combination with another therapeutic. In some embodiments, tumor burden is reduced in a subject having a tumor or cancer who has been administered with the IL-2 polypeptides-encoding nucleic acids (e.g., mRNAs) described herein as a single therapy.

In some embodiments, IL-2 polypeptides or the IL-2 polypeptides-encoding nucleic acids (e.g., mRNAs) provided herein are given to a subject as an adjunctive therapy. Treatments of subjects having cancer with an IL-2 polypeptide or the IL-2 polypeptides-encoding nucleic acids (e.g., mRNAs) provided herein can lead to prolonged survival, e.g., long-term durable response relative to the current standard of care; long term survival of at least 3 months, 6 months, 9 months, 1, 2, 3, 4, 5, 10 or more years, or recurrence-free survival of at least 3 months, 6 months, 9 months, 1, 2, 3, 4, 5, or 10 or more years. In some embodiments, treatment of a subject having cancer with an IL-2 polypeptide or the IL-2 polypeptides-encoding nucleic acids (e.g., mRNAs) provided herein prevents recurrence of cancer or delays recurrence of cancer by, e.g., 3 months, 6 months, 9 months, 1, 2, 3, 4, 5, or 10 or more years. An IL-2 polypeptide provided herein can be used as a first-, second-, or third-line treatment.

In some embodiments, treatment of a subject with IL-2 polypeptides or the IL-2 polypeptides-encoding nucleic acids (e.g., mRNAs) provided herein does not result in overstimulation of the immune system to the extent that the subject's immune system then attacks the subject itself (e.g., autoimmune response) or results in, e.g., anaphylaxis. Thus, in some embodiments, IL-2 polypeptides or the IL-2 polypeptides-encoding nucleic acids (e.g., mRNAs) provided herein do not cause anaphylaxis.

In some embodiments, IL-2 polypeptides or the IL-2 polypeptides-encoding nucleic acids (e.g., mRNAs) disclosed herein provide synergistic anti-tumor effects in combination with another cancer therapy. Pharmaceutical compositions provided herein can be administered in combination therapy, i.e., co-administered with other therapeutic agents. In some embodiments, IL-2 polypeptides or the IL-2 polypeptides-encoding nucleic acids (e.g., mRNAs) provided herein can be administered with a standard of care treatment. For example, the combination therapy can include an IL-2 polypeptide or the IL-2 polypeptides-encoding nucleic acids (e.g., mRNAs) disclosed herein combined with at least one other therapeutic agent, such as an anti-cancer or anti-tumor agent, including chemotherapeutic agents and immunotherapeutic agents, as provided above. Other combination therapies that can result in synergy with IL-2 polypeptides or the IL-2 polypeptides-encoding nucleic acids (e.g., mRNAs) through cell death are radiation, surgery, and hormone deprivation. In some embodiments, angiogenesis inhibitors can also be combined with IL-2 polypeptides or the IL-2 polypeptides-encoding nucleic acids (e.g., mRNAs) disclosed herein. IL-2 polypeptides or the IL-2 polypeptides-encoding nucleic acids (e.g., mRNAs) provided herein can also be administered as a maintenance therapy, e.g., a therapy that is intended to prevent the occurrence or recurrence of tumors. The IL-2 polypeptides or the IL-2 polypeptides-encoding nucleic acids (e.g., mRNAs) disclosed herein provided can be administered with another treatment, e.g., radiation, surgery, or chemotherapy.

As used herein, the term “co-administration” or “co-administered” refers to the administration of at least two agent(s) or therapies to a subject. In some embodiments, the co-administration of two or more agents/therapies is concurrent. In other embodiments, a first agent/therapy is administered prior to a second agent/therapy. Those of skill in the art understand that the formulations and/or routes of administration of the various agents/therapies used may vary.

For example, the IL-2 polypeptides or the IL-2 polypeptides-encoding nucleic acids (e.g., mRNAs) disclosed herein can also be combined chemotherapeutic regimes. Examples of the chemotherapeutic regimes are provided herein are known in the art. In these instances, the combination with IL-2 polypeptides or the IL-2 polypeptides-encoding nucleic acids (e.g., mRNAs) disclosed herein can reduce the dose of chemotherapeutic reagent administered (Mokyr, M. et al. (1998) Cancer Research 58: 5301-5304). An example of such a combination is an IL-2 polypeptide or an IL-2 polypeptide-encoding nucleic acid (e.g., mRNA) described herein in combination with dacarbazine for the treatment of melanoma.

In some embodiments, IL-2 polypeptides or the IL-2 polypeptides-encoding nucleic acids (e.g., mRNAs) can be combined with the antibodies used to activate host immune responsiveness. In some embodiments, such antibodies can target molecules on the surface of dendritic cells that activate DC function and antigen presentation. For example, anti-CD40 antibodies are able to substitute effectively for T cell helper activity (Ridge, J. et al. (1998) Nature 393: 474-478) and can be used in conjunction with IL-2 polypeptides. Similarly, activating antibodies to T cell costimulatory molecules such as CTLA-4 (e.g., U.S. Pat. No. 5,811,097), OX-40 (e.g., Weinberg et al. (2000) Immunol 164: 2160-2169), 4-1BB (Melero et al. (1997) Nature Medicine 3: 682-685 (1997), PD-1 (e.g., U.S. Pat. No. 8,008,449), PD-1L (e.g., U.S. Pat. Nos. 7,943,743 and 8,168,179) and ICOS (e.g., Hutloff et al. (1999) Nature 397: 262-266) can also provide for increased levels of T cell activation.

In some embodiments, IL-2 polypeptides or the IL-2 polypeptides-encoding nucleic acids (e.g., mRNAs) can be combined with adoptive cell therapies (ACT) to promote the expansion, activation, tumor infiltration and persistence of transferred cells. Such cells can include but not limited to CAR-T or CAR-NK cells, expanded tumor-infiltration lymphocytes, tumor-specific T cells, and the like.

In some embodiments, IL-2 polypeptides or the IL-2 polypeptides-encoding nucleic acids (e.g., mRNAs) disclosed herein can be used in conjunction with anti-neoplastic antibodies, such as RITUXAN (rituximab), HERCEPTIN (trastuzumab), BEXXAR (tositumomab), ZEVALIN (ibritumomab), CAMPATH (alemtuzumab), LYMPHOCIDE (pertuzumab), AVASTIN (bevacizumab), and TARCEVA (erlotinib), and the like.

In some embodiments, the present disclosure provides a method for treating a tumor, comprising co-administering an IL-2 polypeptide or an IL-2 polypeptides-encoding nucleic acid (e.g., mRNA) disclosed herein and an immune checkpoint modulator (e.g., an anti-CTLA-4 antibody) to a subject. For example, the IL-2 polypeptide or IL-2 polypeptides-encoding nucleic acid (e.g., mRNA) can be administered at a subtherapeutic dose, and the immune checkpoint modulator can be administered at a subtherapeutic dose, or both are administered at a subtherapeutic dose. In some embodiments, provided herein are methods for altering an adverse event associated with treatment of a hyperproliferative disease with an immunostimulatory agent, comprising administering an IL-2 polypeptide or an IL-2 polypeptides-encoding nucleic acid (e.g., mRNA) disclosed herein and a subtherapeutic dose of an immune checkpoint modulator to a subject.

An IL-2 polypeptide or an IL-2 polypeptides-encoding nucleic acid (e.g., mRNA) described herein can also be combined with a vaccination protocol. Dove, A. Nat. Biotechnol 17, 317 (1999). Many experimental strategies for vaccination against tumors have been devised (see Rosenberg, 2000, DEVELOPMENT OF CANCER VACCINES, ASCO Educational Book Spring: 60-62; Logothetis, C, 2000, ASCO Educational Book Spring: 300-302; Khayat, D. 2000, ASCO Educational Book Spring: 414-428; Foon, K. 2000, ASCO Educational Book Spring: 730-738; see also Restifo and Sznol, Cancer Vaccines, Ch. 61, pp. 3023-3043 in DeVita et al. (eds.), 1997, CANCER: PRINCIPLES AND PRACTICE OF ONCOLOGY, Fifth Edition). In one of these strategies, a vaccine is prepared using autologous or allogeneic tumor cells. In some embodiments, IL-2 polypeptides provided herein can boost cancer vaccine. In some embodiments, IL-2 polypeptides-encoding nucleic acids (e.g., mRNAs) provided herein can boost cancer vaccine.

All papers, publications and patents cited in this specification are herein incorporated by reference as if each individual paper, publication or patent were specifically and individually indicated to be incorporated by reference and are incorporated herein by reference to disclose and describe the methods and/or materials in connection with which the publications are cited.

However, mention of any reference, article, publication, patent, patent publication, and patent application cited herein is not, and should not be taken as an acknowledgment or any form of suggestion that they constitute valid prior art or form part of the common general knowledge in any country in the world.

Unless the context indicates otherwise, it is specifically intended that the various features described herein can be used in any combination. Unless otherwise defined, all technical and scientific terms used herein have the same meaning as commonly understood by one of ordinary skill in the art to which this disclosure belongs. The terminology used in the description of the invention herein is for the purpose of describing particular embodiments only and is not intended to be limiting of the invention.

### 7.6 Experimental

The examples provided below are for purposes of illustration only, which are not intended to be limiting unless otherwise specified. Thus, the invention should in no way be construed as being limited to the following examples, but rather, should be construed to encompass any and all variations which become evident as a result of the teaching provided herein.

### 7.6.1 Example 1: Generation of CP-trIL-2

Wild-type IL-2 has 4 α-helixes, which bind to CD122 and CD132, 2 loops that bind to CD25, and 4 linkers connecting the helixes and loops. The 133 amino acids of wt-IL-2 include: Linker 1 (aa 1-5)—Helix 1 (aa 6-29)—Linker 2 (aa 30-34)—Loop 1 (aa 35-51)—Helix 2 (aa 52-71) Linker 3 (aa 72-79)—Helix 3 (aa 80-98)—Linker 4 (aa 99-102)—Loop 2 (aa 103-112)—Helix 4 (aa 113-133). The CP-trIL-2 variants provided herein were generated as follows:

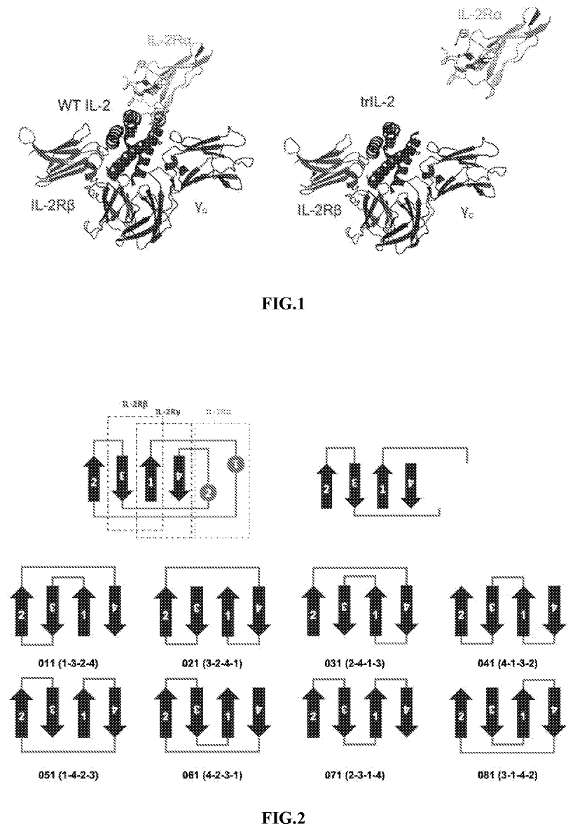

Truncation: Loop 1 and Loop 2 were removed to abolish CD25 binding (see FIG. 1)

Shuffling: The 4 helixes (Helix 1-4 as identified above) were shuffled following the sequence of 1-3-2-4 or 2-3-1-4. The linkers were designed as follows: A) Linker 1 at the N-terminus of Helix 1; B) Linker 2 at the C-terminus of Helix 1; C) Linker 3 at the C-terminus of Helix 2; D) Linker 4 at the C-terminus of Helix 3; and E) artificial linker 5 (GGGGSGGGGS) at the C-termini of Helix 4. Either Linker 2, 3, 4, or 5 would not be needed if the connecting helix is the ending helical region at C-terminus. For example, Linker 2 is not needed if Helix 1 is the ending helical region at C-terminus.

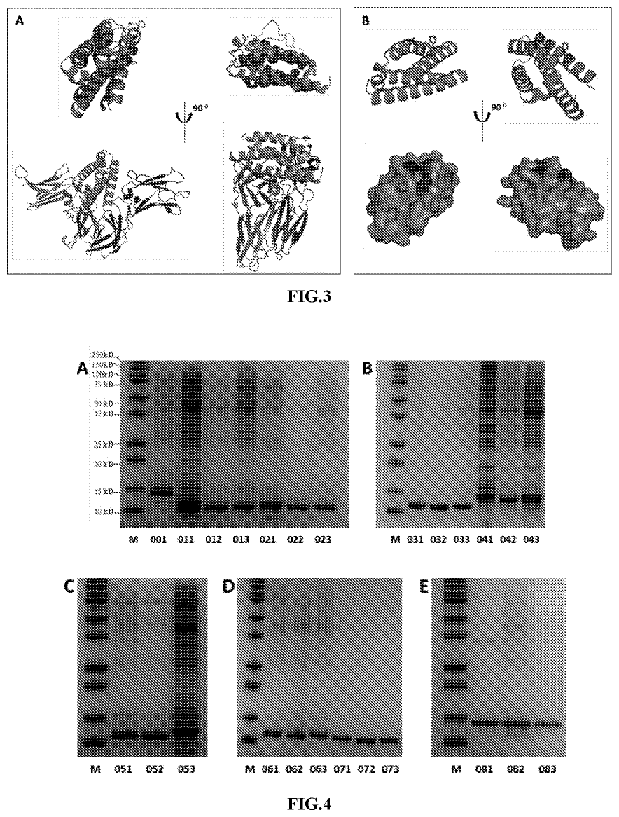

Circular permutation: The C-terminus of each helix region was selected as the cutting point for circular permutation; Linker 5 was added to the C-terminus of Helix 4 (FIG. 2).

Additional mutations: Additional mutations were introduced to CP-trIL-2 molecules to increase their expression yield and protein stability, including de-alanine-1, C58S, and C125S. Additional basic mutations, including P65D, F117K and/or F124K were also introduced to further stabilize the helixes and decrease the hydrophobicity of surface (FIG. 3). The numbering of wild-type IL-2 was used to identify the amino acid residues of the additional mutations.

### 7.6.2 Example 2: Expression and In Vitro Activity of CP-trIL-2

Expression and purification: All expression plasmids were constructed using the pET30a vector. Wild type IL-2 was named 001 and mutated to include de-alanine-1, C58S and C125S. After the steps of truncation, shuffling and circular permutation, 8 IL-2 polypeptides were generated and named 011, 021, 031, 041, 051, 061, 071 and 081, respectively (FIG. 2). P65D mutation was further introduced to the eight IL-2 polypeptides to generate 012, 022, 032, 042, 052, 062, 072 and 082, respectively. F117K and F124K were further introduced to these 8 IL-2 polypeptides above in combination with P65D to generate 013, 023, 033, 043, 053, 063, 073 and 083, respectively. The sequences of all IL-2 polypeptides are provided in Table 2.

The wildtype IL-2 (001) and all 24 variant plasmids were transformed to E. coli BL21 (DE3) and stimulated by 0.5 M IPTG to express the proteins. The bacteria were then harvested and re-suspended in the lysis buffer (50 mM Tris-HCl, 5 mM EDTA, 1 mM PMSF, pH 8.0), which was collected after the ultra-sonication. After centrifugation (12000 rpm, 30 min), the precipitates were collected (except 041, 042 and 043), washed with washing buffer (50 mM Tris-HCl, 5 mM EDTA, 300 mM NaCl, pH 8.0; 2 h, 3 times). For 041, 042 and 043, after cell lysis and centrifugation, the supernatants were collected and treated with saturated ammonium sulphate solution. The precipitates were then washed using the lysis buffer for 2 times. After washing, the inclusion bodies were de-natured in the solubilization buffer (50 mM Tris-HCl, 5 mM EDTA, 1 mM PMSF, 9 M Urea, pH 8.0) by mild shaking for 2 h. Urea concentration and pH of solubilization buffer were adjusted for certain variants. After centrifugation at 13000 rpm for 30 min, the supernatants were dropped in the 9 times volume of refolding buffer (50 mM Tris-HCl, 1 mM EDTA, 10% (w/v) sucrose, 1 mM PMSF, pH 8.0) and stood overnight at 4° C. The solutions were then centrifuged at 13000 rpm at 4° C. for 30 min. The supernatants were filtered with 0.22 μm diameter filter and stored at 4° C. For 041, 042 and 043, the supernatants were treated by the XS/Q chromatography purification, the eluted fragments were collected.

The proteins were further concentrated by centrifugation at 5000 rpm at 4° C. for 15 min as needed.

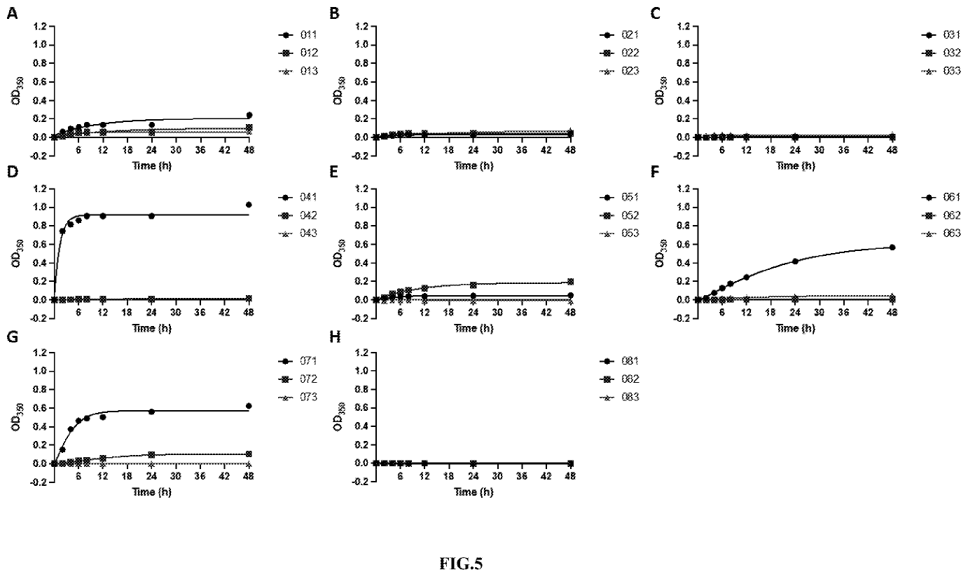

Production: The concentrations of the proteins were determined by BCA, Bradford kit, and/or SDS-PAGE. The SDS-PAGE results for all CP-trIL-2 variants were showed in FIG. 4. The expression yields were calculated and provided in Table 2.

Thermo-stability: The thermo-stability of the purified CP-trIL-2 variants at 37° C. were monitored by OD350 for 48 hours. The results are showed in FIG. 5 and summarized below.

In vitro activity: The activities of the purified CP-trIL-2 variants were confirmed by STAT5 activation assay using NK92 (expressing high-affinity receptor) and HH cell lines (expressing intermediate-affinity receptor).

NK-92 cell (Procell) expressing high-affinity (α/β/γ) receptor, and HH cells (ATCC) expressing intermediate-affinity (only β/γ) receptor were used to detect CP trIL-2 bioactivity in vitro by monitoring the IL-2-induced STAT5 phosphorylation.

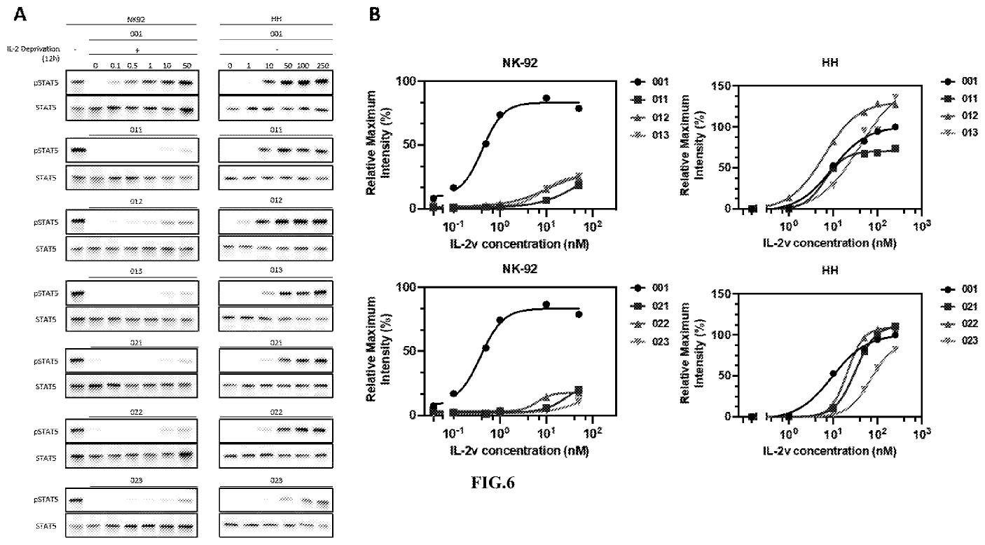

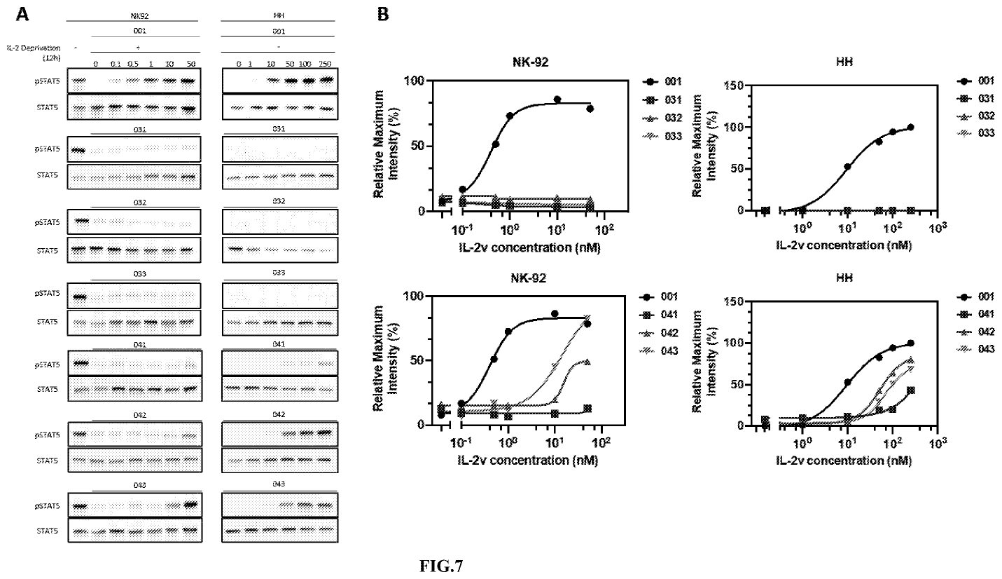

NK-92 cells were plated into 12-well plates at 500,000 cells/well in IL-2-deprived media (MyeloCult® H5100). 12 hours later, different doses of wtIL-2 or CP trIL-2 variant were added into well-plate. Similarly, HH cells were also plated into 12-well plates at 500,000 cells/well in full media (RPMI 1640 medium with 10% FBS). Different doses of wtIL-2 or CP trIL-2 were also added. The samples were incubated at 37° C., 5% CO₂ for 30 minutes. The cells were collected by centrifugation at 1500 rpm for 5 min and lysed using Laemmli Buffer. The bioactivities of the CP trIL-2 variants were indicated by STAT5 phosphorylation as measured by western blot and quantified by the pSTAT5: total STAT5 ratio. As shown in FIGS. 6-9 and summarized in Table 3, all IL-2 polypeptides, except for 031, 032 and 033, activated pSTAT5 in HH cell line and only few had activity in NK-92 cells. These results confirmed that these IL-2 polypeptides did not bind IL-2Rα, but maintained wildtype IL-2's binding to IL-2Rβ and γ and the function to activate the same.

TABLE 3
Expression, stability, and bioactivity of CP trIL-2 Variants

| CP-trIL-2 | Expression (mg/L) | Stability (OD350) | Bioactivity |
|---|---|---|---|
| 011 | 79.22 | ++ | +++ |
| 012 | 86.91 | +++ | +++ |
| 013^{a} | 82.98 | +++ | +++ |
| 021 | 94.88 | ++ | +++ |
| 022 | 96.71 | ++ | +++ |
| 023 | 61.43 | + | ++ |
| 031 | 120.62 | + | − |
| 032 | 164.26 | +++ | − |
| 033 | 109.57 | +++ | − |
| 041 | 40.99 | + | + |
| 042 | 44.38 | +++ | ++ |
| 043 | 47.97 | +++ | ++ |
| 051 | 88.72 | +++ | ++ |
| 052 | 101.42 | + | + |
| 053^{a} | 76.04 | +++ | + |
| 061 | 67.11 | + | + |
| 062 | 110.21 | +++ | +++ |
| 063 | 54.69 | +++ | + |
| 071 | 168.96 | + | ++ |
| 072 | 140.18 | ++ | +++ |
| 073^{a} | 246.55 | +++ | +++ |
| 081 | Very Low | +++ | + |
| 082 | 59.63 | +++ | +++ |
| 083 | 76.52 | +++ | +++ |

^{a}precipitation during concentration

### 7.6.3 Example 3: CP-trIL-2 Activities on Human PBMC and Mouse Splenocytes

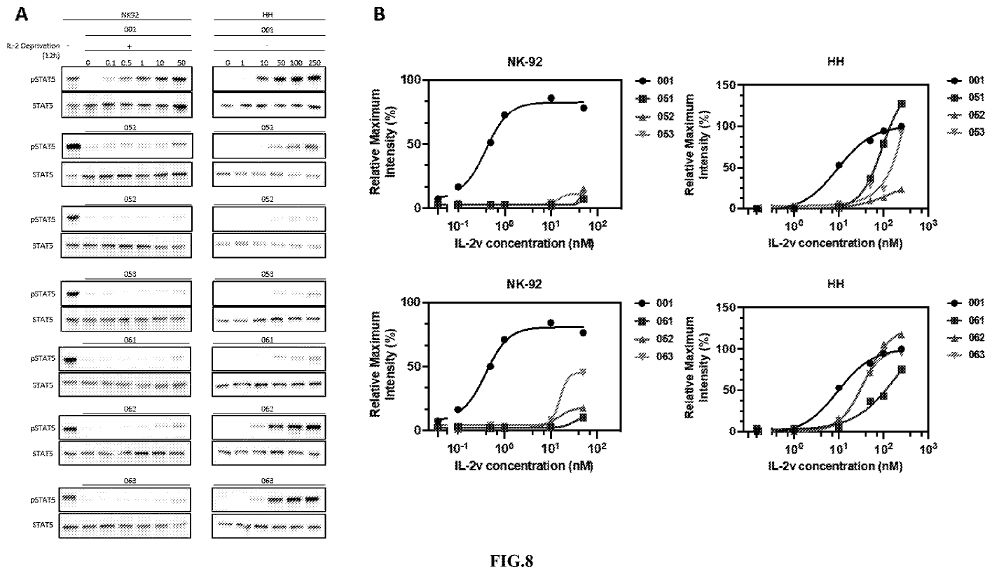

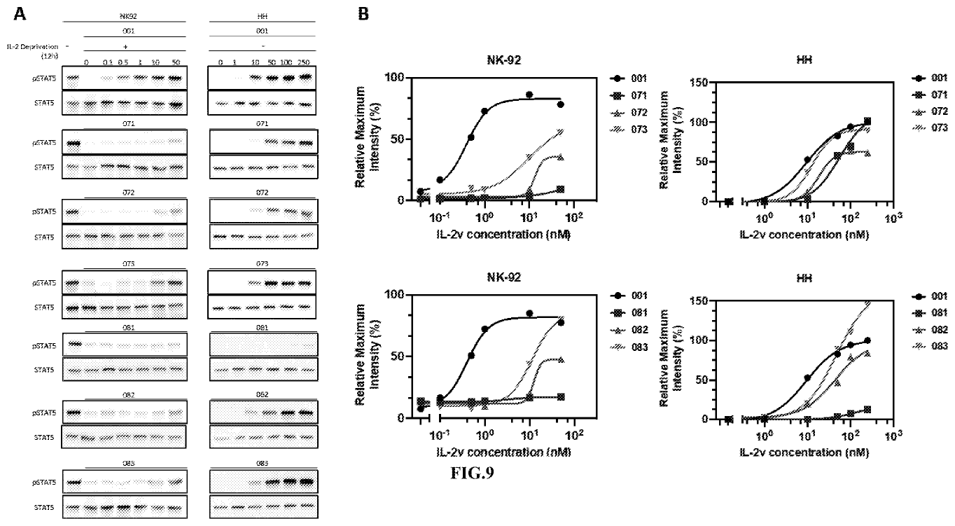

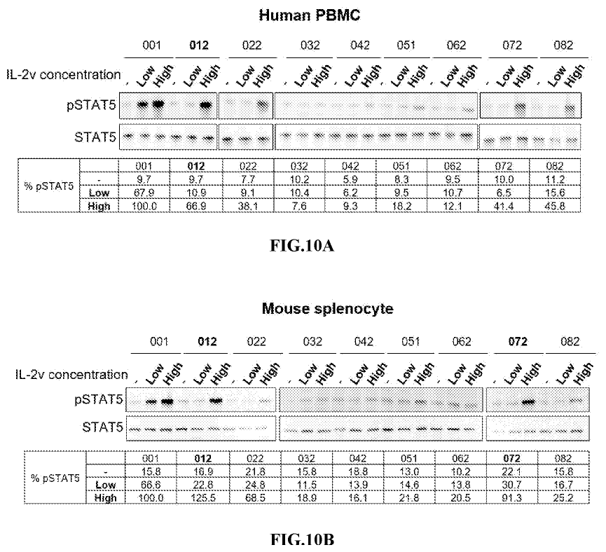

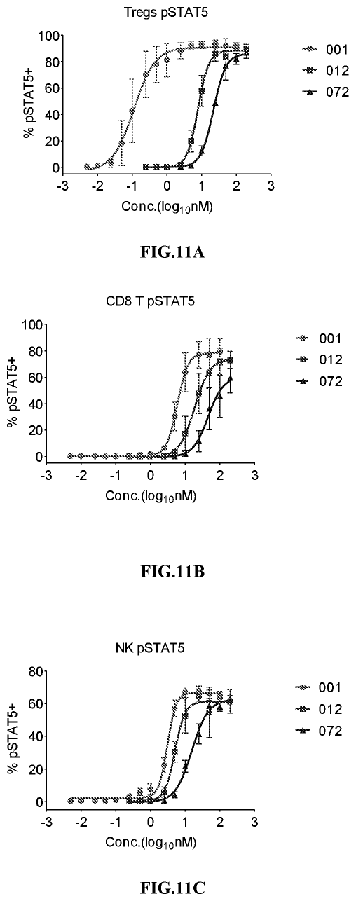

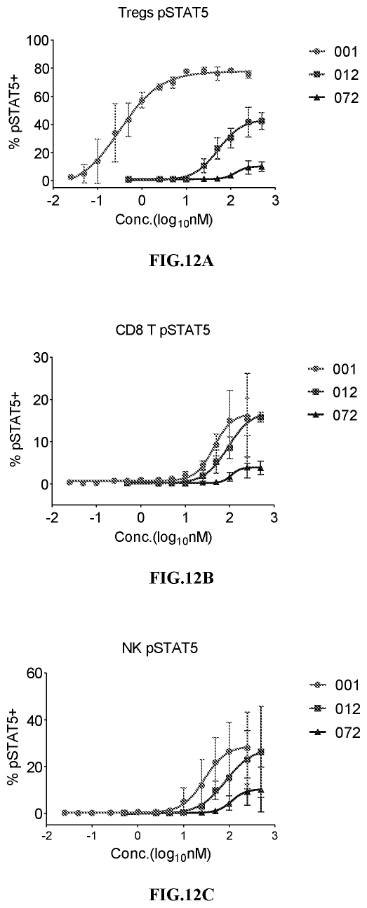

To confirm that contrary to its wildtype counterpart, the CP trIL-2 variants do not preferentially bind to the trimeric receptor IL-2Rαβγ over IL-2Rβγ, STAT5 phosphorylation assay was performed. Specifically, human PBMCs and mouse splenocytes were separately treated with the wild type hIL-2 (001) and the CP trIL-2 variants, respectively, and STAT5 phosphorylation was measured by western blot. The activation profiles of the IL-2 molecules were indicated by % pSTAT5, which was calculated by the relative intensity of pSTAT5 to STAT5, normalized with that of the sample treated wild type hIL-2 (001) at high concentration (25 nM). As shown in FIGS. 10A and 10B, treatment by wild type hIL-2 (001) at low concentration (2.5 nM) resulted in about 65% activation of pSTAT5 signaling in both healthy donor human PBMCs and naive Balb/c mouse splenocytes. By contrast, samples treated by CP trIL-2 variants at low concentration (2.5 nM) showed very little activation of pSTAT5 signaling (about 10% or less), indicating a loss of preference to high-affinity receptors, or reduced affinity to trimeric receptor IL-2Rαβγ. Nonetheless, comparable activation was observed in samples treated with certain CP trIL-2 variants at high concentration (25 nM) as compared to the wildtype treatment. As shown in FIG. 10A, treatment by variant 012 resulted in comparable activation as treatment by 001 in PBMC. As shown in FIG. 10B, treatment by variants 012 and 072 resulted in comparable activation as treatment by 001 in mouse splenocytes. These results confirmed that CP trIL-2 variants 012 and 072 retained their function to bind and activate the intermediate-affinity receptor IL-2Rβγ, but lost the preferential binding to the high-affinity receptor IL-2Rαβγ.

To determine the selective activation profiles of 012 and 072 in primary cells, pSTAT5 was measured in different cell populations of the healthy donor human PBMCs and the naive Balb/c mouse splenocytes, respectively. As shown in FIG. 11A, the potencies of 012, 072 and 001 to activate STAT5 signaling differed greatly in the CD4+ FOXP3+ Treg population of human PBMCs. 012 induced pSTAT5 in Tregs with an EC50 of 7.909 nM and 072 with an EC50 of 22.00 nM, while 001 (wildtype) induced pSTAT5 with an EC50 of 0.1122 nM (FIG. 11A; Table 4). By contrast, similar potencies were observed for 012, 072 and 001 (wildtype) treatment in non-activated CD8 T and NK cells, which expressed the intermediate-affinity IL-2R complex. Similarly, in mouse splenocytes, 012 and 072 showed significantly decreased potencies in Tregs, as compared to the wildtype (FIG. 11B; Table 4), but had similar EC50s to that of 001 in CD8+T and NK cells. As such, CP trIL-2 variants 012 and 072 were much less potent in inducing STAT5 activation in Tregs but were similarly potent in CD8+T and NK cells as compared to the wild type IL-2 (001).

TABLE 4
Potency of STAT5 activation by 012, 072 and 001 in
selected human and mouse lymphocyte populations

| | Human | | | Mouse | | |
|---|---|---|---|---|---|---|
| Lymphocyte | 001 EC₅₀ | 012 EC₅₀ | 072 EC₅₀ | 001 EC₅₀ | 012 EC₅₀ | 072 EC₅₀ |
| population | (nM) | (nM) | (nM) | (nM) | (nM) | (nM) |
| Tregs | 0.1122 | 7.909 | 22.00 | 0.3189 | 52.02 | 128.7 |
| CD8 T cells | 5.942 | 18.59 | 45.71 | 44.35 | 91.77 | 110.0 |
| NK cells | 3.098 | 5.11 | 15.64 | 28.57 | 84.02 | 113.4 |

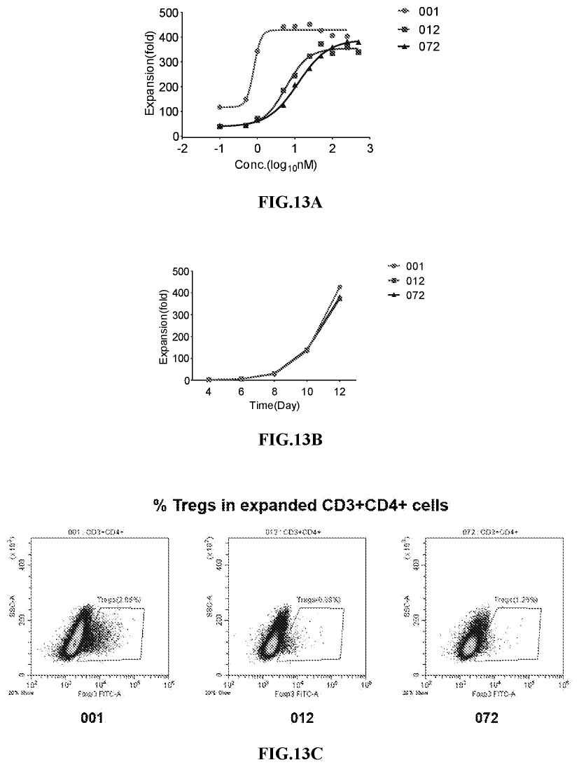

To confirm that the different activation profiles of the CP trIL-2 variants could translate to differences in human cellular responses, a cell proliferation assay was performed. PBMCs from healthy donors were cultured in the presence of CTS™ Dynabeads™ CD3/CD28 (Gibco 40203D) and either 012, 072 or 001 for 4 days, followed by 8 more days culture with the same IL-2 molecule (CP trIL-2 variant or wild type) but without anti-CD3/CD28 Dynabeads. As shown in FIG. 13A, 012 and 072 treatment expanded PBMCs to a similar extent as 001 treatment did, although higher concentrations of 012 and 072 were required for maximal proliferation. Expansion curves for PBMC treated with 50 nM 012, 072, and 001, respectively, demonstrated that the CP trIL-2 variants 012 and 072 were similarly effective as the wildtype in promoting the PBMC expansion at 50 nM (FIG. 13B). As shown in FIG. 13C, CP trIL-2 variants (012 and 072) were much less potent in promoting the expansion of Treg populations in vitro compared to the wildtype (001).

### 7.6.4 Example 4: In Vivo Toxicity and Activation Profiles

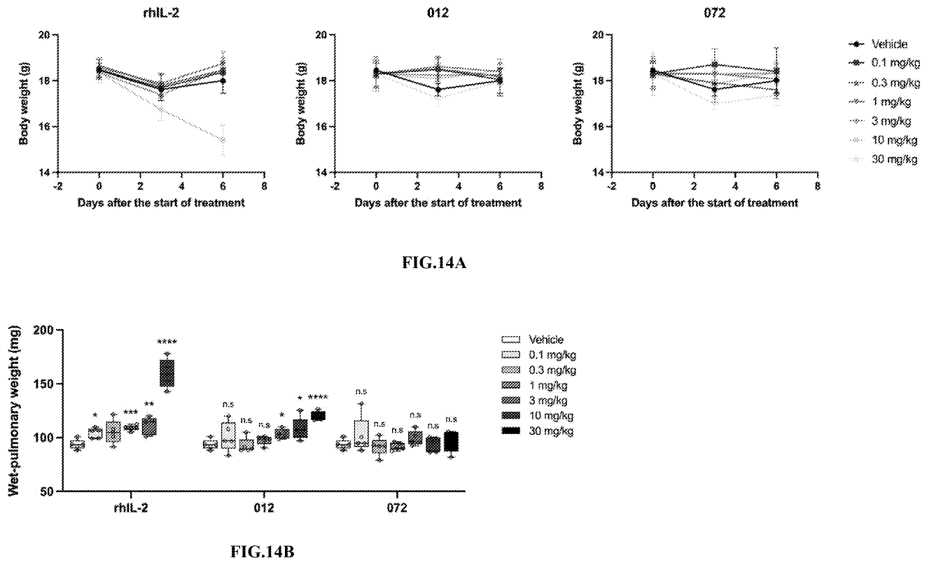

The toxicity of the CP trIL-2 variants was determined in comparison with the wildtype IL-2. Specifically, 18 groups of normal female Balb/c mice (n=6 per group) received IP injections of (1) vehicle (phosphate-buffered saline (PBS)), (2) 0.1 mg/kg, 0.3 mg/kg, 1 mg/kg, 3 mg/kg or 10 mg/kg rhIL-2 (Beijing Four Rings Bio-Pharmaceutical, Recombinant Human Interleukin-2 for Injection, Lot. S10970015) in PBS or (3) 0.1 mg/kg, 0.3 mg/kg, 1 mg/kg, 3 mg/kg, 10 mg/kg or 30 mg/kg 012 and 072 in PBS, respectively, once daily for 5 days. Mouse body weight was measured on Day 3 and Day 6. As shown in FIG. 14A, the mice injected with 10 mg/kg rhIL-2 experienced great weight loss, while those treated with 10 mg/kg or 30 mg/kg 012 or 072 did not show any significant body weight change. Additionally, pulmonary edema resulting from capillary leak at higher doses of rhIL-2 in mice was assessed by measuring the increase in wet pulmonary weight. On the morning of the 6th day, mice were euthanized. The lung tissue samples were dehydrated by incubation at 65° C. for 72 hours. The resulting dry lung weight was recorded and the weight from pulmonary fluid (wet lung weight) was calculated. As shown in FIG. 14B, the average wet pulmonary weight from the 30 mg/kg 012 or 072-treated group was significantly lower than that from the 10 mg/kg rhIL-2-treated group, and comparable to those from the lower dose groups, indicating that the severity of pulmonary edema was much lower in 012 and 072 treated groups compared with rhIL-2 treated groups. Taken together, these data demonstrate that 012 and 072, compared with rhIL-2, had significantly reduced toxicity in vivo.

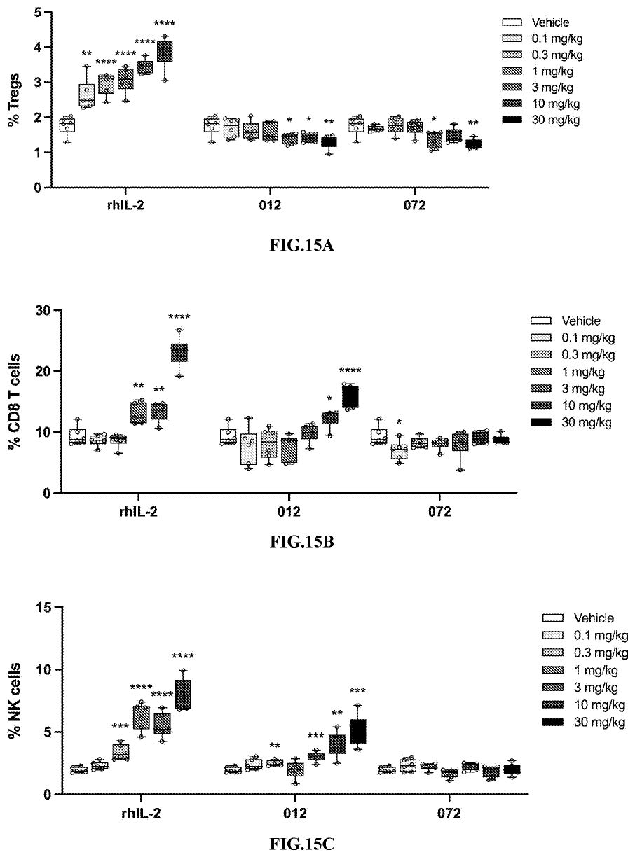

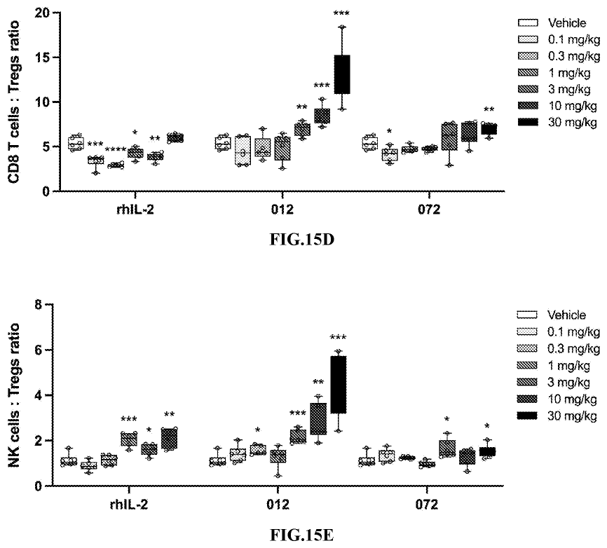

The selective activation profiles of the CP trIL-2 variants were also confirmed in vivo. The spleens of the mice treated as described above were harvested, and lymphocyte populations were analyzed by flow cytometry. Consistent with the in vitro findings, neither 012 nor 072 at any dose resulted in increased percentages of CD4+ Foxp3+ Tregs, while rhIL-2 resulted in a dose-dependent expansion of Tregs (FIG. 15A). Furthermore, both rhIL-2 and 012 showed dose-dependent activity in promoting the proliferation of both CD8 T and NK cell in vivo (FIGS. 15B-15C). As such, compared to rhIL-2, treatment with the CP trIL-2 variant 012 resulted in a significantly larger increase in the ratio of effector (CD8+T or NK cells) to regulatory (Tregs) T cells (FIGS. 15D-15E).

### 7.6.5 Example 5: In Vivo Anti-Tumor Efficacy

Efficacy studies were performed using female C57BL/6 mice (18-20 g) obtained from Charles River Laboratories (Beijing, China), n=6/group. For the MC38 model, female C57BL/6 mice (18-20 g) were implanted in the right flank with 5×10⁵ cells in 0.1 mL of phosphate-buffered saline (PBS). Treatment was initiated when the mean tumor size reached 80-100 mm³. Animals were administered vehicle, 012, or rhIL-2 by an intraperitoneal injection. rhIL-2 doses were 1 mg/kg and 3 mg/kg, while 012 doses were 10 mg/kg, 30 mg/kg, and 60 mg/kg, qdx5 then rest for 2 days and for another cycle.

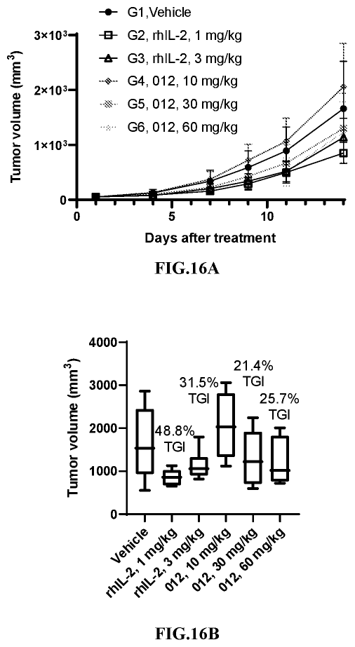

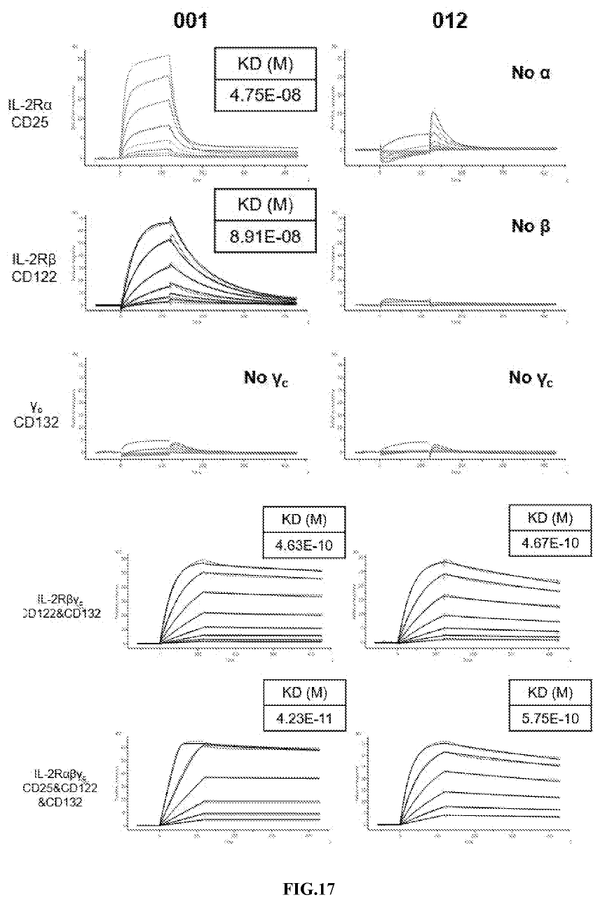

After tumor cell inoculation, the animals were checked daily for morbidity, mortality, effects of tumor growth, and effects of treatment on normal behaviors and appearance such as mobility, food and water consumption, eye/hair matting, and any other abnormal effects. Body weight gain/loss was measured three times weekly. Tumor volumes were measured three times weekly in two dimensions using a caliper, and the volume was expressed in mm³ using the formula: V=0.5 a×b² where a and b are the long and short diameters of the tumor, respectively. As a single agent in the mouse MC38 model, 30 mg/kg and 60 mg/kg 012 demonstrated tumor growth inhibition (TGI) over 20% (FIGS. 16A-16B).

All animal studies were conducted under accreditation by the Association for Assessment and Accreditation of Laboratory Animal Care (AAALAC). Animals were housed in individualized ventilated cages under controlled conditions of temperature, humidity, and light and fed a diet of sterilized dry granule food. All mice were adapted in the facility for at least 7 days. Euthanasia criteria consisted of either body weight drop of >20% for 72 hours, hypoactivity, hypothermia, respiratory distress, tumor volume reaching 3000 mm³ or termination of the study.

### 7.6.6 Example 6: Binding Affinities to IL-2Rs

The binding affinities of the CP trIL-2 variant 012 and the wildtype IL-2 (001) to various IL-2 receptors were determined by surface plasmon resonance (SPR). The human IL-2 receptors used in this study included IL-2Rα/CD25, IL-2Rβ/CD122, common γ receptor/CD132, IL-2Rβ&IL-2Rγ heterodimer protein, and IL-2Rβ&IL-2Rα&IL-2Rγ protein. The IL-2 receptor proteins were immobilized on a Biacore™ sensor chip CM5 according to manufacturer's protocol. 001 and 012 at different concentrations were passed over the surface of the chip in a stepwise model. Different concentrations of 001 or 012 were flowed over the bound ligand on the surface of the chip for 120 seconds, after which a blank solution was passed over the surface to allow the IL-2R to dissociate. The resulting sensorgrams were analyzed with the native instrument software to calculate the binding affinities. As shown in FIG. 17 and Table 5, the binding affinities of the CP trIL-2 variant 012 to single IL-2 receptors (IL-2Rα, IL-2Rβ, and common γ receptor) as well as IL-2Rαβγ were reduced compared to those of the wildtype IL-2, but it bound to the IL-2Rβγ receptor at an equally high affinity as the wildtype.

TABLE 5
Binding of 001 and 012 to IL-2Rα, IL-2Rβ, common γ receptor, IL-2Rβγ, and IL-2Rαβγ

| | Capture | | | | | | | |
|---|---|---|---|---|---|---|---|---|
| | level | | Analyte | Ka | Kd | | Rmax | Fit |
| Ligand | (RU) | Analyte | Conc. | (1/ms) | (1/s) | KD (M) | (RU) | model |
| Human IL- | 270.3 | 001 | 0.977- | NA | NA | 4.75E−08 | 36.5 | Steady |
| 2RA | | | 125 nM | | | | | state |
| | | | | | | | | affinity |
| Human IL- | 301.3 | 012 | 0.977- | No binding | | | | |
| 2RA | | | 250 nM | | | | | |
| Human IL- | 317.4 | 001 | 7.813- | 2.09E+05 | 1.86E−02 | 8.91E−08 | 97.8 | 1:1 |
| 2RB | | | 250 nM | | | | | binding |
| Human IL- | 289.2 | 012 | 7.813- | No binding | | | | |
| 2RB | | | 250 nM | | | | | |
| Human IL- | 36/9.4 | 001 | 0.977- | No binding | | | | |
| 2RG | | | 500 nM | | | | | |
| Human IL- | 301.5 | 012 | 0.977- | No binding | | | | |
| 2RG | | | 500 nM | | | | | |
| Human IL- | 523.2 | 001 | 0.488- | 6.70E+05 | 3.11E−04 | 4.63E−10 | 57.0 | 1:1 |
| 2RB&G | | | 62.5 nM | | | | | binding |
| Human IL- | 542.8 | 012 | 0.244- | 2.28E+06 | 1.06E−03 | 4.67E−10 | 29.8 | 1:1 |
| 2RB&G | | | 15.625 | | | | | binding |
| | | | nM | | | | | |
| Human IL- | 625.6 | 001 | 0.977- | 9.11E+06 | 3.86E−04 | 4.23E−11 | 63.0 | 1:1 |
| 2RB&IL- | | | 31.25 nM | | | | | binding |
| 2RA&IL- | | | | | | | | |
| 2RG | | | | | | | | |
| Human IL- | 685.2 | 012 | 0.977- | 1.27E+06 | 7.32E−04 | 5.75E−10 | 36.3 | 1:1 |
| 2RB&IL- | | | 31.25 nM | | | | | binding |
| 2RA&IL- | | | | | | | | |
| 2RG | | | | | | | | |

### 8. Exemplary Embodiments

Embodiment 1: An interleukin-2 (IL-2) polypeptide comprising, from N-terminus to C-terminus, (i) helix 1 (“H1”), helix 3 (“H3”), helix 2 (“H2”), and helix 4 (“H4”); or (ii) H2, H3, H1, and H4; or a circularly permutated variant thereof, wherein H1, H2, H3, and H4 each has the amino acid sequences of SEQ TD NOs:2, 5, 7, and 10, respectively, or a variant thereof having up to five amino acid mutations.

Embodiment 2: The IL-2 polypeptide of Embodiment 1 wherein the circularly permutated variant has a cutting point at the C-terminus of H1, H2, H3 or H4.

Embodiment 3: The IL-2 polypeptide of Embodiment 1 or 2, wherein: (1) H1 has the amino acid sequence of SEQ ID NO:2; (2) H2 has the amino acid sequence of SEQ ID NO: 5, 13, 14, or 15; (3) H3 has the amino acid sequence of SEQ ID NO: 7; or (4) H4 has the amino acid sequence of SEQ ID NO: 10, 16, 17, or 18; or any combination thereof.

Embodiment 4: The IL-2 polypeptide of any one of Embodiments 1 to 3, further comprising: (1) linker 1 (“L1”) located at the N-terminus of H1; (2) linker 2 (“L2”) located at the C-terminus of H1; (3) linker 3 (“L3”) located at the C-terminus of112; (4) linker 4 (“L4”) located at the C-terminus of H3; or (5) linker 5 (“L5”) located at the C-terminus of H4; or any combination thereof; wherein L1, L2, L3, L4, and L5 each has the amino acid sequences of SEQ ID NOs:1, 3, 6, 8, and 11, respectively, or a variant thereof having up to three amino acid mutations.

Embodiment 5: The IL-2 polypeptide of Embodiment 4, comprising (1) L1; (2) L2, except when H1 is located at the C-terminus of the IL-2 polypeptide; (3) L3, except when H2 is located at the C-terminus of the IL-2 polypeptide; (4) L4, except when H3 is located at the C-terminus of the IL-2 polypeptide; and (5) L5, except when H4 is located at the C-terminus of the IL-2 polypeptide.

Embodiment 6: The IL-2 polypeptide of Embodiment 4 or 5, wherein: (1) L1 has the amino acid sequence of SEQ ID NO:1 or 12; (2) L2 has the amino acid sequence of SEQ ID NO:3; (3) L3 has the amino acid sequence of SEQ ID NO:6; (4) L4 has the amino acid sequence of SEQ ID NO: 8; (5) L5 has the amino acid sequence of SEQ ID NO: 11; or any combination thereof.

Embodiment 7: The IL-2 polypeptide of any one of Embodiments 1 to 6, comprising from N-terminus to C-terminus, H1, H3, H2, and H4.

Embodiment 8: The IL-2 polypeptide of Embodiment 7 that has the amino acid sequence of SEQ ID NO:20, 21, or 22.

Embodiment 9: The IL-2 polypeptide of any one of Embodiments 1 to 6 comprising, from N-terminus to C-terminus, H3, H2, H4, and H1.

Embodiment 10: The IL-2 polypeptide of Embodiment 9 that has the amino acid sequence of SEQ ID NO:23, 24, or 25.

Embodiment 11: The IL-2 polypeptide of any one of Embodiments 1 to 6 comprising, from N-terminus to C-terminus, H2, H4, H1, and H3.

Embodiment 12: The IL-2 polypeptide of Embodiment 11 that has the amino acid sequence of SEQ ID NO:26, 27, or 28.

Embodiment 13: The IL-2 polypeptide of any one of Embodiments 1 to 6 comprising, from N-terminus to C-terminus, H4, H1, H3, and H2.

Embodiment 14: The IL-2 polypeptide of Embodiment 13 that has the amino acid sequence of SEQ ID NO:29, 30, or 31.

Embodiment 15: The IL-2 polypeptide of any one of Embodiments 1 to 6 comprising, from N-terminus to C-terminus, H1, H4, H2, and H3.

Embodiment 16: The IL-2 polypeptide of Embodiment 15 that has the amino acid sequence of SEQ ID NO:32, 33, or 34.

Embodiment 17: The IL-2 polypeptide of any one of Embodiments 1 to 6 comprising, from N-terminus to C-terminus, H4, H2, H3, and H1.

Embodiment 18: The IL-2 polypeptide of Embodiment 17 that has the amino acid sequence of SEQ ID NO:35, 36, or 37.

Embodiment 19: The IL-2 polypeptide of any one of Embodiments 1 to 6 comprising, from N-terminus to C-terminus, H2, H3, H1, and H4.

Embodiment 20: The IL-2 polypeptide of Embodiment 19 that has the amino acid sequence of SEQ ID NO:38, 39, or 40.

Embodiment 21: The IL-2 polypeptide of any one of Embodiments 1 to 6 comprising, from N-terminus to C-terminus, H3, H1, H4, and H2.

Embodiment 22: The IL-2 polypeptide of Embodiment 21 that has the amino acid sequence of SEQ ID NO:41, 42, or 43.

Embodiment 23: The IL-2 polypeptide of any one of Embodiments 1 to 22 that is conjugated to an Fc domain, a Human Serin Albumin (HSA), an anti-HSA binder, a polyethylene glycol (PEG), or a lipid moiety.

Embodiment 24: The IL-2 polypeptide of any one of Embodiments 1 to 23 that is linked to a targeting moiety that binds to a tumor-associate antigen or an antigen in the extracellular matrix in a tumor, an immunotherapeutic agent, an immune checkpoint modulator, or a peptide-MHC complex.

Embodiment 25: The IL-2 polypeptide of any one of Embodiments 1 to 24, wherein IL-2 polypeptide (1) has reduced or abolished binding affinity to IL-2Rα; (2) binds to IL-2Rβ/γ with an affinity equal to or greater than wildtype human IL-2's binding affinity for IL-2Rβ/γ; (3) activates IL-2Rβ/γ to an extent or amount equal to or great than wildtype human IL-2 activates IL-2Rβ/γ; or (4) has negligible immunogenicity in human; or any combination thereof.

Embodiment 26: A nucleic acid having a polynucleotide sequence encoding the IL-2 polypeptide of any one of Embodiments 1 to 25.

Embodiment 27: The nucleic acid of Embodiment 26 that is an mRNA.

Embodiment 28: A vector comprising the nucleic acid of Embodiment 26.

Embodiment 29: A host cell having the nucleic acid of Embodiment 26 or the vector of Embodiment 28.

Embodiment 30: A pharmaceutical composition comprising the IL-2 polypeptide of any one of Embodiments 1 to 25 and a pharmaceutically acceptable carrier.

Embodiment 31: A pharmaceutical composition comprising the nucleic acid of Embodiment 26 or 27 and a pharmaceutically acceptable carrier.

Embodiment 32: A kit comprising the IL-2 polypeptide of any one of Embodiments 1 to 25.

Embodiment 33: A kit comprising the nucleic acid of Embodiment 26 or 27.

Embodiment 34: An in vitro or ex vivo method of activating an immune effector cell, comprising contacting the immune effector cell with the IL-2 polypeptide of any one of Embodiments 1 to 25 under conditions to activate the immune effector cell.

Embodiment 35: The method of Embodiment 34 wherein the immune effector cell is a T cell, a NK cell, a NKT cell, or a myeloid cell.

Embodiment 36: The method of Embodiment 34 wherein the immune effector cell is a T cell selected from a CD4+ T cell, a CD8+ T cell, a Th cell, a Tc cell, and a CAR T cell.

Embodiment 37: A method of enhancing an immune response in a subject in need thereof, comprising administering a therapeutically effective amount of the IL-2 polypeptide of any one of Embodiments 1 to 25, the nucleic acid of Embodiment 26 or 27, or the pharmaceutical composition of Embodiment 30 or 31 to the subject.

Embodiment 38: Use of the IL-2 polypeptide of any one of Embodiments 1 to 25, the nucleic acid of Embodiment 26 or 27, or the pharmaceutical composition of Embodiment 30 or 31 in enhancing an immune response in a subject in need thereof.

Embodiment 39: The method of Embodiment 37 or use of Embodiment 38, wherein the subject has cancer.

Embodiment 40: A method of treating cancer in a subject in need thereof comprising administering a therapeutically effective amount of the IL-2 polypeptide of any one of Embodiments 1 to 25, the nucleic acid of Embodiment 26 or 27, or the pharmaceutical composition of Embodiment 30 or 31 to the subject.

Embodiment 41: Use of the IL-2 polypeptide of any one of Embodiments 1 to 25, the nucleic acid of Embodiment 26 or 27, or the pharmaceutical composition of Embodiment 30 or 31 in treating cancer in a subject in need thereof.

Embodiment 42: The method or use of any one of Embodiments 39 to 41, wherein the cancer is a hematological cancer or a solid tumor.

Embodiment 43: The method or use of any one of Embodiments 37 to 42, wherein the IL-2 polypeptide, the nucleic acid, or the pharmaceutical composition is administered intratumorally, intravenously, subcutaneously, intraosseously, orally, transdermally, or sublingually.

Embodiment 44: The method or use of any one of Embodiments 37 to 43, wherein the subject exhibits reduced adverse events as compared to a subject treated with human wildtype IL-2.

Embodiment 45: The method or use of any one of Embodiments 37 to 44, wherein the IL-2 polypeptide, the nucleic acid, or the pharmaceutical composition is administered in combination with a second therapeutic agent.

Embodiment 46: The method or use of Embodiment 45, wherein the second therapeutic agent is a chemotherapeutic agent, a hormonal agent, an antitumor agent, an immunostimulatory agent, an immunomodulator, an immunotherapeutic agent or any combination thereof.

What is claimed is:

1. An interleukin-2 (IL-2) polypeptide comprising, from N-terminus to C-terminus, (i) helix 1 (“H1”), helix 3 (“H3”), helix 2 (“H2”), and helix 4 (“H4”); or (ii) H2, H3, H1, and H4; or a circularly permutated variant thereof,
wherein H1, H2, H3, and H4 each has the amino acid sequences of SEQ ID NOs:2, 5, 7, and 10, respectively, or a variant thereof having up to five amino acid mutations.

2. The IL-2 polypeptide of claim 1 wherein the circularly permutated variant has a cutting point at the C-terminus of H1, H2, H3 or H4.

3. The IL-2 polypeptide of claim 1 or 2, wherein:
(1) H1 has the amino acid sequence of SEQ ID NO:2;
(2) H2 has the amino acid sequence of SEQ ID NO:5, 13, 14, or 15;
(3) H3 has the amino acid sequence of SEQ ID NO:7; or
(4) H4 has the amino acid sequence of SEQ ID NO:10, 16, 17, or 18; or any combination thereof.

4. The IL-2 polypeptide of any one of claims 1 to 3, further comprising:
(1) linker 1 (“L1”) located at the N-terminus of H1;
(2) linker 2 (“L2”) located at the C-terminus of H1;
(3) linker 3 (“L3”) located at the C-terminus of H2;
(4) linker 4 (“L4”) located at the C-terminus of H3; or
(5) linker 5 (“L5”) located at the C-terminus of H4; or any combination thereof;
wherein L1, L2, L3, L4, and L5 each has the amino acid sequences of SEQ ID NOs:1, 3, 6, 8, and 11, respectively, or a variant thereof having up to three amino acid mutations.

5. The IL-2 polypeptide of claim 4, comprising
(1) L1;
(2) L2, except when H1 is located at the C-terminus of the IL-2 polypeptide;
(3) L3, except when H2 is located at the C-terminus of the IL-2 polypeptide;
(4) L4, except when H3 is located at the C-terminus of the IL-2 polypeptide; and
(5) L5, except when H4 is located at the C-terminus of the IL-2 polypeptide.

6. The IL-2 polypeptide of claim 4 or 5, wherein:
(1) L1 has the amino acid sequence of SEQ ID NO:1 or 12;
(2) L2 has the amino acid sequence of SEQ ID NO:3;
(3) L3 has the amino acid sequence of SEQ ID NO:6;
(4) L4 has the amino acid sequence of SEQ ID NO:8;
(5) L5 has the amino acid sequence of SEQ ID NO:11; or any combination thereof.

7. The IL-2 polypeptide of any one of claims 1 to 6, comprising from N-terminus to C-terminus, H1, H3, H2, and H4.

8. The IL-2 polypeptide of claim 7 that has the amino acid sequence of SEQ ID NO:20, 21, or 22.

9. The IL-2 polypeptide of any one of claims 1 to 6 comprising, from N-terminus to C-terminus, H3, H2, H4, and H1.

10. The IL-2 polypeptide of claim 9 that has the amino acid sequence of SEQ ID NO:23, 24, or 25.

11. The IL-2 polypeptide of any one of claims 1 to 6 comprising, from N-terminus to C-terminus, H2, H4, H1, and H3.

12. The IL-2 polypeptide of claim 11 that has the amino acid sequence of SEQ ID NO:26, 27, or 28.

13. The IL-2 polypeptide of any one of claims 1 to 6 comprising, from N-terminus to C-terminus, H4, H1, H3, and H2.

14. The IL-2 polypeptide of claim 13 that has the amino acid sequence of SEQ ID NO:29, 30, or 31.

15. The IL-2 polypeptide of any one of claims 1 to 6 comprising, from N-terminus to C-terminus, H1, H4, H2, and H3.

16. The IL-2 polypeptide of claim 15 that has the amino acid sequence of SEQ ID NO:32, 33, or 34.

17. The IL-2 polypeptide of any one of claims 1 to 6 comprising, from N-terminus to C-terminus, H4, H2, H3, and H1.

18. The IL-2 polypeptide of claim 17 that has the amino acid sequence of SEQ ID NO:35, 36, or 37.

19. The IL-2 polypeptide of any one of claims 1 to 6 comprising, from N-terminus to C-terminus, H2, H3, H1, and H4.

20. The IL-2 polypeptide of claim 19 that has the amino acid sequence of SEQ ID NO:38, 39, or 40.

21. The IL-2 polypeptide of any one of claims 1 to 6 comprising, from N-terminus to C-terminus, H3, H1, H4, and H2.

22. The IL-2 polypeptide of claim 21 that has the amino acid sequence of SEQ ID NO:41, 42, or 43.

23. The IL-2 polypeptide of any one of claims 1 to 22 that is conjugated to an Fc domain, a Human Serin Albumin (HSA), an anti-HSA binder, a polyethylene glycol (PEG), or a lipid moiety.

24. The IL-2 polypeptide of any one of claims 1 to 23 that is linked to a targeting moiety that binds to a tumor-associate antigen or an antigen in the extracellular matrix in a tumor, an immunotherapeutic agent, an immune checkpoint modulator, or a peptide-MHC complex.

25. The IL-2 polypeptide of any one of claims 1 to 24, wherein IL-2 polypeptide
(1) has reduced or abolished binding affinity to IL-2Rα;
(2) binds to IL-2Rβ/γ with an affinity equal to or greater than wildtype human IL-2's binding affinity for IL-2Rβ/γ;
(3) activates IL-2Rβ/γ to an extent or amount equal to or great than wildtype human IL-2 activates IL-2Rβ/γ; or
(4) has negligible immunogenicity in human; or any combination thereof.

26. A nucleic acid having a polynucleotide sequence encoding the IL-2 polypeptide of any one of claims 1 to 25.

27. The nucleic acid of claim 26 that is an mRNA.

28. A vector comprising the nucleic acid of claim 26.

29. A host cell having the nucleic acid of claim 26 or the vector of claim 28.

30. A pharmaceutical composition comprising the IL-2 polypeptide of any one of claims 1 to 25 and a pharmaceutically acceptable carrier.

31. A pharmaceutical composition comprising the nucleic acid of claim 26 or 27 and a pharmaceutically acceptable carrier.

32. A kit comprising the IL-2 polypeptide of any one of claims 1 to 25.

33. A kit comprising the nucleic acid of claim 26 or 27.

34. An in vitro or ex vivo method of activating an immune effector cell, comprising contacting the immune effector cell with the IL-2 polypeptide of any one of claims 1 to 25 under conditions to activate the immune effector cell.

35. The method of claim 34 wherein the immune effector cell is a T cell, a NK cell, a NKT cell, or a myeloid cell.

36. The method of claim 34 wherein the immune effector cell is a T cell selected from a CD4+ T cell, a CD8+ T cell, a Th cell, a Tc cell, and a CAR T cell.

37. A method of enhancing an immune response in a subject in need thereof, comprising administering a therapeutically effective amount of the IL-2 polypeptide of any one of claims 1 to 25, the nucleic acid of claim 26 or 27, or the pharmaceutical composition of claim 30 or 31 to the subject.

38. Use of the IL-2 polypeptide of any one of claims 1 to 25, the nucleic acid of claim 26 or 27, or the pharmaceutical composition of claim 30 or 31 in enhancing an immune response in a subject in need thereof.

39. The method of claim 37 or use of claim 38, wherein the subject has cancer.

40. A method of treating cancer in a subject in need thereof comprising administering a therapeutically effective amount of the IL-2 polypeptide of any one of claims 1 to 25, the nucleic acid of claim 26 or 27, or the pharmaceutical composition of claim 30 or 31 to the subject.

41. Use of the IL-2 polypeptide of any one of claims 1 to 25, the nucleic acid of claim 26 or 27, or the pharmaceutical composition of claim 30 or 31 in treating cancer in a subject in need thereof.

42. The method or use of any one of claims 39 to 41, wherein the cancer is a hematological cancer or a solid tumor.

43. The method or use of any one of claims 37 to 42, wherein the TL-2 polypeptide, the nucleic acid, or the pharmaceutical composition is administered intratumorally, intravenously, subcutaneously, intraosseously, orally, transdermally, or sublingually.

44. The method or use of any one of claims 37 to 43, wherein the subject exhibits reduced adverse events as compared to a subject treated with human wildtype IL-2.

45. The method or use of any one of claims 37 to 44, wherein the TL-2 polypeptide, the nucleic acid, or the pharmaceutical composition is administered in combination with a second therapeutic agent.

46. The method or use of claim 45, wherein the second therapeutic agent is a chemotherapeutic agent, a hormonal agent, an antitumor agent, an immunostimulatory agent, an immunomodulator, an immunotherapeutic agent or any combination thereof.


SEQUENCE LISTING (46 sequences)

SEQ ID NO: 1 - protein, 5 aa, synthetic construct
  REGION 1..5: note = Linker 1
APTSS

SEQ ID NO: 2 - protein, 24 aa, synthetic construct
  REGION 1..24: note = Helix 1
STKKTQLQLEHLLLDLQMILNGIN

SEQ ID NO: 3 - protein, 5 aa, synthetic construct
  REGION 1..5: note = Linker 2
NYKNP

SEQ ID NO: 4 - protein, 17 aa, synthetic construct
  REGION 1..17: note = Loop 1
KLTRMLTFKFYMPKKAT

SEQ ID NO: 5 - protein, 20 aa, synthetic construct
  REGION 1..20: note = Helix 2
ELKHLQCLEEELKPLEEVLN

SEQ ID NO: 6 - protein, 8 aa, synthetic construct
  REGION 1..8: note = Linker 3
LAQSKNFH

SEQ ID NO: 7 - protein, 19 aa, synthetic construct
  REGION 1..19: note = Helix 3
LRPRDLISNINVIVLELKG

SEQ ID NO: 8 - protein, 4 aa, synthetic construct
  REGION 1..4: note = Linker 4
SETT

SEQ ID NO: 9 - protein, 10 aa, synthetic construct
  REGION 1..10: note = Loop 2
FMCEYADETA

SEQ ID NO: 10 - protein, 21 aa, synthetic construct
  REGION 1..21: note = Helix 4
TIVEFLNRWITFCQSIISTLT

SEQ ID NO: 11 - protein, 10 aa, synthetic construct
  REGION 1..10: note = Linker 5
GGGGSGGGGS

SEQ ID NO: 12 - protein, 4 aa, synthetic construct
  REGION 1..4: note = Linker 1-v1
PTSS

SEQ ID NO: 13 - protein, 20 aa, synthetic construct
  REGION 1..20: note = Helix 2-v1 (C58S)
ELKHLQSLEEELKPLEEVLN

SEQ ID NO: 14 - protein, 20 aa, synthetic construct
  REGION 1..20: note = Helix 2-v2 (P65D)
ELKHLQCLEEELKDLEEVLN

SEQ ID NO: 15 - protein, 20 aa, synthetic construct
  REGION 1..20: note = Helix 2-v3 (C58S; P65D)
ELKHLQSLEEELKDLEEVLN

SEQ ID NO: 16 - protein, 21 aa, synthetic construct
  REGION 1..21: note = Helix 4-v1 (F117K)
TIVEKLNRWITFCQSIISTLT

SEQ ID NO: 17 - protein, 21 aa, synthetic construct
  REGION 1..21: note = Helix 4-v2 (F124K)
TIVEFLNRWITKCQSIISTLT

SEQ ID NO: 18 - protein, 21 aa, synthetic construct
  REGION 1..21: note = Helix 4-v3 (F117K and F124K)
TIVEKLNRWITKCQSIISTLT

SEQ ID NO: 19 - protein, 133 aa, Homo sapiens [sequence omitted]

SEQ ID NO: 20 - protein, 105 aa, synthetic construct
  REGION 1..105: note = 011
PTSSSTKKTQLQLEHLLLDLQMILNGINNYKNPLRPRDLISNINVIVLELKGSETTELKHLQSLEEELKPLEEVLNLAQSKNFHTIVEFLNRWITFSQSIISTLT

SEQ ID NO: 21 - protein, 105 aa, synthetic construct
  REGION 1..105: note = 012
PTSSSTKKTQLQLEHLLLDLQMILNGINNYKNPLRPRDLISNINVIVLELKGSETTELKHLQSLEEELKDLEEVLNLAQSKNFHTIVEFLNRWITFSQSIISTLT

SEQ ID NO: 22 - protein, 105 aa, synthetic construct
  REGION 1..105: note = 013
PTSSSTKKTQLQLEHLLLDLQMILNGINNYKNPLRPRDLISNINVIVLELKGSETTELKHLQSLEEELKDLEEVLNLAQSKNFHTIVEKLNRWITKSQSIISTLT

SEQ ID NO: 23 - protein, 110 aa, synthetic construct
  REGION 1..110: note = 021
LRPRDLISNINVIVLELKGSETTELKHLQSLEEELKPLEEVLNLAQSKNFHTIVEFLNRWITFSQSIISTLTGGGGSGGGGSPTSSSTKKTQLQLEHLLLDLQMILNGIN

SEQ ID NO: 24 - protein, 110 aa, synthetic construct
  REGION 1..110: note = 022
LRPRDLISNINVIVLELKGSETTELKHLQSLEEELKDLEEVLNLAQSKNFHTIVEFLNRWITFSQSIISTLTGGGGSGGGGSPTSSSTKKTQLQLEHLLLDLQMILNGIN

SEQ ID NO: 25 - protein, 110 aa, synthetic construct
  REGION 1..110: note = 023
LRPRDLISNINVIVLELKGSETTELKHLQSLEEELKDLEEVLNLAQSKNFHTIVEKLNRWITKSQSIISTLTGGGGSGGGGSPTSSSTKKTQLQLEHLLLDLQMILNGIN

SEQ ID NO: 26 - protein, 111 aa, synthetic construct
  REGION 1..111: note = 031
ELKHLQSLEEELKPLEEVLNLAQSKNFHTIVEFLNRWITFSQSIISTLTGGGGSGGGGSPTSSSTKKTQLQLEHLLLDLQMILNGINNYKNPLRPRDLISNINVIVLELKG

SEQ ID NO: 27 - protein, 111 aa, synthetic construct
  REGION 1..111: note = 032
ELKHLQSLEEELKDLEEVLNLAQSKNFHTIVEFLNRWITFSQSIISTLTGGGGSGGGGSPTSSSTKKTQLQLEHLLLDLQMILNGINNYKNPLRPRDLISNINVIVLELKG

SEQ ID NO: 28 - protein, 111 aa, synthetic construct
  REGION 1..111: note = 033
ELKHLQSLEEELKDLEEVLNLAQSKNFHTIVEKLNRWITKSQSIISTLTGGGGSGGGGSPTSSSTKKTQLQLEHLLLDLQMILNGINNYKNPLRPRDLISNINVIVLELKG

SEQ ID NO: 29 - protein, 107 aa, synthetic construct
  REGION 1..107: note = 041
TIVEFLNRWITFSQSIISTLTGGGGSGGGGSPTSSSTKKTQLQLEHLLLDLQMILNGINNYKNPLRPRDLISNINVIVLELKGSETTELKHLQSLEEELKPLEEVLN

SEQ ID NO: 30 - protein, 107 aa, synthetic construct
  REGION 1..107: note = 042
TIVEFLNRWITFSQSIISTLTGGGGSGGGGSPTSSSTKKTQLQLEHLLLDLQMILNGINNYKNPLRPRDLISNINVIVLELKGSETTELKHLQSLEEELKDLEEVLN

SEQ ID NO: 31 - protein, 107 aa, synthetic construct
  REGION 1..107: note = 043
TIVEKLNRWITKSQSIISTLTGGGGSGGGGSPTSSSTKKTQLQLEHLLLDLQMILNGINNYKNPLRPRDLISNINVIVLELKGSETTELKHLQSLEEELKDLEEVLN

SEQ ID NO: 32 - protein, 111 aa, synthetic construct
  REGION 1..111: note = 051
PTSSSTKKTQLQLEHLLLDLQMILNGINNYKNPTIVEFLNRWITFSQSIISTLTGGGGSGGGGSELKHLQSLEEELKPLEEVLNLAQSKNFHLRPRDLISNINVIVLELKG

SEQ ID NO: 33 - protein, 111 aa, synthetic construct
  REGION 1..111: note = 052
PTSSSTKKTQLQLEHLLLDLQMILNGINNYKNPTIVEFLNRWITFSQSIISTLTGGGGSGGGGSELKHLQSLEEELKDLEEVLNLAQSKNFHLRPRDLISNINVIVLELKG

SEQ ID NO: 34 - protein, 111 aa, synthetic construct
  REGION 1..111: note = 053
PTSSSTKKTQLQLEHLLLDLQMILNGINNYKNPTIVEKLNRWITKSQSIISTLTGGGGSGGGGSELKHLQSLEEELKDLEEVLNLAQSKNFHLRPRDLISNINVIVLELKG

SEQ ID NO: 35 - protein, 110 aa, synthetic construct
  REGION 1..110: note = 061
TIVEFLNRWITFSQSIISTLTGGGGSGGGGSELKHLQSLEEELKPLEEVLNLAQSKNFHLRPRDLISNINVIVLELKGSETTPTSSSTKKTQLQLEHLLLDLQMILNGIN

SEQ ID NO: 36 - protein, 110 aa, synthetic construct
  REGION 1..110: note = 062
TIVEFLNRWITFSQSIISTLTGGGGSGGGGSELKHLQSLEEELKDLEEVLNLAQSKNFHLRPRDLISNINVIVLELKGSETTPTSSSTKKTQLQLEHLLLDLQMILNGIN

SEQ ID NO: 37 - protein, 110 aa, synthetic construct
  REGION 1..110: note = 063
TIVEKLNRWITKSQSIISTLTGGGGSGGGGSELKHLQSLEEELKDLEEVLNLAQSKNFHLRPRDLISNINVIVLELKGSETTPTSSSTKKTQLQLEHLLLDLQMILNGIN

SEQ ID NO: 38 - protein, 105 aa, synthetic construct
  REGION 1..105: note = 071
ELKHLQSLEEELKPLEEVLNLAQSKNFHLRPRDLISNINVIVLELKGSETTPTSSSTKKTQLQLEHLLLDLQMILNGINNYKNPTIVEFLNRWITFSQSIISTLT

SEQ ID NO: 39 - protein, 105 aa, synthetic construct
  REGION 1..105: note = 072
ELKHLQSLEEELKDLEEVLNLAQSKNFHLRPRDLISNINVIVLELKGSETTPTSSSTKKTQLQLEHLLLDLQMILNGINNYKNPTIVEFLNRWITFSQSIISTLT

SEQ ID NO: 40 - protein, 105 aa, synthetic construct
  REGION 1..105: note = 073
ELKHLQSLEEELKDLEEVLNLAQSKNFHLRPRDLISNINVIVLELKGSETTPTSSSTKKTQLQLEHLLLDLQMILNGINNYKNPTIVEKLNRWITKSQSIISTLT

SEQ ID NO: 41 - protein, 107 aa, synthetic construct
  REGION 1..107: note = 081
LRPRDLISNINVIVLELKGSETTPTSSSTKKTQLQLEHLLLDLQMILNGINNYKNPTIVEFLNRWITFSQSIISTLTGGGGSGGGGSELKHLQSLEEELKPLEEVLN

SEQ ID NO: 42 - protein, 107 aa, synthetic construct
  REGION 1..107: note = 082
LRPRDLISNINVIVLELKGSETTPTSSSTKKTQLQLEHLLLDLQMILNGINNYKNPTIVEFLNRWITFSQSIISTLTGGGGSGGGGSELKHLQSLEEELKDLEEVLN

SEQ ID NO: 43 - protein, 107 aa, synthetic construct
  REGION 1..107: note = 083
LRPRDLISNINVIVLELKGSETTPTSSSTKKTQLQLEHLLLDLQMILNGINNYKNPTIVEKLNRWITKSQSIISTLTGGGGSGGGGSELKHLQSLEEELKDLEEVLN

SEQ ID NO: 44 - protein, 5 aa, synthetic construct
  REGION 1..5: note = linker
  VARIANT 1..5: note = (GGGGS)n, n=1, 2, 3, 4, or 5
GGGGS

SEQ ID NO: 45 - protein, 5 aa, synthetic construct
  REGION 1..5: note = linker
  VARIANT 1..5: note = (EAAAK)n, n=1, 2, 3, 4, or 5
EAAAK

SEQ ID NO: 46 - protein, 5 aa, synthetic construct
  REGION 1..3: note = linker
  VARIANT 1..2: note = (PA)nPAP, n=1, 2, 3, or 4
PAPAP